(19) United States
(12) Patent Application Publication
(10) Pub. No.: US 2022/0275027 A1
(43) Pub. Date: Sep. 1, 2022

(54) ATYPICAL SPLIT INTEINS AND USES THEREOF

(71) Applicants: TRUSTEES OF PRINCETON UNIVERSITY, Princeton, NJ (US); ALBERT EINSTEIN COLLEGE OF MEDICINE, Bronx, NY (US)

(72) Inventors: TOM W. MUIR, Princeton, NJ (US); ADAM STEVENS, Princeton, NJ (US); JOSEF GRAMESPACHER, Princeton, NJ (US); DAVID COWBURN, Bronx, NY (US); GIRIDHAR SEKAR, Bronx, NY (US)

(21) Appl. No.: 17/753,299

(22) PCT Filed: Aug. 28, 2019
(86) PCT No.: PCT/US2019/048508

§ 371 Date: Feb. 25, 2022

(51) Int. Cl. C07K 14/00; C07K 1/02

(52) U.S. Cl. CPC C07K 14/001; C07K 1/026; C07K 2319/00

(57) ABSTRACT

The present disclosure relates to atypical split N- and C-inteins and variants thereof. This disclosure also relates to complexes comprising the split N- or C-inteins of this disclosure and a compound of interest and compositions comprising said complexes. In addition, this disclosure relates to methods of using the atypical split N- and C-inteins.

### GOVERNMENT LICENSE RIGHTS

This invention was made with government support under Grant Nos. GM086868, OD016305, RR015495 and OD016432 awarded by the National Institutes of Health. The government has certain rights in the invention.

### FIELD OF THE DISCLOSURE

The present disclosure is comprised within the field of biotechnology, it specifically relates to split inteins and their uses.

### BACKGROUND

An intein is an intervening protein domain that undergoes a posttranslational auto-processing event called protein splicing in which it excises itself from a host protein while tracelessly ligating its flanking polypeptide sequences (exteins) to form a native peptide bond. Most inteins are found as contiguous domains embedded within a single gene and splice in cis. However, some exist naturally in split form, whereby each intein fragment is encoded on a separately expressed gene and must first associate prior to splicing in trans. These split inteins are commonly applied as tools in protein engineering, and are especially amenable to use in the cellular environment due to their highly specific recognition and unique activity.

Despite the growing use of inteins in chemical biology, their practical utility has been constrained by a number of common characteristics, namely (i) slow kinetics, (ii) context dependent efficiency with respect to the immediate flanking extein sequences, (iii) low expression levels of recombinant fusions to other proteins and (iv) suboptimal stability.

Thus, a need exists for more robust and more efficient split inteins for use in a variety of protein purification and protein modification applications.

### SUMMARY

The authors of this disclosure provide herewith split inteins with atypical split sites which exhibit accelerated splicing rates and activity under adverse conditions, as it is shown in example 1 (FIG. 5, tables 5 and 6) of the present application. The disclosed inteins are useful in the N-terminal modification of expressed proteins and would complement other reported methods for protein N-terminal modification, such as expressed protein ligation, transpeptidase-based ligation strategies, and various protein chemistry methods. In this regard, as the N-terminal intein fragments of these inteins are strikingly short, the isolated polypeptides are ideally suited for use in a range of protein modifications, since the complex protein of interest-split intein N-fragment can be easily obtained using solid-phase peptide synthesis.

Thus, an aspect of this disclosure relates to a split intein N-fragment comprising the amino acid sequence of SEQ ID NO: 1 or a variant thereof having at least 90% sequence identity with SEQ ID NO: 1.

Another aspect of this disclosure relates to a complex comprising:

(i) a compound of interest,
(ii) the split intein N-fragment of this disclosure, or a split intein N-fragment comprising the amino acid sequence selected from the group consisting of SEQ ID NO: 103-110,
wherein the complex optionally comprises a linker between (i) and (ii) and
wherein
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.

Another aspect of this disclosure relates to a split intein C-fragment comprising the amino acid sequence of SEQ ID NO: 7 or a variant thereof having at least 88% sequence identity with SEQ ID NO: 7.

Another aspect of this disclosure relates to a complex comprising:

(i) the split intein C-fragment of this disclosure or a split intein C-fragment comprising a sequence selected from the group consisting of SEQ ID NO: 114-120 and
(ii) a compound of interest
wherein the complex optionally comprises a linker between (i) and (ii) and
wherein
the compound of interest is bound to the C-terminus of the split intein C-fragment by an amide linkage or
if the complex comprises a linker, the compound of interest is bound to the linker by an amide linkage and/or the linker is bound to the C-terminus of the split intein C-fragment by an amide linkage.

In another aspect, this disclosure relates to a composition comprising the first complex and the second complex of this disclosure.

Another aspect of this disclosure relates to a complex comprising:
(i) the split intein C-fragment of this disclosure or a split intein C-fragment comprising a sequence selected from the group consisting of SEQ ID NO: 114-120
(ii) a compound of interest and
(iii) the split intein N-fragment of this disclosure, or a split intein N-fragment comprising the amino acid sequence selected from the group consisting of SEQ ID NO: 103-110
   wherein the complex optionally comprises a linker between (i) and (ii) and/or between (ii) and (iii),
   wherein
the compound of interest is linked to the C-terminus of the split intein C-fragment by an amide linkage or
if the complex comprises a linker, the compound of interest is bound to the linker by an amide linkage and/or the linker is bound to the C-terminus of the split intein C-fragment by an amide linkage and
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.

Another aspect of this disclosure relates to a conjugate comprising (a) the first complex of this disclosure and (b) a split intein C-fragment comprising the amino acid sequence of SEQ ID NO: 7 or a variant thereof having at least 88% sequence identity with SEQ ID NO: 7 or an amino acid sequence selected from the group consisting of SEQ ID NO: 114-120, wherein the C-terminus of the split intein N-fragment is linked to the N-terminus of the split intein C-fragment by a peptide bond.

In another aspect, this disclosure relates to a polynucleotide encoding the split intein N-fragment of this disclosure, or the split intein C-fragment of this disclosure, or any one of the complexes of this disclosure wherein the compound of interest is a polypeptide or protein and the linker, if present, is a peptide linker.

In another aspect, this disclosure relates to a vector comprising the polynucleotide of this disclosure.

In another aspect, this disclosure relates to a host cell comprising the polynucleotide or the vector of this disclosure.

In another aspect, this disclosure relates to a composition comprising the first complex of this disclosure and the second complex of this disclosure.

In another aspect, this disclosure relates to a method to obtain a conjugate between a first compound of interest and a second compound of interest comprising
(i) contacting
   (a) the first complex of this disclosure, wherein the complex comprises the first compound of interest and a split intein N-fragment comprising the amino sequence of SEQ ID NO: 1 or a functionally equivalent variant thereof having at least 90% sequence identity with SEQ ID NO: 1, or an amino acid sequence selected from the group consisting of SEQ ID NO: 103-110 with
   (b) the second complex of this disclosure, wherein the complex comprises the second compound of interest and a split intein C-fragment comprising the amino acid sequence of SEQ ID NO: 7 or a functionally equivalent variant thereof having at least 88% sequence identity with SEQ ID NO: 7, or an amino acid sequence selected from the group consisting of SEQ ID NO: 114-120
      or
      a complex comprising an AceL-TerL split intein C-fragment or a functionally equivalent variant thereof and the second compound of interest, wherein the complex optionally comprises a linker between the split intein C-fragment and the second compound of interest and wherein
         the second compound of interest is bound to the C-terminus of the split intein C-fragment by an amide linkage or
         if the complex comprises a linker, the second compound of interest is bound to the linker by an amide linkage and/or the linker is bound to the C-terminus of the split intein C-fragment by an amide linkage
   under appropriate conditions for binding the split intein N-fragment to the split intein C-fragment to form an intein intermediate and
(ii) allowing the intein intermediate to react to form a conjugate between the first and the second compound of interest.

In another aspect, this disclosure relates to a method to obtain a conjugate between a first compound of interest and a second compound of interest comprising
(i) contacting
   (a) the first complex of this disclosure, wherein the complex comprises the first compound of interest and a split intein N-fragment comprising the amino sequence of SEQ ID NO: 1 or a functionally equivalent variant thereof having at least 90% sequence identity with SEQ ID NO: 1, or an amino acid sequence selected from the group consisting of SEQ ID NO: 103-110
      or
      a complex comprising the second compound of interest and an AceL-TerL split intein N-fragment or a functionally equivalent variant thereof, wherein the complex optionally comprises a linker between the compound of interest and the split intein N-fragment, and wherein
         the compound of interest is linked to the N-terminus of the split intein N-fragment by an amide linkage or
         if the complex comprises a linker, the compound of interest is bound to the linker by an amide linkage and/or the linker is bound to the N-terminus of the split intein N-fragment by an amide linkage.
      with
   (b) the second complex of this disclosure, wherein the complex comprises the second compound of interest and a split intein C-fragment comprising the amino acid sequence of SEQ ID NO: 7 or a functionally equivalent variant thereof having at least 88% sequence identity with SEQ ID NO: 7, or an amino acid sequence selected from the group consisting of SEQ ID NO: 114-120
   under appropriate conditions for binding the split intein N-fragment to the split intein C-fragment to form an intein intermediate and
(ii) allowing the intein intermediate to react to form a conjugate between the first and the second compound of interest.

In another aspect, this disclosure relates to a method to obtain a conjugate of a compound of interest with a nucleophile comprising
(i) contacting
   (a) the first complex of this disclosure, wherein the split intein N-fragment comprises the amino acid sequence of SEQ ID NO: 1 or a functionally equivalent variant thereof having at least 90% sequence identity with SEQ ID NO: 1 or an amino acid sequence selected from the group consisting of SEQ ID NO: 103-110
   or
   a complex comprising a compound of interest and an AceL-TerL split intein N-fragment or a functionally equivalent variant thereof, wherein the complex optionally comprises a linker between the compound of interest and the split intein N-fragment, and wherein
      the compound of interest is linked to the N-terminus of the split intein N-fragment by an amide linkage or
      if the complex comprises a linker, the compound of interest is bound to the linker by an amide linkage and/or the linker is bound to the N-terminus of the split intein N-fragment by an amide linkage.
   with
   (b) a split intein C-fragment comprising an amino acid sequence selected from the group consisting of SEQ ID NO: 8, 9, 23-48 and 141-166, under appropriate conditions for binding between the split intein N-fragment and the split intein C-fragment to form an intein intermediate and
(ii) contacting the intein intermediate with an exogenous nucleophile.

In another aspect, this disclosure relates to a composition comprising:
(a) a first polynucleotide encoding a first fusion protein comprising, from the N-terminus to the C-terminus:
   a first polypeptide of interest and
   a split intein N-fragment comprising the sequence of SEQ ID NO: 1 or a variant thereof having at least 90% sequence identity with SEQ ID NO: 1, or an amino acid sequence selected from the group consisting of SEQ ID NO: 103-110 and
(b) a second polynucleotide encoding a second fusion protein comprising, from the N-terminus to the C-terminus:
   an AceL-TerL split intein C-fragment or a variant thereof or a split intein C-fragment comprising the sequence of SEQ ID NO: 7 or a variant thereof having at least 88% sequence identity with SEQ ID NO: 7 or an amino acid sequence selected from the group consisting of SEQ ID NO: 114-120 and
   a second polypeptide of interest
or
(a) a first polynucleotide encoding a first fusion protein comprising, from the N-terminus to the C-terminus:
   a first polypeptide of interest and
   an AceL-TerL split intein N-fragment or a variant thereof, or a split intein N-fragment comprising the sequence of SEQ ID NO: 1 or a variant thereof having at least 90% sequence identity with SEQ ID NO: 1, or an amino acid sequence selected from the group consisting of SEQ ID NO: 103-110 and
(b) a second polynucleotide encoding a second fusion protein comprising, from the N-terminus to the C-terminus:
   a split intein C-fragment comprising the sequence of SEQ ID NO: 7 or a variant thereof having at least 88% sequence identity with SEQ ID NO: 7 or an amino acid sequence selected from the group consisting of SEQ ID NO: 114-120 and
   a second polypeptide of interest.

In another aspect, this disclosure relates to a method for expressing a gene of interest in a cell comprising:
(i) contacting the cell with
   (a) a first polynucleotide encoding a first fusion protein comprising, from the N-terminus to the C-terminus:
      a first polypeptide of interest and
      a split intein N-fragment comprising the sequence of SEQ ID NO: 1 or a functionally equivalent variant thereof having at least 90% or an amino acid sequence selected from the group consisting of SEQ ID NO: 103-110, and
   (b) a second polynucleotide encoding a second fusion protein comprising, from the N-terminus to the C-terminus:
      an AceL-TerL split intein C-fragment or a functionally equivalent variant thereof, or a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
      second polypeptide of interest,
   or
   (a) a first polynucleotide encoding a first fusion protein comprising, from the N-terminus to the C-terminus:
      a first polypeptide of interest and
      an AceL-TerL split intein N-fragment or a functionally equivalent variant thereof, or a split intein N-fragment comprising the sequence of SEQ ID NO: 1 or a functionally equivalent variant thereof having at least 90% or an amino acid sequence selected from the group consisting of SEQ ID NO: 103-110, and
   (b) a second polynucleotide encoding a second fusion protein comprising, from the N-terminus to the C-terminus:
      a split intein C-fragment comprising the sequence of SEQ ID NO: 7 or a variant thereof having at least 88% sequence identity with SEQ ID NO: 7, or an amino acid sequence selected from the group consisting of SEQ ID NO: 114-120, and a
      second polypeptide of interest,
(ii) allowing the expression of the first and the second polynucleotides so that the first and the second fusion proteins are produced and
(iii) allowing the contact between the first and second fusion proteins so that the split intein N-fragment binds to the split intein C-fragment to form a intein intermediate and the intein intermediate reacts to covalently link the C-terminus of the first polypeptide of interest to the N-terminus of the second polypeptide of interest.

In another aspect, this disclosure relates to a method for expressing a gene of interest comprising:
(i) contacting a first cell with a first polynucleotide encoding a first fusion protein comprising, from the N-terminus to the C-terminus:
   a first polypeptide of interest and
   a split intein N-fragment comprising the sequence of SEQ ID NO: 1 or a functionally equivalent variant thereof having at least 90% or an amino acid sequence selected from the group consisting of SEQ ID NO: 103-110,
wherein the first fusion protein comprises a signal peptide, and
(ii) contacting a second cell with a second polynucleotide encoding a second fusion protein comprising, from the N-terminus to the C-terminus:
   an AceL-TerL split intein C-fragment or a functionally equivalent variant thereof, or a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
   second polypeptide of interest
wherein the second fusion protein comprises a signal peptide,
or
(i) contacting a first cell with a first polynucleotide encoding a first fusion protein comprising, from the N-terminus to the C-terminus:
   a first polypeptide of interest and
   an AceL-TerL split intein N-fragment or a functionally equivalent variant thereof, or a split intein N-fragment comprising the sequence of SEQ ID NO: 1 or a functionally equivalent variant thereof having at least 90% or an amino acid sequence selected from the group consisting of SEQ ID NO: 103-110,
wherein the first fusion protein comprises a signal peptide, and
(ii) contacting a second cell with a second polynucleotide encoding a second fusion protein comprising, from the N-terminus to the C-terminus:
   a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
   second polypeptide of interest
wherein the second fusion protein comprises a signal peptide,
1. allowing the expression of the first and the second polynucleotides so that the first and the second fusion proteins are produced and secreted,
2. allowing the contact between the first and second fusion proteins so that the split intein N-fragment binds to the split intein C-fragment to form a intein intermediate and the intein intermediate reacts to covalently link the C-terminus of the first polypeptide of interest to the N-terminus of the second polypeptide of interest.

### BRIEF DESCRIPTION OF THE FIGURES

FIG. 1. (A)-(E) RP-HPLC analysis of inteins utilized in this study. The masses corresponding to each RP-HPLC chromatogram are reported in Table 3.

FIG. 2. (A)-(D) Representative splicing gels of protein trans-splicing reactions. (A) Representative SDS-PAGE gels of protein trans-splicing reactions for Cat and AceL* at the indicated temperatures. Bands correspond to MBP-Int^{N} (N), Int^{C}-GFP (C) and the spliced product (SP) are indicated. (B) Representative SDS-PAGE gels of protein trans-splicing reactions for Cat and AceL* at the indicated concentrations of urea. Bands corresponding to MBP-Int^{N} (N), Int^{C}-GFP (C) and the spliced product (SP) are indicated. (C) Representative SDS-PAGE gels of protein trans-splicing reactions for Cat with the indicated −1 and −2 N-extein mutations (from the WT “FE” sequence). Bands corresponding to MBP-Cat^{N} (N), Cat^{C}-GFP (C) and the spliced product (SP) are indicated. C-terminal cleavage is observed for the −1A and −1P mutations and are indicated on the gel (GFP). (D) Representative SDS-PAGE gels of protein trans-splicing reactions for Cat with the indicated +2 and +3 C-extein mutations (from the WT “EF”). Bands corresponding to MBP-Cat^{N} (N), Cat^{C}-GFP (C) and the spliced product (SP) are indicated.

FIG. 3. (A)-(B) Reaction progress curves. (A) and (B) Reaction progress curves are presented for the splicing reactions carried out in this study. The best-fit lines for each reaction are shown.

FIG. 4. (A)-(D) Expression of Atypical Split Inteins. Lanes correspond to (W) the whole cell lysate, (P) the inclusion body pellet, (S) the soluble fraction of the lysate, (FT) flow through of the soluble lysate batch bound to Ni-NTA affinity beads, (E) a 3 CV elution of 250 mM imidazole. (A) Purification of SUMO-GOSH, SUMO-AceL*^{C}, and SUMO-Cat^{C} from E. coli expression (18° C., 16 h). (B) Purification of SUMO-GOSH, SUMO-AceL*^{C}-Sumo, and SUMO-Cat^{C} from E. coli expression (37° C., 3 hours). (C) Purification of SUMO-GOS^{N}, SUMO-AceL*^{N}, and SUMO-Cat^{N} from E. coli expression (37° C., 3 hours). (D) Purification of GOSH-GFP, AceL*^{C}-GFP, and Cat^{C}-GFP from E. coli expression (18° C., 16 hours).

FIG. 5. (A)-(D) Characterization of a consensus atypical (Cat) split intein. (A) Pairwise sequence alignment of Cat and AceL* highlighting identical (black) and similar (gray) residues. (B) Reaction progress curve for Cat splicing at 30° C. (C) Splicing rates for Cat and AceL* as a function of temperature (n=3, error=SEM). AceL* is inactive at 50° C. (D) Splicing rates for Cat and AceL* as a function of added Urea (n=3, error=SEM). AceL* is not active in the presence of 2 M and 4 M Urea (NA).

FIG. 6. (A)-(D) Structural effects of Cat fragment association. (A)¹H-¹⁵N HSQC spectra of 15N labeled Cat^{N} in free from (black) and in complex with unlabeled Cat^{C} (gray). (B) 1H-15N HSQC spectra of 15N labeled Cat^{C} in free form (black) and in complex with unlabeled Cat^{N} (gray). (C) Far UV circular dichroism spectra of Cat^{N} (black), Cat^{C} (dark gray) and the Cat^{N}+Cat^{C} complex (light gray). (D) Size exclusion chromatograms of Cat^{N} (black), Cat^{C} (dark gray), and the Cat^{N}+Cat^{C} complex (light gray).

FIG. 7. (A)-(C) Disorder to order transition of Cat^{N} (A) (¹⁵N-¹H) heteronuclear NOE of Cat^{N} in the presence of Cat^{C} (left) and in free form (right). (B) Spin-spin relaxation rate of Cat^{N} in the presence of Cat^{C} (left) and in free form (right). (C) Perturbation of Cα and Cβ chemical shifts of Cat^{N} in the presence of Cat^{C} (left) and in free form (right). Δδ(Cα,Cβ)=(δCβ−δCα)Observed−(δCβ−δCα)Random Coil.

FIG. 8. (A)-(C) Solution NMR structure of Cat. (A) Backbone conformation of the 20 lowest energy conformers obtained in the structure calculation of the Cat^{N} (dark)—Cat^{C} (light) split intein complex. The Cat^{C} solubility tag is rendered in transparent gray. Structures are shown with a 180° rotation (top and bottom renderings). (B) Cartoon depiction of the lowest energy conformer. Structures are shown with a 180° rotation (top and bottom renderings). (C) Zoom view of the Cat active site with Ala₁, Ser₇₅, His₇₈, and Hisi₃₃ depicted as sticks. The distances between the carbonyl oxygen of Ala₁ and amide and hydroxyl protons of Ser₇₅ are indicated.

FIG. 9. (A)-(C) Structure of Cat Complex. (A) Average per residue Root Mean Square Deviation (RMSD) from average structure for 20 least energy conformers of Cat^{N}-Cat^{C} complex obtained in NMR structure calculation. (B) Average per residue RMSD plotted against residue number for Cat^{N} (gray)—Cat^{C} (black) complex. Extein regions are marked with a gray and the solubility tag used with Cat^{C} is shown as dashed lines. (C) Sequence logo of the Block B loop (left) Block F loop (middle) and C-terminal Block G (right) generated from an alignment of TerL intein homologues (Table 1).

FIG. 10. (A)-(C) Localization of Disorder in the Cat Fragments. (A) RP-HPLC chromatogram stack from the limited proteolysis of Cat^{N} (left), Cat^{C} (middle) and a 1:1 Cat^{N}+Cat^{C} complex (right) with samples quenched after the indicated times. (B) Sequence of Cat with the disordered regions of Cat^{C} highlighted in dark gray and the protected center highlighted in light gray. (C) Model of Cat disorder mapped onto the NMR structure with the N-intein highlighted in light gray, disordered region of Cat^{C} highlighted in dark gray, and the protected center highlighted in medium gray. A zoom view of the active site is shown with the splicing residues rendered as sticks.

FIG. 11. (A)-(B) RP-HPLC analysis of limited Proteolysis of Cat fragments. (A) RP-HPLC from the Cat^{N} (left) and Cat^{C} (right) proteolysis experiment (t=30 min) with numbered samples corresponding to the ESI-MS data in Table 8. (B) Primary sequence of the Cat^{N} and Cat^{C} inteins used in the limited proteolysis experiment with the proteolysis fragments detected indicated below as brackets. The number of each bracket corresponds to the RP-HPLC peak in panel A.

FIG. 12. (A)-(D) Hydrophobic residues drive Cat association. (A) Surface rendering of Cat^{N} with hydrophobic residues colored in grayscale based on the normalized consensus hydrophobicity scale. Cat^{C} is depicted as a cartoon. (B) Surface rendering of Cat^{C} with hydrophobic residues in grayscale. Cat^{N} is depicted as a cartoon. (C) Equilibrium fluorescence anisotropy measurements of FI-Cat^{N} (500 pM) in the presence of SUMO-Cat^{C} (indicated concentration) in low (100 mM NaClblack) and high (500 mM NaClgray dashed) salt buffers. (D) Concentration dependence of the observed rates of FI-Cat^{N}+SUMO-Cat^{C} association in low (100 mM NaClblack) and high (500 mM NaClgray dashed) salt buffers.

FIG. 13. (A)-(C) Electrostatic surface of Cat. (A) Electrostatic surface potential of Cat^{N} with electronegative regions colored in smooth grayscale, electropositive regions colored in textured grayscale, and neutral regions colored in white. Cat^{C} is depicted as a cartoon. (B) Electrostatic surface potential of Cat^{C} with electronegative regions colored in smooth grayscale, electropositive regions colored in textured grayscale, and neutral regions colored in white. Cat^{N} is depicted as a cartoon. (C) Representative data and fits for kinetic binding experiments. Top: Single (left) and double (right) exponential models for the nonlinear least squares fitting of stopped flow anisotropy measurements of FI-Cat^{N} upon mixing with SUMO-Cat^{C}. Bottom: Residual values obtained between experimental and predicted values are plotted for the single (left) and double (right) exponential fits.

FIG. 14. (A)-(E) Extein Dependence of Cat. (A) Schematic of the assay used to investigate the impact of local extein sequences on Cat splicing. An N-extein maltose binding protein (MBP) is fused to Cat^{N} while a C-extein green fluorescent protein (GFP) is fused to Cat^{C}. The native extein sequences (Phe₋₂, Glu₋₁, Glu₊₂, Phe₊₃) are shown within these fusion proteins. (B) Splicing rates for Cat in the presence of non-native C-extein residues (n=3, error=SEM). Each indicated value corresponds to a single point mutation within the C-extein from the wild type (WT) sequence. (C) Splicing rates for Cat in the presence of non-native N-extein residues (n=3, error=SEM). Each indicated value corresponds to a single point mutation within the N-extein from the wild type (WT) sequence. (D) Zoom view of the Cat active site with Cys₊₁, Glu₊₂, Asp₁₁₅, Asn₁₂₃, His₁₃₃, and Ala₁₃₄ depicted as sticks. (E) Zoom view of Cat active site with Glu₋₁, Ala₁, Ser₇₅, and His₇₈ depicted as sticks.

### DETAILED DESCRIPTION

The present disclosure relates to the provision of new atypical split inteins and its uses in biochemical engineering.

### Split Intein N-Fragments

In a first aspect this disclosure relates to a split intein N-fragment comprising the amino acid sequence of SEQ ID NO: 1 or a variant thereof having at least 90% sequence identity with SEQ ID NO: 1.

As used herein, the term “intein” means a naturally-occurring or artificially-constructed polypeptide sequence capable of catalyzing a protein splicing reaction that excises the intein sequence from a precursor protein and joins the flanking sequences (N- and C-exteins) with a peptide bond. They are typically 150-550 amino acids in size and may also contain a homing endonuclease domain. A list of known inteins is published on the world wide web at inteins.biocenter.helsinki.fi/.

The terms “polypeptide”, “peptide” or “protein” are used interchangeably herein to refer to polymers of amino acids of any length.

The term “amino acid” refers to naturally occurring and synthetic amino acids, as well as amino acid analogs and amino acid mimetics that function in a manner similar to the naturally occurring amino acids. Furthermore, the term “amino acid” includes both D- and L-amino acids (stereoisomers).

The term “natural amino acids” or “naturally occurring amino acid” comprises the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine.

As used herein the term “non-natural amino acid” or “synthetic amino acid” refers to a carboxylic acid, or a derivative thereof, substituted with an amine group and being structurally related to a natural amino acid. Illustrative non-limiting examples of modified or uncommon amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxy lysine, alio hydroxy lysine, 3-hydroxyproline, 4-hydroxyproline, isodesmosine, alloisoleucine, N-methylglycine, N-methyliso leucine, 6-N-methyl-lysine, N-methylvaline, norvaline, norleucine, ornithine, etc. This group also includes the D-isomers of the “natural amino acids”.

The term “split intein” as used herein refers to any intein in which the N-terminal and C-terminal amino acid sequences are not directly linked via a peptide bond, such that the N-terminal and C-terminal sequences become separate fragments that can non-covalently re-associate, or reconstitute, into an intein that is functional for trans-splicing reactions.

As used herein, the term “split intein N-fragment” or “N-terminal split intein” or “N-terminal intein fragment” or “N-terminal intein sequence” (abbreviated “Int N”)” refers to any intein sequence that comprises an N-terminal amino acid sequence that is functional for trans-splicing reactions, that is, that is capable of associating with a functional split intein C-fragment to form a complete intein that is capable of excising itself from the host protein, catalyzing the ligation of the extein or flanking sequences with a peptide bond, or that upon association with a split intein C-fragment catalyzes the “N-terminal cleavage”, that is, the nucleophilic attack of the peptide bond between the extein and the N-terminus of the split intein N-fragment resulting in the breaking of said peptide bond.

It must be noted that as used herein and in the appended claims, the singular forms “a,” “an,” and “the” include plural referents unless the context clearly dictates otherwise. Thus, for example, reference to “a split intein” includes a plurality of such split inteins and reference to “the polypeptide” includes reference to one or more polypeptides and equivalents thereof known to those skilled in the art, and so forth.

In certain embodiments, the split intein N-fragment comprises the amino acid sequence of SEQ ID NO: 1. The split intein N-fragment can comprise additional amino acid residues linked to the N- and/or C-terminus of the sequence of SEQ ID NO: 1. In certain embodiments, the split intein N-fragment comprises less than 10, less than 9, less than 8, less than 7, less than 6, less than 5, less than 4, less than 3, less than 2, or 1 additional amino acid residues linked to the N- and/or C-terminus of the sequence of SEQ ID NO: 1. In another embodiment, the split intein N-fragment consists on the amino acid sequence of SEQ ID NO: 1.

In certain embodiments, the split intein N-fragment comprises or consists of a variant of the amino acid sequence of SEQ ID NO: 1 having at least 90% sequence identity with SEQ ID NO: 1.

The term “variant” as used herein refers to a polypeptide molecule that is substantially similar to a particular polypeptide sequence. The variant may be similar in structure and biological activity to the polypeptide from which it derives. Thus, the variant may refer to a mutant of a polypeptide sequence. The term “mutant” refers to a polypeptide molecule the sequence of which has one or more amino acids added, deleted, substituted or otherwise chemically modified in comparison to the polypeptide molecule from which it derives. The mutant may retain substantially the same properties as the polypeptide molecule from which it derives or lack the biological activity of the claimed sequences.

The variant of the split intein N-fragment of SEQ ID NO: 1 has at least 90% sequence identity with SEQ ID NO: 1. In certain embodiments, the variant of the split intein N-fragment of SEQ ID NO: 1 has at least 91%, at least 92%, at least 93%, at least 94%, at least 95%, at least 96%, at least 97%, at least 98%, at least 99% sequence identity with SEQ ID NO: 1.

In certain embodiments of this aspect of the present disclosure, the variant of the split intein N fragment of SEQ ID NO: 1 has a length of between 14 and 60 amino acids, for example, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 amino acids.

The terms “identity”, “identical”, “percent identity” or “sequence identity” in the context of two or more amino acid or nucleotide sequences, refer to two or more sequences or subsequences that are the same or have a specified percentage of amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid sequences. One such non-limiting example of a sequence alignment algorithm is the algorithm described in Karlin et al., 1990, Proc. Natl. Acad. Sci., 87:2264-8, as modified in Karlin et al., 1993, Proc. Natl. Acad. Sci., 90:5873-7, and incorporated into the N BLAST and XBLAST programs (Altschul et al., 1991, Nucleic Acids Res., 25:3389-402). In certain embodiments, Gapped BLAST can be used as described in Altschul et al., 1997, Nucleic Acids Res. 25:3389-402. BLAST-2, WU-BLAST-2 (Altschul et al., 1996, Methods in Enzymology, 266:460-80), ALIGN, ALIGN-2 (Genentech, South San Francisco, Calif.) or Megalign (DNASTAR) are additional publicly available software programs that can be used to align sequences. In certain alternative embodiments, the GAP program in the GCG software package, which incorporates the algorithm of Needleman and Wunsch (J. Mol. Biol. 48:444-53 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between amino acid sequences is determined using the algorithm of Myers and Miller (CABIOS, 4:1 1-7 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second amino acid sequence is calculated as 100× (Y/Z), where Y is the number of amino acid residues scored as identical matches in the alignment of the first and second sequences (as aligned by visual inspection or a particular sequence alignment program) and Z is the total number of residues in the second sequence. If the second sequence is longer than the first sequence, then the global alignment taken the entirety of both sequences into consideration is used, therefore all letters and null in each sequence must be aligned. In this case, the same formula as above can be used but using as Z value the length of the region wherein the first and second sequence overlaps, said region having a length which is substantially the same as the length of the first sequence.

As a non-limiting example, whether any particular polypeptide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 5371 1). Bestfit uses the local homology algorithm of Smith and Waterman, Advances in Applied Mathematics 2:482-9 (1981), to find the best segment of homology between two sequences. When using Bestfit or any other sequence alignment program to determine whether a particular sequence is, for instance, 95% identical to a reference sequence according to the present disclosure, the parameters are set such that the percentage of identity is calculated over the full length of the reference amino acid sequence and that gaps in homology of up to 5% of the total number of nucleotides in the reference sequence are allowed.

In certain embodiments, the variant of the split intein N-fragment of SEQ ID NO: 1 has at least 90% sequence identity with SEQ ID NO: 1 over the whole length of the sequence.

In certain embodiments, the variant of the split N-intein fragment of SEQ ID NO: 1 comprises or consists of an amino acid sequence selected from the group consisting of SEQ ID NO: 2-6, and 125-127.

In another embodiment, the variant of the split N-intein fragment of SEQ ID NO: 1 is a functionally equivalent variant of SEQ ID NO: 1.

The term “functionally equivalent variant” as used herein is understood to mean all those proteins derived from a sequence by modification, insertion and/or deletion or one or more amino acids, whenever the function is substantially maintained, particularly in the case of a functionally equivalent variant of the split intein N-fragment refers to maintaining its activity.

In certain embodiments, the functionally equivalent variant of the split intein N-fragment of SEQ ID NO: 1 maintains or improves the activity from the split intein N-fragment of SEQ ID NO: 1.

The term “activity” as used herein referring to the split intein N-fragment, refers to the ability of the split intein N-fragment to bind to a split intein C-fragment and catalyze the “N-terminal cleavage”, that is, the nucleophilic attack of the peptide bond between the extein and the N-terminus of the split intein N-fragment, resulting in the breaking of said peptide bond. The activity of the split intein N-fragment can also refer to the “trans-splicing activity”, which is understood as the ability of said split intein N-fragment to bind to a functional split intein C-fragment excising the complete intein from the host protein, catalyzing the ligation of the extein or flanking sequences with a peptide bond. The activity is dependent on reaction conditions, including temperature, pH and the presence of chaotropic agents. The commonly used unit is t_{1/2}, which represents the time at which half of the catalyzed reaction has been completed. Additionally, intein activity is also measured by the rate constant (k) of the catalyzed reaction, that is, how many times per second does the reaction take place.

Suitable assays for determining whether a polypeptide is a functionally equivalent variant of a given split N-intein, in terms of its trans-splicing activity, include splicing assays, such as those described for example in the methods of the present application or disclosed in Shah N H et al (Shah N H et al., 2012, J Chem Soc, vol 134, 11338), as long as in these assays the split intein N-fragment is combined with a functional split intein C-fragment, that is a split intein C-fragment which is capable of catalyzing “C-terminal cleavage”. The assays described above allow to determine and characterize trans-splicing reactions in which functional N and C-intein fragments bind to each other and subsequently carry out a reaction by which they excise themselves out and form a new peptide bond between the N and C-exteins. Other assays have been developed, which rely on the use of functional N-intein and a C-intein mutant that prevents trans-splicing, so that the reaction is stopped after the cleavage of the N-extein from the N-intein. Such assays (Vila-Perelló et al. J Am Cem Soc. 2013, 135(1): 286-292) allow to characterize the ability of an N-intein to perform the N-terminal cleavage reaction. Additionally, other assays exist to measure the affinity between N and C-terminal inteins (Shah et al. Angew Chem Int Ed Engl. 2011, 50(29): 6511-5).

According to the present disclosure, the activity of the split N-intein of this disclosure is substantially maintained if the functionally equivalent has at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of its activity. Furthermore, the activity of the split N-intein of this disclosure is substantially improved if the functionally equivalent variant has at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, or at least 65%, at least 70%, at least 75%, at least 80%, at least 85%, at least 90%, at least 95%, at least 100%, at least 150%, at least 200%, at least 300%, at least 400%, at least 500%, at least 1000%, or more of its activity.

As mentioned above, the activity of the split N-intein of this disclosure depends on a number of reaction parameters, including temperature, chaotropic environment and pH. Thus, in one embodiment, the functionally equivalent variant of the split intein N-fragment of this disclosure maintains or improve its activity at a temperature of at least 0° C., at least 5° C., at least 10° C., at least 15° C., at least 20° C., at least 25° C., at least 30° C., at least 35° C., at least 37° C., at least 40° C., at least 45° C., at least 50° C., at least 55° C., at least 60° C., at least 65° C., at least 70° C. or higher; in certain embodiments at a temperature of 50° C. Likewise, in another embodiment the functionally equivalent variant of the split N-intein of this disclosure maintains or improves its activity at least at pH 2.0, or at least at pH 2.5, or at least at pH 3.0, or at least at pH 3.5, or at least at pH 4.0, or at least at pH 4.5, or at least at pH 5.0, or at least at pH 5.5, or at least at pH 6.0, or at least at pH 6.5, or at least at pH 7.0, or at least at pH 7.2, or at least at pH 7.5, or at least at pH 8.0, or at least at pH 8.5, or at least at pH 9.0, or at least at pH 9.5, or at least at pH 10.0, or at least at pH 10.5, or at least at pH 11.0, or at least at pH 11.5, or at least at pH 12.0, or at least at pH 12.5, or at least at pH 13.0, or at least at pH 13.5, or at least at pH 14; in certain embodiments at pH 7.2. In another embodiment, the functionally equivalent variant of the split N-intein of this disclosure maintains or improves its activity at urea 1 M, or at least at urea 1.5 M, or at urea least 2 M, or at least urea 3 M, or at least urea 3.5 M, or at least urea 4 M, or at least urea 4.5 M, or at least urea 5 M; in certain embodiments at urea 2 M or at urea 4 M. In certain embodiments, the functionally equivalent variant of the split N-intein of this disclosure maintains or improves its activity at urea 2 M or urea 4 M. In certain embodiments, the functionally equivalent variant of the split N-intein of this disclosure maintains or improves its at a temperature of 50° C., at pH 7.2 and at urea 2 M or urea 4 M. All possible combinations of temperatures, urea concentration, other denaturants and pH are also contemplated by this disclosure.

In certain embodiments, the functionally equivalent variant of the split intein N-fragment of this disclosure that maintains or improves its activity has at least 90%, at least 91%, at least 92%, at least 93%, at least 94%, at least 95%, at least 96%, at least 97%, at least 98% or at least 99% sequence identity with SEQ ID NO: 1.

In another embodiment, the functionally equivalent variant of the split intein N-fragment of SEQ ID NO: 1 comprises or consist of the amino acid sequence of SEQ ID NO: 4 or SEQ ID NO: 125.

### Complex Comprising a Split Intein N-Fragment

In another aspect, this disclosure relates to a complex, hereinafter first complex of this disclosure, comprising:

(i) a compound of interest,
(ii) the split intein N-fragment of this disclosure, or a split intein N-fragment comprising the amino acid sequence selected from the group consisting of SEQ ID NO: 103-110,
wherein the complex optionally comprises a linker between (i) and (ii) and
wherein
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.

As used herein, the term “compound of interest” include any synthetic or naturally occurring molecule, including a protein or peptide, a single or doubled stranded oligonucleotide, small molecule a drug or a cytotoxic molecule. The term therefore encompasses those compounds traditionally regarded as drugs, vaccines, and biopharmaceuticals including molecules such as proteins, peptides, and the like. Examples of therapeutic agents are described in well-known literature references such as the Merck Index (14th edition), the Physicians' Desk Reference (64th edition), and The Pharmacological Basis of Therapeutics (1st edition), and they include, without limitation, medicaments; substances used for the treatment, prevention, diagnosis, cure or mitigation of a disease or illness; substances that affect the structure or function of the body, or pro-drugs, which become biologically active or more active after they have been placed in a physiological environment. In addition, the “compound of interest” may include any non-protein molecule having a carboxylic group able to bind the amino-terminus end of the N-intein.

Optionally, the compound of interest and the split intein N-fragment may be joined through a linker, so the linker is located in between the compound of interest and the N-intein. The nature of the linker will depend on the nature of the compound of interest. In certain embodiments, the linker is a peptide. In certain embodiments, the linker is a peptide having a length of 1, 2, 3, 4, 5, 10, 20, 50, 100 or more amino acid residues; specifically, it may be 1 to 3 amino acid residues. If the compound of interest is a peptide or protein, the N-terminus of the linker is linked to the C-terminus of the compound of interest and the C-terminus of the linker is linked to the N-terminus of the N-intein through peptide bonds.

In certain embodiments, the linker is a non-peptide linker. Non-peptide linkers are for example, alkyl linkers such as —HN—(CH₂)s —CO—, wherein s=2-20 can be used. These alkyl linkers may further be substituted by any non-sterically hindering group such as lower alkyl (e.g., Ci-Ce), halogen (e.g., CI, Br), CN, NH2, phenyl, etc.

Another type of non-peptide linker is a polyethylene glycol group, such as: —HN-(CH2)2-(O-CH2-CH2)n-O-CH2-CO, wherein n is such that the overall molecular weight of the linker ranges from approximately 101 to 5000; in certain embodiments 101 to 500.

In another embodiment, the non-peptide linker comprises a basic nucleotide, polyether, polyamine, polyamide, carbohydrate, lipid, polyhydrocarbon, or other polymeric compounds.

In certain embodiments, the complex does not comprise a linker between the compound of interest and the split intein N-fragment. In this embodiment, the compound of interest is linked to the N-terminus of the split intein N-fragment by an amide linkage.

In certain embodiments, the complex comprises a linker between the compound of interest and the split intein N-fragment. In this embodiment, the compound of interest may be bound to the linker by any suitable means, depending on the chemical nature of the compound of interest and of the linker. In this embodiment, the linker is bound to the N-terminus of the split intein N-fragment by an amide linkage. In a another embodiment, the compound of interest is bound to the linker by an amide linkage, in which case the linker may be found to the N-terminus of the split intein N-fragment by any suitable means. In another embodiment, the compound of interest is bound to the linker by a amide linkage and the linker is bound to the N-terminus of the split intein N-fragment by an amide linkage.

In another embodiment, the compound of interest is a protein having the C-terminal amino acid residues of the extein capable of being spliced by an intein comprising the N-intein of SEQ ID NO: 1. In another embodiment, the compound of interest is a protein having the sequence Glu-Phe-Glu in its C-terminus. In another embodiment, the compound of interest is a protein having the sequence Phe-Glu in its C-terminus. In another embodiment, the compound of interest is a protein having the residue Glu in its C-terminus.

In another embodiment, when the compound of interest is not a protein, the N-intein comprises or consists on the polypeptide of SEQ ID NO: 4-6, 125-127 or 168-170. In another embodiment, when the compound of interest is not a protein, the compound of interest and the N-intein are joined through a linker in which case, the linker is a peptide having the C-terminal amino acid residues of the extein capable of being spliced by an intein comprising the split intein N-fragment of sequence SEQ ID NO: 1; in certain embodiments, the linker is a peptide having the sequence Glu-Phe-Glu, Phe-Glu or Glu in its C-terminus.

In another embodiment, the compound of interest is a protein that does not have the C-terminal amino acid residues of the extein capable of being spliced by an intein comprising the split intein N-fragment of SEQ ID NO: 1, in which case (i) the N-intein comprises or consists on the polypeptide of sequence SEQ ID NO: 4-6, 125-127 or 168-170 or (ii) the compound of interest and the N-intein are joined through a linker in which case, the linker is a peptide having the C-terminal amino acid residues of the extein capable of being spliced by an intein comprising the split intein N-fragment of SEQ ID NO: 1; in certain embodiments, the linker is a peptide having the sequence Glu-Phe-Glu, Phe-Glu or Glu in its C-terminus.

The phrase “peptide bond” refers to a covalent chemical bond —CO—NH— formed between two molecules when the carboxy part of one molecule, referred to as a carboxy component, reacts with the amino part of another molecule, referred to as an amino component, causing the release of a molecule. For example, proteinogenic L-amino acids can form the peptide bond upon joining with the release of a molecule of water. Therefore, proteins and peptides can be regarded as chains of amino acid residues held together by peptide bonds. A peptide bond is an “amide bond” or “amide linkage”.

In certain embodiments, the compound of interest is a protein or polypeptide.

In another embodiment, the compound of interest is a protein of more than 25 KDa, more than 50 KDa or more than 100 KDa.

In certain embodiments, the protein is Cas9, or a fragment of Cas9. The term “Cas9” or “CRISPR-associated endonuclease Cas9”, as used herein, refers to a protein, which is the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with a sgRNA can introduce site-specific double strand breaks (DSBs) into genomic DNA of live cells from various organisms. The Cas9 can be of any origin, including for example, Streptocccus thermophilus, Streptococcus pyogenes, Staphylococcus aeureus, Francisella tularensis, Actinomyces naeslundii, Neiserria meningitides, Listeria innocua, among others. In certain embodiments, the term “Cas9” refers to any one of the proteins defined by the UniProtKB/Swiss-Prot accession numbers G3ECR1 (entry version 31 of 10 Apr. 2019, sequence version 2 of 13 Jun. 2012), Q99ZW2 (entry version 112 of 31 Jul. 2019, sequence version 1 of 1 Jun. 2001), J7RUA5 (entry version 33 of 8 May 2019, sequence version 1 of 31 Oct. 2012), A0Q5Y3 (entry version 62 of 16 Jan. 2019, sequence version 1 of 9 Jan. 2007), J3F2B0 (entry version 33 of 8 May 2019, sequence version 1 of 3 Oct. 2012), Q03JI6 (entry version 70 of 8 May 2019, sequence version 1 of 14 Nov. 2006), C9X1G5 (entry version 47 of 31 Jul. 2019, sequence version 1 of 24 Nov. 2009), Q927P4 (entry version 94 of 8 May 2019, sequence version 1 of 1 Dec. 2001).

In certain embodiments, the compound of interest of the complex is a polypeptide or protein, and if the complex comprises a linker, the linker is a peptide linker. In this embodiment, the complex is a fusion protein.

The term “fusion protein” is well known in the art, referring to a single polypeptide chain artificially designed which comprises two or more sequences from different origins, natural and/or artificial. The fusion protein, per definition, is never found in nature as such.

The term “single polypeptide chain”, as used herein means that the polypeptide components of the fusion protein can be conjugated end-to-end but also may include one or more optional peptide or polypeptide “linkers” or “spacers” intercalated between them, linked by a covalent bond.

In another embodiment, the polypeptide of interest is an antibody of a fragment of an antibody.

As used herein, the term “antibody” relates to a monomeric or multimeric protein which comprises at least one polypeptide having the capacity for binding to a determined antigen, or epitope within the antigen, and comprising all or part of the light or heavy

The term antibody also includes any type of known antibody, such as, for example, polyclonal antibodies, monoclonal antibodies and genetically engineered antibodies, such as chimeric antibodies, humanized antibodies, primatized antibodies, human antibodies, camelid antibodies and bispecific antibodies (including diabodies), multispecific antibodies (e.g. bispecific antibodies), and antibody fragments so long as they exhibit the desired biological activity.

The term “antibody fragment” includes antibody fragments such as Fab, F(ab′)2, Fab′, single chain Fv fragments (scFv), diabodies and nanobodies.

An illustrative non-limitative example of antibody is an antibody against the DEC-205 receptor. The term “DEC-205 receptor”, or “lymphocyte antigen 75”, or “C-type lectin domain family 13 member B”, as used herein, refers to a protein which acts as an endocytic receptor to direct captured antigens from the extracellular space to a specialized antigen-processing compartment and is found mainly on dendritic cells. In certain embodiments, the DEC-205 is the human protein defined by the UniProtKB/Swiss-Prot accession number 060449 (entry version 170 of 31 Jul. 2019, sequence version 3 of 11 Jan. 2011). In certain embodiments, the anti-DEC205 antibody is a monoclonal antibody. The anti-DEC-205 antibody can be of any origin, for example, from mouse, rabbit, human, or can be a humanized antibody. In certain embodiments, the compound of interest is a chain of the anti-DEC-205 antibody; in certain embodiments, the heavy chain. In another embodiment, the compound of interest is the heavy chain of the mouse αDEC-205 monoclonal antibody, as described by Stevens et al., JACS 2016, 138: 2162-5.

In another embodiment, the compound of interest is a fragment of a protein; in certain embodiments, a fragment of a protein of more than 25 KDa, more than 50 KDa or more than 100 KDa.

In another embodiment, the compound of interest is an N-terminal fragment of a protein; in certain embodiments, a fragment of a protein of more than 25 KDa, more than 50 KDa or more than 100 KDa. The term “N-terminal fragment of a protein”, as used herein, refers to a fragment of variable length that includes the N-terminus of the protein. In certain embodiments, the N-terminal fragment is a fragment comprising less than 100%, less than 90%, less than 80%, less than 70%, less than 60%, less than 50%, less than 40%, less than 30%, less than 20%, less than 10%, less than 5% of the length of the whole protein.

In certain embodiments, the complex comprises a split intein N-fragment comprising or consisting of an amino acid sequence selected from the group consisting of SEQ ID NO: 111, 112 and 113.

In certain embodiments, the sequences of SEQ ID NO: 112 and 113 have higher thermal stability than the sequence of SEQ ID NO: 1.

In certain embodiments, the complex comprises a split intein N-fragment comprising or consisting of an amino acid sequence selected from the group consisting of SEQ ID NO: 49-68 or variant thereof. In certain embodiments, the variant is a functionally equivalent variant.

The terms “variant” and “functionally equivalent variant” have been previously defined. In certain embodiments, the functionally equivalent variants of the split intein N-fragments of SEQ ID NO: 49-68 have at least 60%, at least 65%, at least 70%, at least 75%, at least 80%, at least 85%, at least 90%, at least 95% or at least 99% sequence identity with the sequence from which they derive.

In certain embodiments, the functionally equivalent variants of the split intein N-fragments of SEQ ID NO: 49-68 maintain or improve the activity from the sequence from which they derive. The term “activity” as well as methods to measure this activity have been previously defined in connection with the functionally equivalent variants of the split intein N-fragment of SEQ ID NO: 1. The embodiments regarding the activity of the variants of the split intein N-fragment of SEQ ID NO: 1 fully applies to the activity of the variants of the split intein N-fragments of SEQ ID NO: 49-68.

### Split Intein C-Fragment

In another aspect, this disclosure relates to a split intein C-fragment comprising the amino acid sequence of SEQ ID NO: 7 or a variant thereof having at least 88% sequence identity with SEQ ID NO: 7.

As interchangeably used herein, the terms “split intein C-fragment”, “C-terminal split intein”, “C-terminal intein fragment” and “C-terminal intein sequence” (abbreviated “Int^{C}”) refer to any intein sequence that comprises a C-terminal amino acid sequence that is functional for trans-splicing reactions, that is, that is capable of associating with a functional split intein N-fragment to form a complete intein that is capable of excising itself from the host protein, catalyzing the ligation of the extein or flanking sequences with a peptide bond, or that upon association with a split N-intein catalyzes the “C-terminal cleavage”, that is, the nucleophilic attack of the peptide bond between the extein and the C-terminus of the split intein C-fragment resulting in the breaking of said peptide bond. An Int^{C} thus also comprises a sequence that is spliced out when trans-splicing occurs. An Int^{C} can comprise a sequence that is a modification of the C-terminal portion of a naturally occurring intein sequence. For example, it can comprise additional amino acid residues and/or mutated residues so long as the inclusion of such additional and/or mutated residues does not render the Int^{C} non-functional in trans-splicing. In certain embodiments, the inclusion of the additional and/or mutated residues improves or enhances the trans-splicing activity of the Inti.

In certain embodiments, the split intein C-fragment comprises the amino acid sequence of SEQ ID NO: 7. The split intein C-fragment can comprise additional amino acid residues linked to the N- and/or C-terminus of the sequence of SEQ ID NO: 7. In certain embodiments, the split intein C-fragment comprises less than 10, less than 9, less than 8, less than 7, less than 6, less than 5, less than 4, less than 3, less than 2, or 1 additional amino acid residues linked to the N- and/or C-terminus of the sequence of SEQ ID NO: 7. In another embodiment, the split intein N-fragment consists on the amino acid sequence of SEQ ID NO: 7.

In certain embodiments, the split intein C-fragment comprises or consists on a variant of the amino acid sequence of SEQ ID NO: 7 having at least 88% sequence identity with SEQ ID NO: 7.

The terms “amino acid” and “variant” have been already described within the context of the N-inteins and equally apply to the present case.

The variant of the split intein C-fragment of SEQ ID NO: 7 has at least 88% sequence identity with SEQ ID NO: 7. In certain embodiments, the variant of the split intein C-fragment of SEQ ID NO: 7 has at least 89%, at least 90%, at least 91%, at least 92%, at least 93%, at least 94%, at least 95%, at least 96%, at least 97%, at least 98%, at least 99% sequence identity with SEQ ID NO: 7.

In certain embodiments, the variant of the split intein C-fragment of SEQ ID NO: 7 has a length of between 50 and 160 amino acids; and in certain embodiments, 60, 65, 70, 75, 80, 85, 90, 95, 100, 105, 110, 115, 120, 125, 130, 135, 140, 145, 150, 155 or 160 amino acids.

In certain embodiments, the variant of the split intein C-fragment of SEQ ID NO: 7 has at least 88% sequence identity with SEQ ID NO: 7 over the whole length of the sequence.

In certain embodiments, the variant of the split intein C-fragment of sequence SEQ ID NO: 7 comprises or consist on an amino acid sequence selected from the group consisting of SEQ ID NO: 848 and 128-166.

In another embodiment, the variant of the split C-intein of SEQ ID NO: 7 is a functionally equivalent variant of SEQ ID NO: 7.

The term “functionally equivalent variant” has been previously defined for the split intein C-fragment. In the case of the functionally equivalent variant of the split intein C-fragment of SEQ ID NO: 7, the activity of the split intein C-fragment refers to its ability to bind to a split intein N-fragment and catalyze the “C-terminal cleavage”, that is, the nucleophilic attack of the peptide bond between the extein and the C-terminus of the split intein C-fragment, resulting in the breaking of said peptide bond. The activity of the split intein C-fragment can also refer to the “trans-splicing activity”, which is understood as the ability of said split intein C-fragment to bind to a functional split intein N-fragment excising the complete intein from the host protein, catalyzing the ligation of the extein or flanking sequences with a peptide bond. Suitable assays for determining whether a polypeptide is a functionally equivalent variant of a given split C-intein, in terms of its trans-splicing activity, include splicing assays, such as those describe in example the methods of the present application or disclosed in Shah N H et al (Shah N T et al., 2012, J Chem Soc, vol 134, 11338), as long as in these assays the split intein C-fragment is combined with a functional split intein N-fragment, that is a split intein N-fragment which is capable of catalyzing the N-terminal cleavage. Other more specific assays have also been described which allow characterizing each of the steps of the protein splicing, and particularly the last step involving the cleavage of the peptide bond between the C-intein and the C-extein, herein referred as “C-terminal cleavage” (Shah et al. JACS 2013).

According to the present disclosure, the activity of an C-intein is substantially maintained if the functionally equivalent has at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the activity of the intein of the claimed sequences. Furthermore, the activity of the C-intein is substantially improved if the functionally equivalent variant has at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, or at least 65%, at least 70%, at least 75%, at least 80%, at least 85%, at least 90%, at least 95%, at least 100%, at least 150%, at least 200%, at least 300%, at least 400%, at least 500%, at least 1000%, or more of the activity of the C-inteins of this disclosure.

As mentioned above, the activity of the split intein C-fragment of this disclosure depend on a number of reaction parameters, including temperature, chaotropic environment and pH. Thus, in one embodiment, the functionally equivalent variant of the split intein C-fragment of this disclosure maintains or improve its activity at a temperature of at least 0° C., at least 5° C., at least 10° C., at least 15° C., at least 20° C., at least 25° C., at least 30° C., at least 35° C., at least 37° C., at least 40° C., at least 45° C., at least 50° C., at least 55° C., at least 60° C., at least 65° C., at least 70° C. or higher. In certain embodiments, the functionally equivalent variant of the split intein C-fragment of this disclosure maintains or improve its activity at a temperature of 50° C. Likewise, in another embodiment the functionally equivalent variant of the split intein C-fragment of this disclosure maintains or improves its activity at least at pH 0.1, or at least at pH 0.5, or at least at pH 1.0, or at least at pH 1.5, or at least at pH 2.0, or at least at pH 2.5, or at least at pH 3.0, or at least at pH 3.5, or at least at pH 4.0, or at least at pH 4.5, or at least at pH 5.0, or at least at pH 5.5, or at least at pH 6.0, or at least at pH 6.5, or at least at pH 7.0, or at least at pH 7.2, or at least at pH 7.5, or at least at pH 8.0, or at least at pH 8.5, or at least at pH 9.0, or at least at pH 9.5, or at least at pH 10.0, or at least at pH 10.5, or at least at pH 1 1.0, or at least at pH 11.5, or at least at pH 12.0, or at least at pH 12.5, or at least at pH 13.0, or at least at pH 13.5, or at pH 14. In certain embodiments, the functionally equivalent variant of the split intein C-fragment of this disclosure maintains or improves its activity at pH 7.2. In another embodiment, the functionally equivalent variant of the split intein C-fragment of this disclosure maintains or improves its activity at urea 1 M, or at least at urea 1.5 M, or at least urea 2 M, or at least urea 3 M, or at least urea 3.5 M, or at least urea 4 M, or at least urea 4.5 M, or at least urea 5 M. In certain embodiments, the functionally equivalent variant of the split C-intein of this disclosure maintains or improves its activity at urea 2 M or urea 5 M. In certain embodiments, the functionally equivalent variant of the split C-intein of this disclosure maintains or improves its activity at a temperature of 50° C., at pH 7.2 and at urea 2 M or urea 4 M. All possible combinations of temperatures, urea concentration and pH are also contemplated by this disclosure.

In certain embodiments, the functionally equivalent variant of the split intein C-fragment of this disclosure that maintains or improves its activity has at least 88%, at least 89%, at least 90%, at least 91%, at least 92%, at least 93%, at least 94%, at least 95%, at least 96%, at least 97%, at least 98% or at least 99% sequence identity with SEQ ID NO: 7.

In another embodiment, the functionally equivalent variant of the split intein C-fragment comprises or consist on an amino acid sequence selected from the group consisting of SEQ ID NO: 10-22 and 128-140.

### Complex Comprising a Split Intein C-Fragment

In another aspect, this disclosure relates to a complex, hereinafter second complex of this disclosure, comprising:
(i) the split intein C-fragment of SEQ ID NO: 7 or a split intein C-fragment comprising a sequence selected from the group consisting of SEQ ID NO: 114-120 and
(ii) a compound of interest
   wherein the complex optionally comprises a linker between (i) and (ii) and
   wherein
the compound of interest is bound to the C-terminus of the split intein C-fragment by an amide linkage or
if the complex comprises a linker, the compound of interest if bound to the linker by an amide linkage and/or the linker is bound to the C-terminus of the split intein C-fragment by and amide linkage.

The terms “compound of interest” and “linker” have been previously defined in connection with the first complex of this disclosure. All the embodiments of the compound of interest and linker of the first complex of this disclosure fully apply to the second complex of this disclosure.

In certain embodiments, the complex does not comprise a linker between the compound of interest and the split intein C-fragment. In this embodiment, the compound of interest is linked to the C-terminus of the split intein C-fragment by an amide linkage.

In certain embodiments, the complex comprises a linker between the compound of interest and the split intein C-fragment. In this embodiment, the compound of interest may be bound to the linker by any suitable means, depending on the chemical nature of the compound of interest and of the linker. In this embodiment, the linker is bound to the C-terminus of the split intein C-fragment by an amide linkage. In another embodiment, the compound of interest is bound to the linker by an amide linkage, in which case the linker may be bound to the C-terminus of the split intein C-fragment by any suitable means. In another embodiment, the compound of interest is bound to the linker by an amide linkage and the linker is bound to the C-terminus of the split intein C-fragment by an amide linkage.

In another embodiment, the compound of interest is a protein having the N-terminal amino acid residues of the extein capable of being spliced by an intein comprising the split intein C-fragment of sequence SEQ ID NO: 7. In another embodiment, the compound of interest is a protein having the sequence Cys-Xaa₁-Xaa₂ or Cys-Xaa₁-Xaa₂-Leu in its N-terminus, where:
Xaa₁ and Xaa₂ are any amino acid;
Xaa₁ is Ala, Gly, Art or Phe and Xaa₂ is any amino acid;
Xaa₁ is any amino acid and Xaa₂ is Gly, Glu, Ala or Arg;
Xaa₁ is Ala, Gly, Art or Phe and Xaa₂ is Gly, Glu, Ala or Arg.

In another embodiment, the compound of interest is a protein having a sequence selected from Cys-Glu-Phe, Cys-Ala-Phe; Cys-Gly-Phe; Cys-Arg-Phe, Cys-Phe-Phe, Cys-Glu-Gly, Cys-Glu-Glu, Cys-Glu-Ala, Cys-Glu-Phe-Leu, Cys-Ala-Phe-Leu; Cys-Gly-Phe-Leu; Cys-Arg-Phe-Leu, Cys-Phe-Phe-Leu, Cys-Glu-Gly-Leu, Cys-Glu-Glu-Leu and Cys-Glu-Ala-Leu in its N-terminus.

In another embodiment, when the compound of interest is not a protein, the C-intein comprises or consists on a polypeptide selected from the group consisting of SEQ ID NO: 10-48 or SEQ ID NO: 128-166. In another embodiment, when the compound of interest is not a protein, the compound of interest and the C-intein are joined through a linker in which case, the linker is a peptide having the N-terminal amino acid residues of the extein capable of being spliced by an intein comprising the split intein C-fragment of sequence SEQ ID NO: 7; in certain embodiments, the linker is a peptide having the sequence Cys-Xaa₁-Xaa₂ or Cys-Xaa₁-Xaa₂-Leu in its N-terminus, where:
Xaa₁ and Xaa₂ are any amino acid;
Xaa₁ is Ala, Gly, Art or Phe and Xaa₂ is any amino acid;
Xaa₁ is any amino acid and Xaa₂ is Gly, Glu, Ala or Arg;
Xaa₁ is Ala, Gly, Art or Phe and Xaa₂ is Gly, Glu, Ala or Arg;
   or the linker is a peptide having a sequence selected from Cys-Glu-Phe, Cys-Ala-Phe, Cys-Gly-Phe, Cys-Arg-Phe, Cys-Phe-Phe, Cys-Glu-Gly, Cys-Glu-Glu, Cys-Glu-Ala, Cys-Glu-Phe-Leu, Cys-Ala-Phe-Leu, Cys-Gly-Phe-Leu, Cys-Arg-Phe-Leu, Cys-Phe-Phe-Leu, Cys-Glu-Gly-Leu, Cys-Glu-Glu-Leu and Cys-Glu-Ala-Leu in its N-terminus.

In another embodiment, the compound of interest is a protein that does not have the N-terminal amino acid residues of the extein capable of being spliced by an intein comprising the split C-intein of SEQ ID NO: 7, in which case (i) the C-intein comprises or consists on the polypeptide of sequence SEQ ID NO: 10-44 or 128-166 or (ii) the compound of interest and the C-intein are joined through a linker in which case, the linker is a peptide having the C-terminal amino acid residues of the extein capable of being spliced by an intein comprising the split intein C-fragment of SEQ ID NO: 7; in certain embodiments, the linker is a peptide having the sequence Cys-Xaa₁-Xaa₂ or Cys-Xaa₁-Xaa₂-Leu in its N-terminus, where:
Xaa₁ and Xaa₂ are any amino acid;
Xaa₁ is Ala, Gly, Art or Phe and Xaa₂ is any amino acid;
Xaa₁ is any amino acid and Xaa₂ is Gly, Glu, Ala or Arg;
Xaa₁ is Ala, Gly, Art or Phe and Xaa₂ is Gly, Glu, Ala or Arg;
   or the linker is a peptide having a sequence selected from Cys-Glu-Phe, Cys-Ala-Phe, Cys-Gly-Phe, Cys-Arg-Phe, Cys-Phe-Phe, Cys-Glu-Gly, Cys-Glu-Glu, Cys-Glu-Ala, Cys-Glu-Phe-Leu, Cys-Ala-Phe-Leu; Cys-Gly-Phe-Leu; Cys-Arg-Phe-Leu, Cys-Phe-Phe-Leu, Cys-Glu-Gly-Leu, Cys-Glu-Glu-Leu and Cys-Glu-Ala-Leu in its N-terminus.

In certain embodiments, the compound of interest is a protein or polypeptide.

In another embodiment, the compound of interest is a protein of more than 25 KDa, more than 50 KDa or more than 100 KDa.

In certain embodiments, the protein is Cas9 or a fragment of Cas9. In certain embodiments, the compound of interest is a polypeptide or protein, and if the complex comprises a linker, the linker is a peptide linker. In this embodiment, the complex is a fusion protein.

In another embodiment, the polypeptide of interest is an antibody or a fragment of an antibody. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDec205 monoclonal antibody, as described by Stevens et al., JACS 2016, 138: 2162-5.

In another embodiment, the compound of interest is a fragment of a protein; in certain embodiments, a fragment of a protein of more than 25 KDa, more than 50 KDa or more than 100 KDa. In another embodiment, the compound of interest is a C-terminal fragment of a protein. The term “C-terminal fragment of a protein”, as used herein, refers to a fragment of variable length that includes the C-terminus of the protein. In certain embodiments, the C-terminal fragment is a fragment comprising less than 100%, less than 90%, less than 80%, less than 70%, less than 60%, less than 50%, less than 40%, less than 30%, less than 20%, less than 10%, less than 5% of the length of the whole protein.

In another embodiment, the compound of interest is an antibody. The term antibody has been described within the context of the N-inteins and equally apply to the present case.

In certain embodiments, the complex comprises a split intein C-fragment comprising or consisting of an amino acid sequence selected from the group consisting of SEQ ID NO: 114-120.

In certain embodiments, the sequences of SEQ ID NO: 123 and 124 have higher thermal stability than the sequence of SEQ ID NO: 7.

In certain embodiments, the complex comprises a split intein C-fragment comprising or consisting of an amino acid sequence selected from the group consisting of SEQ ID NO: 69-87 or a variant thereof. In certain embodiments, the variant is a functionally equivalent variant.

The terms “variant” and “functionally equivalent variant” have been previously defined. In certain embodiments, the functionally equivalent variants of the split intein C-fragments of SEQ ID NO: 69-87 have at least 60%, at least 65%, at least 70%, at least 75%, at least 80%, at least 85%, at least 90%, at least 95% or at least 99% sequence identity with the sequence from which they derive.

In certain embodiments, the functionally equivalent variants of the split intein C-fragments of SEQ ID NO: 69-87 maintain or improve the activity from the sequence from which they derive. The term “activity” as well as methods to measure this activity have been previously defined in connection with the functionally equivalent variants of the split intein N-fragment of SEQ ID NO: 7. The embodiments regarding the activity of the variants of the split intein C-fragment of SEQ ID NO: 7 fully applies to the activity of the variants of the split intein C-fragments of SEQ ID NO: 69-87.

### Complex Comprising a Split Intein N-Fragment and a Split Intein C-Fragment

In another aspect, this disclosure relates to a complex, hereinafter third complex of this disclosure, comprising:
(iv) the split intein C-fragment of this disclosure or a split intein C-fragment comprising a sequence selected from the group consisting of SEQ ID NO: 114-120
(v) a compound of interest and
(vi) the split intein N-fragment of this disclosure, or a split intein N-fragment comprising the amino acid sequence selected from the group consisting of SEQ ID NO: 103-110
   wherein the complex optionally comprises a linker between (i) and (ii) and/or between (ii) and (iii),
   wherein
the compound of interest is linked to the C-terminus of the split intein C-fragment by an amide linkage or
if the complex comprises a linker, the compound of interest is bound to the linker by an amide linkage and/or the linker is bound to the C-terminus of the split intein C-fragment by an amide linkage and
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.

The terms “compound of interest” and “linker” have been previously defined in connection with the first complex of this disclosure. All the embodiments of the compound of interest and linker of the first complex of this disclosure fully apply to the second complex of this disclosure.

In certain embodiments, the compound of interest is a protein or polypeptide.

In another embodiment, the compound of interest is a protein of more than 25 KDa, more than 50 KDa or more than 100 KDa. In certain embodiments, the compound of interest is a polypeptide or protein, and if the complex comprises a linker, the linker is a peptide linker. In this embodiment, the complex is a fusion protein.

In certain embodiments, the polypeptide of interest is an antibody of a fragment of an antibody. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDEC-205 monoclonal antibody, as described by Stevens et al., JACS 2016, 138: 2162-5.

In certain embodiments, the complex comprises a split intein C-fragment comprising or consisting of an amino acid sequence selected from the group consisting of SEQ ID NO: 114-120.

In certain embodiments, the sequences of SEQ ID NO: 123 and 124 have higher thermal stability than the sequence of SEQ ID NO: 7.

In certain embodiments, the complex comprises a split intein C-fragment comprising or consisting of an amino acid sequence selected from the group consisting of SEQ ID NO: 69-87 or a variant thereof. In certain embodiments, the variant is a functionally equivalent variant.

In certain embodiments, the complex comprises a split intein N-fragment comprising or consisting of an amino acid sequence selected from the group consisting of SEQ ID NO: 111, 112 and 113.

In certain embodiments, the sequences of SEQ ID NO: 112 and 113 have higher thermal stability than the sequence of SEQ ID NO: 1.

In certain embodiments, the complex comprises a split intein N-fragment comprising or consisting of an amino acid sequence selected from the group consisting of SEQ ID NO: 49-68 or a variant thereof. In another embodiment, the variant is a functionally equivalent variant.

The terms “variant” and “functionally equivalent variant” have been previously defined. The embodiments regarding these terms fully applies to the third complex of this disclosure.

Composition Comprising the Complexes of this Disclosure

In another aspect, this disclosure relates to a composition, hereinafter first composition of this disclosure, comprising the first and the second complex of this disclosure.

The term “composition” is intended to encompass a product containing the specified components, as well as any product that results, directly or indirectly, from a combination of the specified components in the specified amounts. The components of the composition may be packed together in a single formulation or separately in different formulations. Thus in an embodiment the first complex of this disclosure is packed together with the second complex of this disclosure in a single formulation. In another embodiment, the first complex of this disclosure and of the second complex of this disclosure are separately packed.

In one embodiment, the first and the second complex comprise the N-terminal fragment and the C-terminal fragment of the same protein respectively, in such a way that when both complexes are combined according to the methods of this disclosure, the N-terminal fragment of the protein is linked to the C-terminal fragment of the protein generating the whole protein.

Conjugates of this Disclosure

In another aspect, this disclosure relates to a conjugate, hereinafter first conjugate of this disclosure, comprising the first complex of this disclosure and the second complex of this disclosure, wherein the C-terminus of the split intein N-fragment is linked to the N-terminus of the split intein C-fragment by a peptide bond.

In another aspect, this disclosure relates to a conjugate, hereinafter second conjugate of this disclosure, comprising (a) the first complex of this disclosure and (b) a split intein C-fragment comprising the amino acid sequence of SEQ ID NO: 7 or a variant thereof having at least 88% sequence identity with SEQ ID NO: 7 or an amino acid sequence selected from the group consisting of SEQ ID NO: 114-120, wherein the C-terminus of the split intein N-fragment is linked to the N-terminus of the split intein C-fragment by a peptide bond.

In certain embodiments, the conjugate comprises a split intein C-fragment comprising or consisting of a sequence selected from SEQ ID NO: 121-124.

In certain embodiments, the conjugate comprises a split intein C-fragment comprising or consisting of a sequence selected from SEQ ID NO: 69-87 or a variant thereof. In certain embodiments, the variant is a functionally equivalent variant. The functionally equivalent variants of the split intein C-fragment of SEQ ID NO: 69-87 have been previously defined.

In certain embodiments, the compound of interest is a protein or polypeptide.

In another embodiment, the compound of interest is a protein of more than 25 KDa, more than 50 KDa or more than 100 KDa.

In certain embodiments, the protein is Cas9 or a fragment of Cas9.

In certain embodiments, the compound of interest is a polypeptide or protein, and if the complex comprises a linker, the linker is a peptide linker.

In certain embodiments, the polypeptide of interest is an antibody or a fragment of an antibody. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDEC-205 monoclonal antibody, as described by Stevens et al., JACS 2016, 138: 2162-5.

Polynucleotides, Vectors and Host Cells of this Disclosure

In another aspect, this disclosure relates to a polynucleotide encoding:
the split intein N-fragment of this disclosure, or
the split intein C-fragment of this disclosure, or
the first, second or third complex of this disclosure, wherein the compound of interest is a polypeptide or protein and the linker, if present, is a peptide linker, or
the conjugate of this disclosure.

As used herein, the term “polynucleotide” refers to a polymer composed of a multiplicity of nucleotide units (deoxyribonucleotides or ribonucleotides, or related structural variants or synthetic analogues thereof) linked via phosphodiester bonds (or related structural variants on synthetic analogues thereof). The term polynucleotide includes double or single stranded genomic and cDNA, RNA, any synthetic and genetically manipulated polynucleotide, and both sense and anti-sense polynucleotide (although only sense stands are being disclosed in the present disclosure). This includes single- and double-stranded molecules, i.e., DNA-DNA, DNA-RNA and RNA-RNA hybrids.

The polynucleotide of this disclosure can be found isolated as such or forming part of vectors allowing the propagation of said polynucleotides in suitable host cells. Therefore, in another aspect, this disclosure relates to a vector comprising the polynucleotide of this disclosure as described above.

Vectors suitable for the insertion of said polynucleotide are vectors derived from expression vectors in prokaryotes such as pUC18, pUC19, Bluescript and the derivatives thereof, mpI8, mpI9, pBR322, pMB9, ColEI, pCRI, RP4, phages and “shuttle” vectors such as pSA3 and pAT28; expression vectors in yeasts such as vectors of the type of 2 micron plasmids, integration plasmids, YEP vectors, centromere plasmids and the like; expression vectors in insect cells such as vectors of the pAC series and of the pVL; expression vectors in plants such as pIBI, pEarleyGate, pAVA, pCAMBIA, pGSA, pGWB, pMDC, pMY, pORE series and the like; and expression vectors in eukaryotic cells, including baculovirus suitable for transfecting insect cells using any commercially available baculovirus system. The vectors for eukaryotic cells include viral vectors (adenoviruses, adeno associated viruses (AAV),retroviruses and lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6/V5-His, pVAXI, pZeoSV2, pCI, pSVL and PKSV-10, pBPV-1, pML2d and pTDTI.

The vectors may also comprise a reporter or marker gene which allows identifying those cells that have incorporated the vector after having been put in contact with it.

Useful reporter genes in the context of the present disclosure include lacZ, luciferase, thymidine kinase, GFP and on the like. Useful marker genes in the context of this disclosure include, for example, the neomycin resistance gene, conferring resistance to the aminoglycoside G418; the hygromycin phosphotransferase gene, conferring resistance to hygromycin; the ODC gene, conferring resistance to the inhibitor of the ornithine decarboxylase (2-(difluoromethyl)-DL-ornithine (DFMO); the dihydrofolatereductase gene, conferring resistance to methotrexate; the puromycin-N-acetyl transferase gene, conferring resistance to puromycin; the ble gene, conferring resistance to zeocin; the adenosine deaminase gene, conferring resistance to 9-beta-D-xylofuranose adenine; the cytosine deaminase gene, allowing the cells to grow in the presence of N-(phosphonacetyl)-L-aspartate; thymidine kinase, allowing the cells to grow in the presence of aminopterin; the xanthine-guanine phosphoribosyltransferase gene, allowing the cells to grow in the presence of xanthine and the absence of guanine; the trpB gene of E. coli, allowing the cells to grow in the presence of indol instead of tryptophan; the hisD gene of E. coli, allowing the cells to use histidinol instead of histidine. The selection gene is incorporated into a plasmid that can additionally include a promoter suitable for the expression of said gene in eukaryotic cells (for example, the CMV or SV40 promoters), an optimized translation initiation site (for example, a site following the so-called Kozak's rules or an IRES), a polyadenylation site such as, for example, the SV40 polyadenylation or phosphoglycerate kinase site, introns such as, for example, the beta-globulin gene intron. Alternatively, it is possible to use a combination of both the reporter gene and the marker gene simultaneously in the same vector.

On the other hand, as the skilled person in the art knows, the choice of the vector will depend on the host cell in which it will subsequently be introduced. By way of example, the vector in which said polynucleotide is introduced can also be a yeast artificial chromosome (YAC), a bacterial artificial chromosome (BAC) or a PI-derived artificial chromosome (PAC). The characteristics of the YAC, BAC and PAC are known by the person skilled in the art. Detailed information on said types of vectors has been provided, for example, by Giraldo and Montoliu (Giraldo, P. & Montoliu L., 2001 Size matters: use of YACs, BACs and PACs in transgenic animals, Transgenic Research 10(2): 83-110). The vector of this disclosure can be obtained by conventional methods known by persons skilled in the art (Sambrook J. et al., 2000 “Molecular cloning, a Laboratory Manual”, 3rd ed., Cold Spring Harbor Laboratory Press, N.Y. Vol 1-3).

The polynucleotide of this disclosure can be introduced into the host cell in vivo as naked DNA plasmids, but also using vectors by methods known in the art, including but not limited to transfection, electroporation (e.g. transcutaneous electroporation), microinjection, transduction, cell fusion, DEAE dextran, calcium phosphate precipitation, use of a gene gun, or use of a DNA vector transporter. Methods for formulating and administering naked DNA to mammalian muscle tissue are also known. See Feigner P, et al., U.S. Pat. Nos. 5,580,859, and 5,589,466. Other molecules are also useful for facilitating transfection of a nucleic acid in vivo, such as cationic oligopeptides, peptides derived from DNA binding proteins, or cationic polymers. See Bazile D, et al., WO 1995021931, and Byk G, et al., WO 1996025508.

Another well-known method that can be used to introduce polynucleotides into host cells is particle bombardment (aka biolistic transformation). Biolistic transformation is commonly accomplished in one of several ways. One common method involves propelling inert or biologically active particles at cells. See Sanford J, et al., U.S. Pat. Nos. 4,945,050, 5,036,006, and 5,100,792.

Alternatively, the vector can be introduced in vivo by lipofection. The use of cationic lipids can promote encapsulation of negatively charged nucleic acids, and also promote fusion with negatively charged cell membranes. See Feigner P, Ringold G, Science 1989; 337:387-388. Useful lipid compounds and compositions for transfer of nucleic acids have been described. See Feigner P, et al., U.S. Pat. No. 5,459,127, Behr J, et al., WO1995018863, and Byk G, WO1996017823.

Thus, in another aspect, this disclosure relates to a host cell comprising the polynucleotide or the vector of this disclosure. The cells can be obtained by conventional methods known by persons skilled in the art (see e.g. Sambrook et al., cited ad supra).

The term “host cell”, as used herein, refers to a cell into which a nucleic acid of this disclosure, such as a polynucleotide or a vector according to this disclosure, has been introduced and is capable of expressing the split intein N-fragment of this disclosure or the fusion protein comprising said split intein N-fragment. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact be identical to the parent cell, but are still included within the scope of the term as used herein. The term includes any cultivatable cell that can be modified by the introduction of heterologous DNA. In certain embodiments, a host cell is one in which the polynucleotide of this disclosure can be stably expressed, post-translationally modified, localized to the appropriate subcellular compartment, and made to engage the appropriate transcription machinery. The choice of an appropriate host cell will also be influenced by the choice of detection signal. For example, reporter constructs, as described above, can provide a selectable or screenable trait upon activation or inhibition of gene transcription in response to a transcriptional regulatory protein; in order to achieve optimal selection or screening, the host cell phenotype will be considered. A host cell of the present disclosure includes prokaryotic cells and eukaryotic cells. Prokaryotes include gram negative or gram positive organisms, for example, E. coli or Bacilli. It is to be understood that in certain embodiments prokaryotic cells will be used for the propagation of the transcription control sequence comprising polynucleotides or the vector of the present disclosure. Suitable prokaryotic host cells for transformation include, for example, E. coli, Bacillus subtilis, Salmonella typhimurium, and various other species within the genera Pseudomonas, Streptomyces, and Staphylococcus. Eukaryotic cells include, but are not limited to, yeast cells, plant cells, fungal cells, insect cells (e.g., baculovirus), mammalian cells, and the cells of parasitic organisms, e.g., trypanosomes. As used herein, yeast includes not only yeast in a strict taxonomic sense, i.e., unicellular organisms, but also yeast-like multicellular fungi of filamentous fungi. Exemplary species include Kluyverei lactis, Schizosaccharomyces pombe, and Ustilaqo maydis, and Saccharomyces cerevisiae. Other yeasts which can be used in practicing the present disclosure are Neurospora crassa, Aspergillus niger, Aspergillus nidulans, Pichia pastoris, Candida tropicalis, and Hansenula polymorpha. Mammalian host cell culture systems include established cell lines such as COS cells, L cells, 3T3 cells, Chinese hamster ovary (CHO) cells, embryonic stem cells, BHK, HeK, or HeLa cells. In certain embodiments, eukaryotic cells are used for recombinant gene expression.

### Methods to Conjugate Two Compounds of Interest

In another aspect, this disclosure relates to a method to obtain a conjugate between a first compound of interest and a second compound of interest comprising:

(i) contacting
(a) the first complex of this disclosure, wherein the complex comprises the first compound of interest and a split intein N-fragment comprising the amino sequence of SEQ ID NO: 1 or a functionally equivalent variant thereof having at least 90% sequence identity with SEQ ID NO: 1, or an amino acid sequence selected from the group consisting of SEQ ID NO: 103-110 with
(b) the second complex of this disclosure, wherein the complex comprises the second compound of interest and a split intein C-fragment comprising the amino acid sequence of SEQ ID NO: 7 or a functionally equivalent variant thereof having at least 88% sequence identity with SEQ ID NO: 7, or an amino acid sequence selected from the group consisting of SEQ ID NO: 114-120
or
a complex comprising an AceL-TerL split intein C-fragment or a functionally equivalent variant thereof and the second compound of interest, wherein the complex optionally comprises a linker between the split intein C-fragment and the second compound of interest and wherein
   the second compound of interest is bound to the C-terminus of the split intein C-fragment by an amide linkage or
   if the complex comprises a linker, the second compound of interest is bound to the linker by an amide linkage and/or the linker is bound to the C-terminus of the split intein C-fragment by an amide linkage
   under appropriate conditions for binding the split intein N-fragment to the split intein C-fragment to form an intein intermediate and
      (ii) allowing the intein intermediate to react to form a conjugate between the first and the second compound of interest.

In another aspect, this disclosure relates to a method to obtain a conjugate between a first compound of interest and a second compound of interest comprising

(i) contacting
(a) the first complex of this disclosure, wherein the complex comprises the first compound of interest and a split intein N-fragment comprising the amino sequence of SEQ ID NO: 1 or a functionally equivalent variant thereof having at least 90% sequence identity with SEQ ID NO: 1, or an amino acid sequence selected from the group consisting of SEQ ID NO: 103-110
   or
   a complex comprising complex comprising a compound of interest and an AceL-TerL split intein N-fragment or a functionally equivalent variant thereof, wherein the complex optionally comprises a linker between the compound of interest and the split intein N-fragment, and wherein
      the compound of interest is linked to the N-terminus of the split intein N-fragment by an amide linkage or
      if the complex comprises a linker, the compound of interest is bound to the linker by an amide linkage and/or the linker is bound to the N-terminus of the split intein N-fragment by an amide linkage.
         with
(b) the complex of any one of claims 17 to 21, wherein the complex comprises the second compound of interest and a split intein C-fragment comprising the amino acid sequence of SEQ ID NO: 7 or a functionally equivalent variant thereof having at least 88% sequence identity with SEQ ID NO: 7, or an amino acid sequence selected from the group consisting of SEQ ID NO: 114-120
   under appropriate conditions for binding the split intein N-fragment to the split intein C-fragment to form an intein intermediate and
(ii) allowing the intein intermediate to react to form a conjugate between the first and the second compound of interest.

The term “AceL-TerL intein”, as used herein, refers to a family of non-canonical split inteins identified in the Antarctic permanently stratified saline lake, Ace Lake. This family of inteins was described by Thiel et al., Angew. Chem. Int. Ed 2014, 53: 1306-1310. In certain embodiments, the AceL-TerL split intein N-fragment comprises or consists on the sequence of SEQ ID NO: 101 or 102. In certain embodiments, the AceL-TerL split intein C-fragment comprises or consists on the sequence of SEQ ID NO: 99 or 100.

The terms “compound of interest” and “functionally equivalent variant” have been previously defined. In some embodiments, the first compound and/or the second compound is or includes a peptide or a polypeptide. In some embodiments the first compound and/or the second compound is or includes an antibody, antibody chain, or antibody heavy chain. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDEC-205 monoclonal antibody, as described by Stevens et al., JACS 2016, 138: 2162-5.

In some embodiments, the first compound and/or the second compound is or includes a peptide, oligonucleotide, drug, or cytotoxic molecule.

In certain embodiments, the split intein N-fragment comprises or consists of a sequence selected from the group consisting of SEQ ID NO: 111-113.

In certain embodiments, the split intein N-fragment comprises or consists of a sequence selected from the group consisting of SEQ ID NO: 49-68 or a functionally equivalent variant thereof.

In certain embodiments, the split intein C-fragment comprises or consists of a sequence selected from the group consisting of SEQ ID NO: 121-124. In certain embodiments, the split intein C-fragment comprises or consists of a sequence selected from the group consisting of SEQ ID NO: 69-87 or a functionally equivalent variant thereof.

In certain embodiments, the split intein N-fragment comprises or consists of a sequence selected from the group consisting of SEQ ID NO: 49-68 or a functionally equivalent variant thereof and the split intein C-fragment comprises or consists of a sequence selected from the group consisting of SEQ ID NO: 69-87 or a functionally equivalent variant thereof.

The appropriate conditions for binding the split intein N-fragment to the split intein C-fragment to form an intein intermediate can be easily determined by the skilled person. In certain embodiments, these conditions involve contacting the first and second complex at temperature between 0° C. and 70° C., for example, between 5° C. and 65° C., between 10° C. and 60° C., between 15° C. and 55° C., between 20° C. and 50° C., between 25° C. and 45° C., between 30° C. and 40° C., between 25° C. and 35° C., between 45° C. and 55° C.; in certain embodiments at 30° C. or 50° C. In another embodiment the conditions involve contacting the first and second complex at a pH between 0.1 and 14, for example between 0.5 and 13.5, between 1.0 and 13.0, between 1.5 and 12.5, between 2.0 and 12.0, between 2.5 and 11.5, between 3.0 and 11.0, between 3.5 and 10.5, between 4.0 and 10.0, between 4.5 and 9.5, between 5.0 and 9.0, between 5.5 and 8.5, between 6.0 and 8.0, between 6.5 and 7.5; in certain embodiments at pH 7.2. In another embodiment, these conditions involve contacting the first and second complex in the absence of urea, or in the presence of urea at a concentration between 1 M and 5 M, for example between 1.5 M and 4.5 M, between 2 M and 4.0 M, between 2.5 M and 3.5 M; in certain embodiments at urea 2 M or at urea 4 M. In certain embodiments. In certain embodiments, these conditions involve contacting the first and second complex at a temperature of 50° C., at pH 7.2 and in the presence of urea 2 M or urea 4 M. All possible combinations of temperatures, urea concentration and pH are also contemplated by this disclosure.

### Method to Obtain a Conjugate of a Compound of Interest and a Nucleophile

In another aspect this disclosure relates to a method to obtain a conjugate of a compound of interest with a nucleophile comprising

(i) contacting
(a) the first complex of this disclosure, wherein the split intein N-fragment comprises the amino acid sequence of SEQ ID NO: 1 or a functionally equivalent variant thereof having at least 90% sequence identity with SEQ ID NO: 1 or an amino acid sequence selected from the group consisting of SEQ ID NO: 103-110,
or
a complex comprising a compound of interest and an AceL-TerL split intein N-fragment or a functionally equivalent variant thereof, wherein the complex optionally comprises a linker between the compound of interest and the split intein N-fragment, and wherein
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.
   with
   (b) a split intein C-fragment comprising an amino acid sequence selected from the group consisting of SEQ ID NO: 8, 9, 23-48 and 141-166, under appropriate conditions for binding between the split intein N-fragment and the split intein C-fragment to form an intein intermediate and
   (ii) contacting the intein intermediate with an exogenous nucleophile.

The terms “AceL-TerL split intein N-fragment”, “compound of interest” and “functionally equivalent variant” have been previously defined. In certain embodiments, the AceL-TerL split intein N-fragment comprises or consist on the sequence of SEQ ID NO: 101 or 102. In some embodiments, the first compound and/or the second compound is or includes a peptide or a polypeptide. In some embodiments the first compound and/or the second compound is or includes an antibody, antibody chain, or antibody heavy chain. In certain embodiments, the polypeptide of interest is an antibody or a fragment of an antibody. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDEC-205 monoclonal antibody, as described by Stevens et al., JACS 2016, 138: 2162-5.

In some embodiments, the first compound and/or the second compound is or includes a peptide, oligonucleotide, drug, or cytotoxic molecule.

The term “nucleophile,” as used herein, refers to any chemical species that donates an electron pair to an electrophile to form a chemical bond in relation to a reaction. All molecules or ions with a free pair of electrons or at least one pi bond can act as nucleophiles. Because nucleophiles donate electrons, they are by definition Lewis bases. In one embodiment of the present disclosure, a nucleophile may be either a sulfur nucleophile or a nitrogen nucleophile.

The term “sulfur nucleophile,” as used herein, refers to a nucleophile comprising at least one sulfur atom. The example of sulfur nucleophile may include hydrogen sulfide and its salts, thiols (RSH), thiolate anions (RS—), anions of thiolcarboxylic acids (RC(O)—S—), and anions of dithiocarbonates (RO—C(S)—S—) and dithiocarbamates (R 2N—C(S)—S—). In one embodiment of the present disclosure, the sulfur nucleophile is MESNA or DTT.

The term “nitrogen nucleophile,” as used herein, refers to a nucleophile comprising at least one nitrogen atom. Nitrogen nucleophiles include ammonia, azide, amines, hydrazines, and nitrites. In one embodiment of the present disclosure, the nitrogen nucleophile is hydrazine.

The term “exogenous nucleophile”, as used herein, means that the nucleophile does not form part of the complex of this disclosure or of the split intein C-fragment.

Thus, in the present method, wherein the compound of interest is a protein or a polypeptide, the intein intermediate is reacted with a nucleophile to release the polypeptide of interest from the bound intein N- and C-fragments thereby obtaining a protein or polypeptide having a C-terminus modified by the nucleophile. The type of modification will depend on the type of nucleophile. For example, when the nucleophile is a thiol, the modified polypeptide of interest is an α-thioester, which in turn can be further modified, e.g., with a different nucleophile (e.g., a drug, a polymer, another polypeptide, a oligonucleotide), or any other moiety using the well-known α-thioester chemistry for protein modification at the C-terminus. One advantage of this chemistry is that only the C-terminus is modified with a thioester for further modification, thus allowing for selective modification only at the C terminus and not at any other acidic residue in the polypeptide. In the case wherein the compound of interest is not a protein or a polypeptide the compound of interest will carry a moiety able to react with the nucleophile, that is, an electrophile. Suitable electrophiles capable to react with a nucleophile are commonly known in the field.

In certain embodiments, the nucleophile is added to the reaction after contacting the first complex of this disclosure and the split intein C-fragment. In another embodiment, the first complex of this disclosure, the split intein C-fragment and the nucleophile are contacted simultaneously.

In certain embodiments, the method further comprises contacting the conjugate of the compound of interest and the nucleophile with a second exogenous nucleophile.

The nucleophile that is used in the methods disclosed herein either with the intein intermediate or as a subsequent or second nucleophile reacting with, e.g., an α-thioester, can be any compound or material having a suitable nucleophilic moiety. For example, to form a thioester, a thiol moiety is contemplated as the nucleophile. In some cases, the thiol is a 1,2 aminothiol, or a 1,2-aminoselenol. An α-selenothioester can be formed by using a selenothiol (R-SeH). Alternative nucleophiles contemplated include amines (i.e. aminolysis to give amides directly), hydrazines (to give hydrazides), amino-oxy groups (to give hydroxamic acids). Additionally, the nucleophile can be a functional group within a compound of interest for conjugation to the polypeptide of interest (e.g., a drug to form a protein-drug conjugate) or could alternatively bear an additional functional group for subsequent known bioorthogonal reactions such as an azide or an alkyne (for a click chemistry reaction between the two function groups to form a triazole), a tetrazole, an α-ketoacid, an aldehyde or ketone, or a cyanobenzothiazole.

In certain embodiments, the split intein N-fragment comprises or consists of a sequence selected from the group consisting of SEQ ID NO: 111-113.

In certain embodiments, the split intein N-fragment comprises or consists of a sequence selected from the group consisting of SEQ ID NO: 49-68 or a functionally equivalent variant thereof.

### Composition Comprising Polynucleotides

In another aspect, this disclosure relates to a composition, hereinafter second composition of this disclosure, comprising:

(a) a first polynucleotide encoding a first fusion protein comprising, from the N-terminus to the C-terminus:
a first polypeptide of interest and
a split intein N-fragment comprising the sequence of SEQ ID NO: 1 or a variant thereof having at least 90% sequence identity with SEQ ID NO: 1, or an amino acid sequence selected from the group consisting of SEQ ID NO: 103-110 and
   (b) a second polynucleotide encoding a second fusion protein comprising, from the N-terminus to the C-terminus:
an AceL-TerL split intein C-fragment or a variant thereof or a split intein C-fragment comprising the sequence of SEQ ID NO: 7 or a variant thereof having at least 88% sequence identity with SEQ ID NO: 7 or an amino acid sequence selected from the group consisting of SEQ ID NO: 114-120 and
a second polypeptide of interest
   or
(a) a first polynucleotide encoding a first fusion protein comprising, from the N-terminus to the C-terminus:
   a first polypeptide of interest and
   an AceL-TerL split intein N-fragment or a variant thereof, or a split intein N-fragment comprising the sequence of SEQ ID NO: 1 or a variant thereof having at least 90% sequence identity with SEQ ID NO: 1, or an amino acid sequence selected from the group consisting of SEQ ID NO: 103-110 and
(b) a second polynucleotide encoding a second fusion protein comprising, from the N-terminus to the C-terminus:
   a split intein C-fragment comprising the sequence of SEQ ID NO: 7 or a variant thereof having at least 88% sequence identity with SEQ ID NO: 7 or an amino acid sequence selected from the group consisting of SEQ ID NO: 114-120 and
   a second polypeptide of interest.

In certain embodiments, the variants are functionally equivalent variants.

The term “composition” has been previously defined. In certain embodiments, the first polynucleotide is packed together with the second polynucleotide in a single formulation. In another embodiment, the first polynucleotide and of the second polynucleotide are separately packed.

The term “AceL-TerL intein” has been previously defined. In certain embodiments, the AceL-TerL split intein N-fragment comprises or consists on the sequence of SEQ ID NO: 101 or 102. In certain embodiments, the AceL-TerL split intein C-fragment comprises or consists on the sequence of SEQ ID NO: 99 or 100.

In certain embodiments, the first polypeptide of interest is the N-terminal fragment of a protein and the second polypeptide of interest is the C-terminal fragment of said protein; in certain embodiments a protein of more than 25 KDa, more than 50 KDa or more than 100 KDa, such that upon covalently linking the C-terminus of the first polypeptide of interest to the N-terminus of the second polypeptide of interest the whole protein is obtained.

In some embodiments the first compound and second compound is or includes an antibody, antibody chain, or antibody heavy chain. In certain embodiments, the polypeptide of interest is an antibody or a fragment of an antibody. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDEC-205 monoclonal antibody, as described by Stevens et al., JACS 2016, 138: 2162-5.

In certain embodiments, the split intein N-fragment comprises or consists of a sequence selected from the group consisting of SEQ ID NO: 111-113.

In certain embodiments, the split intein N-fragment comprises or consists of a sequence selected from the group consisting of SEQ ID NO: 49-68 or a functionally equivalent variant thereof.

In certain embodiments, the split intein C-fragment comprises or consists of a sequence selected from the group consisting of SEQ ID NO: 121-124.

In certain embodiments, the split intein C-fragment comprises or consists of a sequence selected from the group consisting of SEQ ID NO: 69-87 or a functionally equivalent variant thereof.

In certain embodiments, the split intein N-fragment comprises or consists of a sequence selected from the group consisting of SEQ ID NO: 49-68 or a functionally equivalent variant thereof and the split intein C-fragment comprises or consists of a sequence selected from the group consisting of SEQ ID NO: 69-87 or a functionally equivalent variant thereof.

The second composition of this disclosure can be used for expressing a gene of interest in a cell using the method of this disclosure.

### Methods for Expressing a Gene of Interest

In another aspect, this disclosure relates to a method for expressing a gene of interest in a cell, hereinafter first method for expressing a gene of interest, comprising:

(i) contacting the cell with
(a) a first polynucleotide encoding a first fusion protein comprising, from the N-terminus to the C-terminus:
a first polypeptide of interest and
a split intein N-fragment comprising the sequence of SEQ ID NO: 1 or a functionally equivalent variant thereof having at least 90% or an amino acid sequence selected from the group consisting of SEQ ID NO: 103-110, and
   (b) a second polynucleotide encoding a second fusion protein comprising, from the N-terminus to the C-terminus:
an AceL-TerL split intein C-fragment or a functionally equivalent variant thereof or a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
second polypeptide of interest,
   or
   (a) a first polynucleotide encoding a first fusion protein comprising, from the N-terminus to the C-terminus:
a first polypeptide of interest and
an AceL-TerL split intein N-fragment or a functionally equivalent variant thereof or a split intein N-fragment comprising the sequence of SEQ ID NO: 1 or a functionally equivalent variant thereof having at least 90% or an amino acid sequence selected from the group consisting of SEQ ID NO: 103-110, and
   (b) a second polynucleotide encoding a second fusion protein comprising, from the N-terminus to the C-terminus:
a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
second polypeptide of interest,
   (ii) allowing the expression of the first and the second polynucleotides so that the first and the second fusion proteins are produced and
   (iii) allowing the contact between the first and second fusion proteins so that the split intein N-fragment binds to the split intein C-fragment to form a intein intermediate and the intein intermediate reacts to covalently link the C-terminus of the first polypeptide of interest to the N-terminus of the second polypeptide of interest.

In another aspect, this disclosure relates to a method for expressing a gene of interest, hereinafter second method for expressing a gene of interest of this disclosure, comprising:
(i) contacting a first cell with a first polynucleotide encoding a first fusion protein comprising, from the N-terminus to the C-terminus:
   a first polypeptide of interest and
   a split intein N-fragment comprising the sequence of SEQ ID NO: 1 or a functionally equivalent variant thereof having at least 90% or an amino acid sequence selected from the group consisting of SEQ ID NO: 103-110,
wherein the first fusion protein comprises a signal peptide, and
(ii) contacting a second cell with a second polynucleotide encoding a second fusion protein comprising, from the N-terminus to the C-terminus:
   an AceL-TerL split intein C-fragment or a functionally equivalent variant thereof or a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
   second polypeptide of interest
wherein the second fusion protein comprises a signal peptide,
or
(i) contacting a first cell with a first polynucleotide encoding a first fusion protein comprising, from the N-terminus to the C-terminus:
   a first polypeptide of interest and
   an AceL-TerL split intein N-fragment or a functionally equivalent variant thereof or a split intein N-fragment comprising the sequence of SEQ ID NO: 1 or a functionally equivalent variant thereof having at least 90% or an amino acid sequence selected from the group consisting of SEQ ID NO: 103-110,
wherein the first fusion protein comprises a signal peptide, and
(ii) contacting a second cell with a second polynucleotide encoding a second fusion protein comprising, from the N-terminus to the C-terminus:
   a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
   second polypeptide of interest
wherein the second fusion protein comprises a signal peptide,
(iii) allowing the expression of the first and the second polynucleotides so that the first and the second fusion proteins are produced and secreted,
(iv) allowing the contact between the first and second fusion proteins so that the split intein N-fragment binds to the split intein C-fragment to form a intein intermediate and the intein intermediate reacts to covalently link the C-terminus of the first polypeptide of interest to the N-terminus of the second polypeptide of interest.

The term “AceL-TerL intein” has been previously defined. In certain embodiments, the AceL-TerL split intein N-fragment comprises or consists on the sequence of SEQ ID NO: 101 or 102. In certain embodiments, the AceL-TerL split intein C-fragment comprises or consists on the sequence of SEQ ID NO: 99 or 100.

In certain embodiments, the first polypeptide of interest is the N-terminal fragment of a protein and the second polypeptide of interest is the C-terminal fragment of said protein; in certain embodiments a protein of more than 25 KDa, more than 50 KDa or more than 100 KDa, so that upon covalently linking the C-terminus of the first polypeptide of interest to the N-terminus of the second polypeptide of interest the whole protein is obtained.

In certain embodiments, the first or second polypeptide of interest is Cas9 or a fragment of Cas9. In certain embodiments, the first polypeptide of interest is an N-terminal fragment of Cas9, and the second polypeptide of interest is a C-terminal fragment of Cas9. In another embodiment, when the first polypeptide of interest is an N-terminal fragment of Cas9 and the second polypeptide of interest is a C-terminal fragment of Cas9, upon covalently linking the C-terminus of the N-terminal fragment of Cas9 to the N-terminus of the C-terminal fragment of Cas9, the whole Cas9 protein is obtained

In some embodiments the first compound and/or the second compound is or includes an antibody, an antibody fragment, an antibody chain, or antibody heavy chain. In certain embodiments, the polypeptide of interest is the heavy chain of an anti-DEC-205 antibody. In certain embodiments, the polypeptide of interest is the heavy chain of an anti-DEC-205 monoclonal antibody. In certain embodiments, the compound of interest is the heavy chain of the mouse αDEC-205 monoclonal antibody, as described by Stevens et al., JACS 2016, 138: 2162-5.

In certain embodiments, the split intein N-fragment comprises or consists of a sequence selected from the group consisting of SEQ ID NO: 111-113.

In certain embodiments, the split intein N-fragment comprises or consists of a sequence selected from the group consisting of SEQ ID NO: 49-68 or a functionally equivalent variant thereof.

In certain embodiments, the split intein C-fragment comprises or consists of a sequence selected from the group consisting of SEQ ID NO: 121-124.

In certain embodiments, the split intein C-fragment comprises or consists of a sequence selected from the group consisting of SEQ ID NO: 69-87 or a functionally equivalent variant thereof.

In certain embodiments, the split intein N-fragment comprises or consists of a sequence selected from the group consisting of SEQ ID NO: 49-68 or a functionally equivalent variant thereof and the split intein C-fragment comprises or consists of a sequence selected from the group consisting of SEQ ID NO: 69-87 or a functionally equivalent variant thereof.

The contacting of the cell with the first and/or second polynucleotide can be made by any suitable means for allowing introducing a polynucleotide of interest into a cell, for example, transfection, electroporation, microinjection, transduction, lipofection, cell fusion, DEAE dextran, calcium phosphate precipitation, use of a gene gun, or use of a DNA vector transporter. In the first method for expressing a gene of interest of this disclosure, it is contemplated that the cell is contacted simultaneously with the first and second polynucleotide, or sequentially with the first and second polynucleotide in any order, that is, the cell can be contacted firstly with the first polynucleotide and secondly with the second polynucleotide or firstly with the second polynucleotide and secondly with the first polynucleotide.

Any cell previously defined as a host cell can be used in these methods.

The term “signal peptide” or “secretory signal peptide”, as used herein, refers to a peptide of a relatively short length, generally between 5 and 30 amino acid residues, directing proteins synthesized in the cell towards the secretory pathway. The signal peptide usually contains a series of hydrophobic amino acids adopting a secondary alpha helix structure. Additionally, many peptides include a series of positively-charged amino acids that can contribute to the protein adopting the suitable topology for its translocation. The signal peptide tends to have at its carboxyl end a motif for recognition by a peptidase, which is capable of hydrolyzing the signal peptide giving rise to a free signal peptide and a mature protein. The signal peptide can be cleaved once the protein of interest has reached the appropriate location. Any secretory signal peptide may be used in the present disclosure.

In certain embodiments, the signal peptide is linked to the N-terminus of the first polypeptide of interest in the first fusion protein.

In certain embodiments, the signal peptide is linked to the N-terminus of the split intein C-fragment in the second fusion protein.

The invention will be described by way of the following examples which are to be considered as merely illustrative and not limitative of the scope of this disclosure.

### EXAMPLES

### Materials and Methods

### Materials

Oligonucleotides and synthetic genes were purchased from Integrated DNA Technologies (Coralville, Iowa). Pfu Ultra II Hotsart fusion polymerase for cloning was purchased from Agilent (La Jolla, Calif.). All restriction enzymes and 2× Gibson Assembly Master Mix were purchased from New England Biolabs (Ipswich, Mass.). High-competency cells used for cloning and protein expression were generated from One Shot BI21 (DE3) chemically competent E. coli and sub-cloning efficiency DH5a competent cells purchased from Invitrogen (Carlsbad, Calif.). DNA purification kits were purchased from Qiagen (Valencia, Calif.). All plasmids were sequenced by GENEWIZ (South Plainfield, N.J.). Luria Bertani (LB) media, and all buffering salts were purchased from Fisher Scientific (Pittsburgh, Pa.). Dimethylformamide (DMF), dichloromethane (DCM), Coomassie brilliant blue, triisopropylsilane (TIS), β-mercaptoethanol (BME), DL-dithiothreitol (DTT), sodium 2-mercaptoethanesulfonate (MESNa), 5(6)-carboxyfluorescein, and thermolysin were purchased from Sigma-Aldrich (Milwaukee, Wis.). Tris (2-carboxyethyl) phosphine hydrochloride (TCEP) and isopropyl-8-D-thiogalactopyranoside (IPTG) were purchased from Gold Biotechnology (St. Louis, Mo.). Roche Complete Protease Inhibitors were used for protein purification (Roche, Branchburg, N.J.). Nickel-nitrilotriacetic acid (Ni-NTA) resin was purchased from Thermo scientific (Rockford, Ill.). Fmoc amino acids were purchased from Novabiochem (Darmstadt, Germany) or Bachem (Torrance, Calif.). O-(Benzotriazol-1-yl)-N,N,N′,N′-tetramethyluronium hexafluorophosphate (HBTU) was purchased from Genscript (Piscataway, N.J.). Trifluoroacetic acid (TFA) was purchased from Halocarbon (North Augusta, S.C.). MES-SDS running buffer was purchased from Boston Bioproducts (Ashland, Mass.).

### Equipment

Analytical reverse phase high performance liquid chromatography (RP-HPLC) was carried out on Hewlett-Packard 1100 and 1200 series instruments equipped with a C18 Vydac column (5 μm, 4.6×150 mm). All HPLC runs used the following solvents at a flow rate of 1 mL/min: 0.1% TFA (trifluoroacetic acid) in water (solvent A) and 90 acetonitrile in water with 0.1% TFA (solvent B). All peptides and proteins were analyzed using the gradient: 0% B for 2 min followed by 0-73% B for 30 min. Electrospray ionization mass spectrometric analysis (ESI-MS) was carried out on a Bruker Daltonics MicroTOF-Q II mass spectrometer. Size-exclusion chromatography (SEC) was performed on an AKTA FPLC system (GE Healthcare) with a Superdex S75 16/60 column (125 mL column volume) for preparative runs and a Superdex S75 10/300 column for analytical runs. Gels were imaged with a LI-COR Odyssey Infrared Imager. Circular dichroism experiments were carried out on a Chirascan Circular Dichroism spectrometer (Applied Photophysics). Cell lysis was carried out using a S-450D Branson Digital Sonifier. NMR experiments were carried out on a Bruker 900, 800, 600 and 500 MHz spectrometers with 5 mm TCI triple resonance cryoprobes. Steady state fluorescence measurements were performed on a Horiba Flourmax 4 fluorimeter. Stopped flow anisotropy measurements were performed on an Applied Photophysics SX20 stopped-flow spectrometer.

### Consensus Protein Design

Homologues of AceL TerL were identified through a BLAST search of metagenomic data in the NCBI (nucleotide collection) and JGI databases using the TerL DNA sequence. This led to the identification of TerL N- and C-inteins with high sequence identity to AceL (Table 1). Because the cognate N- and C-inteins could not been matched, the split inteins were treated as two distinct datasets and analyzed separately. MSAs of these split inteins were then generated in Jalview⁴, and the consensus sequence was determined. At some positions in the N-intein, additional residues from the alignment corresponding to loops not present in AceL were included in the consensus sequence.

TABLE 1
Identified TerL Inteins

| | | | | SEQ ID |
|---|---|---|---|---|
| | Source | Project # | Sequence | NO |
| C-intein | | | | |
| AceL | Thiel et | | MFRTNTNNIKILSPNGFSNFNGIQKVERNLY | 99 |
| | al¹ | | QHIIFDDDTEIKTSINHPFGKDKILARDVKV | |
| | | | GDYLNSKKVLYNELVNENIFLYDPINVEKES | |
| | | | LYITNGVVSHN | |
| AceL* | Thiel et | | MFRTNTNNIKILGPNGFSNFIGIQKVERDQY | 100 |
| | al¹ | | QHIIFDDDTEIKTSINHPFGKDKILARDVKV | |
| | | | GDYLNSKKVLYNELVNENIFLYDPINVEKES | |
| | | | LYITNGVVSHN | |
| Cep | NCBI | CEPX01183120 | MFKTNTNNIKILSPDGFSNFNGIQKVKRKLY | 69 |
| | | | QHIIFEGGTEIKTSINHSFGKDKILARDIKV | |
| | | | GDYLNNKKVLYNELVNEKIFLYDPINVEKEN | |
| | | | LYITNDVVSHN | |
| Mdt | NCBI | MDTC01246584 | MYKVNNNIKVKTPTGFQSFSGIQKVFKPFYH | 70 |
| | | | WIIFDDGSEIKCSDNHSFGSEKIKASSLKLD | |
| | | | DIIQGKKVLYNEIVEEGIYLYDLLDVGEENL | |
| | | | YITNKIISHN | |
| Meh | NCBI | MEHZ010888690 | MSKTYEVLSPSGFVKFSGIQKVSRSKYRHFI | 71 |
| | | | FDDGAELKCSLNHRFGKDEILASSLWPSSDL | |
| | | | QGKNILYAEDVEEDIDLYDLLNVGGGNLYYT | |
| | | | NGLVSHN | |
| Aac | NCBI | AACY020064168 | MFKINKNIKVKTPDGFKDFSGIQKVYKPFYH | 72 |
| | | | WIIFDDGSEIKCSDNHSFGKEKIKASTIKVD | |
| | | | DILQEKKVLYNEIVEEGIYLYDLLDVGEDNL | |
| | | | YYSNNIVSHN | |
| Aac2 | NCBI | AACY023445674 | MFKLNKNIEVKTPDGFKSFSGIQKVYKPFYH | 73 |
| | | | WIIFDDGSEIKCSDNHSFGKEKIKASTIRVD | |
| | | | DFLQGKKVVYNEIVEEGIYLYDLLDVGENNL | |
| | | | YYSNNIISHN | |
| Cen | NCBI | CENI01048299 | MYKLNSSIKVKTPRGFKKFAGIQKVRKPVYQ | 74 |
| | | | WIIFGDDSEIKCSLDHSFGEEQVKAHTIKTG | |
| | | | DLLQHKEVVYSEIVEEPIDLYDLLEVEDGNL | |
| | | | YNTNGVVSHN | |
| Cep2 | NCBI | CEPQ01016765 | MYEVLSPSGFVKFSGVQKVSRSKYRHFIFDD | 75 |
| | | | GTEIKCSLDHRFGGLWDEDEILASSLNRGEY | |
| | | | LQGKKILYVEDVEEQIDLYDLMNVDGGNLYY | |
| | | | TNGLVSHN | |
| Cep3 | NCBI | CEPZ01087314 | MSKTYEVLSPSGFVKFSGIQKVSHSKYRHFI | 76 |
| | | | FDDGTELKCSFNHRFGKDEILASSLCRGSDL | |
| | | | QGKKILYAEDVEEDIDLYDLLNVGGGNLYYT | |
| | | | NGLVSHN | |
| Cep4 | NCBI | CEPZ01013308 | MYIRYQKTTSKTYEVLSPSGFVNFSGIQTVP | 77 |
| | | | HSKYRHFIFDDGTELKCSLNHRFDKDEILAS | |
| | | | SLWRGAELQGKQILYAEDIEEDIDLYDLLNV | |
| | | | GGGNLYYTNGLVSHN | |
| Cep5 | NCBI | CEPS01165861 | MFTKYKILTPNGYESFDGVNRIKRDMYSHLI | 78 |
| | | | FSSGIEIRCSLNHPLYISKGDIIKSYELKIG | |
| | | | DKVLSKNGWEIVTYNEIIEEPIYLYDIINSG | |
| | | | KDHNYYTNDILSHN | |
| Cep6 | NCBI | CEPZ01055800 | MISKNFTKYKILTPNGYESFDGVNRIKRDMY | 79 |
| | | | SHLIFSSGIEIRCSLNHPLYISKGDIIKSYE | |
| | | | LKIGDKVLSKNGWEIVTYNEIIEEPIYLYDI | |
| | | | INSGKDHNYYTNDILSHN | |
| Lak | JGI | Ga0169931 | MFKLNKNIEVKTPDGFKSFSGIQKVYKPFYH | 80 |
| | | | WIIFDDGSEIKCSDNHSFGKEKIKASTIRVD | |
| | | | DFLQGKKVVYNEIVEEGIYLYDLLDVGENNL | |
| | | | YYSNDIISHN | |
| Kab | JGI | Ga0172376 | MFKLNKNIRVKTPSGFKSFSGIQKVYKPFYH | 81 |
| | | | WIIFDDGSEIKCSDNHSFGEEQIKASSIKVD | |
| | | | DFLQGKKVVYNEIVEEGIYLYDLLDVGEDNL | |
| | | | YYSNDVVSHN | |
| Chb | JGI | Ga0129336 | MFKLNKNIKVKTPRGFKFFSGIQKVYKPYYH | 82 |
| | | | WIIFDDGSEIKCSDNHSFGKEKTKASTIKVD | |
| | | | DFLQGKKVVYNEIVEEGIYLYDLLDVGEDNL | |
| | | | YYSNEIISHN | |
| Del | JGI | Ga0075462 | MFKLNKNIKVKTPSGFKYFSGIQKVYKPFYH | 83 |
| | | | WIIFDDGTEIKCSDNHSFGKEQIKASMIKVD | |
| | | | DFFQGKKVVYNEIVEEEIYLYDLLDVGEDNL | |
| | | | YFSNGIISHN | |
| Del2 | JGI | Ga0075478 | MFKLNKNIEVKTPDGFKSFSGIQKVYKPFYH | 84 |
| | | | WIIFDDGSEIKCSDNHSFGKEKIKASTIKVD | |
| | | | DLLQGKKVVYNEIVEEGIYLYDLLDVGEDNL | |
| | | | YYSNNLVSHN | |
| Del3 | JGI | Ga0075478 | MFKLNKNITVKTPSGFKSFSGIQKVYKPFYH | 85 |
| | | | WIIFDDGSEIKCSDNHSFGEEQIKASMIKVD | |
| | | | DFLQGKKVVYNEVVEEGVYLYDLLDVGEDNL | |
| | | | YYSNNIISHN | |
| AceL2 | JGI | Ga0075117 | MFRTNTDNIKILSPSGFSNFNGIQKVERDLY | 86 |
| | | | QHIIFDDKSEIKTSINHPFGKDKILARNIKV | |
| | | | GDYLNSKKVLYNELVAEKITLYDPINVEKEN | |
| | | | LYITNGVISHN | |
| AceL3 | JGI | Ga0075117 | MFRTNTDNIKILSPSGFSNFNGIQKVERDLY | 87 |
| | | | QHIIFDDKSEIKTSINHPFGKDKILARNIKV | |
| | | | GDYLNSKKVLYNELVNEKITLYDPINVEKEN | |
| | | | LYITNGVISHN | |
| N-intein | | | | |
| AceL | Thiel et | | CVYGDTMVETEDGKIKIEDLYKRLA | 101 |
| | al¹ | | | |
| AceL* | Thiel et | | CVSGDTMVETEDGKIKIEDLYKRLA | 102 |
| | al¹ | | | |
| AAC | NCBI | AACY023445674 | CLGGDTIIEIQDDDGITQKISMEDLYERL | 49 |
| AAC2 | NCBI | AACY020064168 | CLGGDTEIEILDDNGIVQKTSMENLYERL | 50 |
| FUW | NCBI | FUWD010114546 | CLGGETLIEIQDDNENISKVSMEDLYDRM | 51 |
| FUW2 | NCBI | FUWD010387041 | CVDGDTIVEIYDKKTKEEYCVKIKDLYDLI | 52 |
| FUW3 | NCBI | FUWD012964875 | CLSGDTQIEIKNVNDKIESVSMEELYERM | 53 |
| AAC3 | NCBI | AACY020820060 | CLSGDTMIEILDENGIPQKISMEDLYQR | 54 |
| MDT | NCBI | MDTB01192700 | CVSGDTNIEIECEDGVETTTIKDLYDRM | 55 |
| CEP | NCBI | CEPX01183120 | CVDGDTMVETEDGKIKIEDLYKKL | 56 |
| MEH | NCBI | MEHZ011579446 | CVYGDTMVETEDGKIKIEDLYKKL | 57 |
| MDT2 | NCBI | MDTC01246584 | CVRGDTLVEVEKDDVISEMRIEDLYNRM | 58 |
| ABL | NCBI | ABLX01341501 | CVGGNTLVEVEKDDIISEMRIEDLYNTM | 59 |
| SSF | JGI | Ga0102963 | CLSGDTTIEILDVDGIPQKISMEDLYQRL | 60 |
| Del | JGI | Ga0075478_10047284 | CLSGDTMIEILDESGIPQKISMKELYQRM | 61 |
| Del2 | JGI | Ga0075478_10000264 | CLDGNTSIEILDENNTIQKISMENLYKRL | 62 |
| Del3 | JGI | Ga0070746 | CLGGDTIIEIQDDDGITQKISMEDLYQRL | 63 |
| Del4 | JGI | Ga0070752 | CLDGGTSIEILDTNNITQKISLENLYERL | 64 |
| Del5 | JGI | Ga0070749 | CLSGDTSIEILDENNTIQKISMEDLYERL | 65 |
| Del6 | JGI | Ga0070754 | CLSGDTLIEIIDDDGNTQKISMEDLY | 66 |
| Del7 | JGI | Ga0070751 | CLSGDTLIEIIDDDGNTQKISMEDLYQ | 67 |
| Kab | JGI | Ga0172375 | CLGGDTIIEIKDDDGITQKISMEDLYQRL | 68 |

¹Angewandte Chemie International Edition 2014, 53(5): 1306-1310

### Cloning of Recombinant DNA

Synthetic genes were purchased and introduced into pET-30 expression vectors using Gibson assembly. Targeted mutations were introduced using inverse PCR with Pfu Ultra II HF Polymerase. The identity of all recombinant plasmids was confirmed through sequencing and the corresponding protein sequences are reported in Table 2.

TABLE 2
Sequence of proteins utilized in the present application.

| Construct | ^{a}Sequence | SEQ ID NO |
|---|---|---|
| SUMO-Cat^{N} | MGSSHHHHHHGSGLVPRGSASMSDSEVNQEAKPEVKPEVKPETHINLK | 88 |
| | VSDGSSEIFFKIKKTTPLRRLMEAFAKRQGKEMDSLRFLYDGIRIQAD | |
| | QTPEDLDMEDNDIIEAHREQIGGEFEALSGDTMIEILDDDGIIQKISM | |
| | EDLYQRLA | |
| SUMO-AceL*^{N} | MGSSHHHHHHGSGLVPRGSASMSDSEVNQEAKPEVKPEVKPETHINLK | 89 |
| | VSDGSSEIFFKIKKTTPLRRLMEAFAKRQGKEMDSLRFLYDGIRIQAD | |
| | QTPEDLDMEDNDIIEAHREQIGGEFEAVSGDTMVETEDGKIKIEDLYK | |
| | RLA | |
| SUMO-GOS^{N} | MGSSHHHHHHGSGLVPRGSASMSDSEVNQEAKPEVKPEVKPETHINLK | 90 |
| | VSDGSSEIFFKIKKTTPLRRLMEAFAKRQGKEMDSLRFLYDGIRIQAD | |
| | QTPEDLDMEDNDIIEAHREQIGGEFEAISQESYINIEVNGKVETIKIG | |
| | DLYKKLSFNERKFNE | |
| SUMO-Cat^{C} | MGSSHHHHHHGSGLVPRGSASMSDSEVNQEAKPEVKPEVKPETHINLK | 91 |
| | VSDGSSEIFFKIKKTTPLRRLMEAFAKRQGKEMDSLRFLYDGIRIQAD | |
| | QTPEDLDMEDNDIIEAHREQIGGMFKLNTKNIKVLTPSGFKSFSGIQK | |
| | VYKPFYHHIIFDDGSEIKCSDNHSFGKDKIKASTIKVGDYLQGKKVLY | |
| | NEIVEEGIYLYDLLNVGEDNLYYTNGIVSHACEFL | |
| ^{b}SUMO-Flag-Cat^{C} | MGSSHHHHHHGSGLVPRGSASMSDSEVNQEAKPEVKPEVKPETHINLK | 92 |
| | VSDGSSEIFFKIKKTTPLRRLMEAFAKRQGKEMDSLRFLYDGIRIQAD | |
| | QTPEDLDMEDNDIIEAHREQIGGDYKDDDDKMFKLNTKNIKVLTPSGF | |
| | KSFSGIQKVYKPFYHHIIFDDGSEIKCSDNHSFGKDKIKASTIKVGDY | |
| | LQGKKVLYNEIVEEGIYLYDLLNVGEDNLYYTNGIVSHACESRGK | |
| SUMO-AceL*^{C} | MGSSHHHHHHGSGLVPRGSASMSDSEVNQEAKPEVKPEVKPETHINLK | 93 |
| | VSDGSSEIFFKIKKTTPLRRLMEAFAKRQGKEMDSLRFLYDGIRIQAD | |
| | QTPEDLDMEDNDIIEAHREQIGGMFRTNTNNIKILGPNGFSNFIGIQK | |
| | VERDQYQHIIFDDDTEIKTSINHPFGKDKILARDVKVGDYLNSKKVLY | |
| | NELVNENIFLYDPINVEKESLYITNGVVSHACEFL | |
| SUMO-GOS^{C} | MGSSHHHHHHGSGLVPRGSASMSDSEVNQEAKPEVKPEVKPETHINLK | 94 |
| | VSDGSSEIFFKIKKTTPLRRLMEAFAKRQGKEMDSLRFLYDGIRIQAD | |
| | QTPEDLDMEDNDIIEAHREQIGGMKLPESVVKNNINLKIETPYGFENF | |
| | YGVNKIKKDKYIHLEFTNGEKLKCSLDHPLSTIDGIVKAKDLDKYTEV | |
| | YTKFGGCFLKKSKVINESIELYDIVNSGLKHLYYSNNIISHACEFL | |
| AceL*^{C}-GFP | MGSSHHHHHHGSGLVPRGSASMSDSEVNQEAKPEVKPEVKPETHINLK | 95 |
| | VSDGSSEIFFKIKKTTPLRRLMEAFAKRQGKEMDSLRFLYDGIRIQAD | |
| | QTPEDLDMEDNDIIEAHREQIGGMFRTNTNNIKILGPNGFSNFIGIQK | |
| | VERDQYQHIIFDDDTEIKTSINHPFGKDKILARDVKVGDYLNSKKVLY | |
| | NELVNENIFLYDPINVEKESLYITNGVVSHNCEFLMVSKGEELFTGVV | |
| | PILVELDGDVNGHKFSVSGEGEGDATYGKLTLKFICTTGKLPVPWPTL | |
| | VTTLTYGVQCFSRYPDHMKQHDFFKSAMPEGYVQERTIFFKDDGNYKT | |
| | RAEVKFEGDTLVNRIELKGIDFKEDGNILGHKLEYNYNSHNVYIMADK | |
| | QKNGIKVNFKIRHNIEDGSVQLADHYQQNTPIGDGPVLLPDNHYLSTQ | |
| | SALSKDPNEKRDHMVLLEFVTAAGITLGMDELYKDYKDDDDK | |
| ^{c}Cat^{C}-GFP | MGSSHHHHHHGSGLVPRGSASMSDSEVNQEAKPEVKPEVKPETHINLK | 96 |
| | VSDGSSEIFFKIKKTTPLRRLMEAFAKRQGKEMDSLRFLYDGIRIQAD | |
| | QTPEDLDMEDNDIIEAHREQIGGMFKLNTKNIKVLTPSGFKSFSGIQK | |
| | VYKPFYHHIIFDDGSEIKCSDNHSFGKDKIKASTIKVGDYLQGKKVLY | |
| | NEIVEEGIYLYDLLNVGEDNLYYTNGIVSHNC [symbol] LMVSKGEELFTGVV | |
| | PILVELDGDVNGHKFSVSGEGEGDATYGKLTLKFICTTGKLPVPWPTL | |
| | VTTLTYGVQCFSRYPDHMKQHDFFKSAMPEGYVQERTIFFKDDGNYKT | |
| | RAEVKFEGDTLVNRIELKGIDFKEDGNILGHKLEYNYNSHNVYIMADK | |
| | QKNGIKVNFKIRHNIEDGSVQLADHYQQNTPIGDGPVLLPDNHYLSTQ | |
| | SALSKDPNEKRDHMVLLEFVTAAGITLGMDELYKDYKDDDDK | |
| MBP-AceL*^{N} | MGSSHHHHHHGSGLVPRGSASMSDSEVNQEAKPEVKPEVKPETHINLK | 97 |
| | VSDGSSEIFFKIKKTTPLRRLMEAFAKRQGKEMDSLRFLYDGIRIQAD | |
| | QTPEDLDMEDNDIIEAHREQIGGKIEEGKLVIWINGDKGYNGLAEVGK | |
| | KFEKDTGIKVTVEHPDKLEEKFPQVAATGDGPDIIFWAHDRFGGYAQS | |
| | GLLAEITPDKAFQDKLYPFTWDAVRYNGKLIAYPIAVEALSLIYNKDL | |
| | LPNPPKTWEEIPALDKELKAKGKSALMFNLQEPYFTWPLIAADGGYAF | |
| | KYENGKYDIKDVGVDNAGAKAGLTFLVDLIKNKHMNADTDYSIAEAAF | |
| | NKGETAMTINGPWAWSNIDTSKVNYGVTVLPTFKGQPSKPFVGVLSAG | |
| | INAASPNKELAKEFLENYLLTDEGLEAVNKDKPLGAVALKSYEEELAK | |
| | DPRIAATMENAQKGEIMPNIPQMSAFWYAVRTAVINAASGRQTVDEAP | |
| | KDAQTNEFECVSGDTMVETEDGKIKIEDLYKRLA | |
| ^{c}MBP-Cat^{N} | MGSSHHHHHHGSGLVPRGSASMSDSEVNQEAKPEVKPEVKPETHINLK | 98 |
| | VSDGSSEIFFKIKKTTPLRRLMEAFAKRQGKEMDSLRFLYDGIRIQAD | |
| | QTPEDLDMEDNDIIEAHREQIGGKIEEGKLVIWINGDKGYNGLAEVGK | |
| | KFEKDTGIKVTVEHPDKLEEKFPQVAATGDGPDIIFWAHDRFGGYAQS | |
| | GLLAEITPDKAFQDKLYPFTWDAVRYNGKLIAYPIAVEALSLIYNKDL | |
| | LPNPPKTWEEIPALDKELKAKGKSALMFNLQEPYFTWPLIAADGGYAF | |
| | KYENGKYDIKDVGVDNAGAKAGLTFLVDLIKNKHMNADTDYSIAEAAF | |
| | NKGETAMTINGPWAWSNIDTSKVNYGVTVLPTFKGQPSKPFVGVLSAG | |
| | INAASPNKELAKEFLENYLLTDEGLEAVNKDKPLGAVALKSYEEELAK | |
| | DPRIAATMENAQKGEIMPNIPQMSAFWYAVRTAVINAASGRQTVDEAP | |
| | KDAQTNE [symbol] CLSGDTMIEILDDDGIIQKISMEDLYQRLA | |
| Fl-Cat^{N} | Fl-GEFEALSGDTMIEILDDDGIIQKISMEDLYQRLA | 167 |

^{a}The sequences shown correspond to the complete protein expressed by the pET-30 expression vector. The sequence corresponding to the protein cleaved from the SUMO expression tag is shown in bold.
^{b}The optimized Cat^{C} intein construct with appended charged residues utilized for the structural studies
^{c}The WT intein sequences are shown for both MBP-Cat^{N} and Cat^{C}-GFP. The underlined residues correspond to the positions of mutation for the extein activity screen.

### Expression and Purification of Inteins for Splicing Assay

Expression and purification of the inteins was carried out as previously described. The expressed N-intein constructs contained the following architecture: His₆-SUMO-MBP-EFE-Int^{N}, where “His₆” is a 6× polyhistidine affinity tag, “SUMO” is the ubiquitin-like protein SMT3, “MBP” is maltose binding protein, “EFE” is the wild type-1, -2, and -3 N-extein sequence of TerL inteins, and Int^{N} is the N-intein. The expressed C-intein constructs contained the following architecture: His6-SUMO-Inti-CEFL-GFP, where “Int^{C}” is the C-intein, “CEFL” is the +1, +2, +3, and +4 C-extein residues of TerL inteins, and “GFP” is green fluorescent protein. For the screen of extein dependence, constructs corresponding to each indicated point mutation in the “EFE” or “CEFL” extein sequences were utilized.

E. coli BL21(DE3) cells were transformed with an MBP-Int^{N} or Int^{C}-GFP intein plasmid and grown at 37° C. in 1 L of LB containing 50 pg/mL of kanamycin. Once the culture reached an OD₆₀₀=0.6, 0.5 mM IPTG was added to induce expression (0.5 mM final concentration, 18 h at 18° C.). For test expression of the SUMO-Cats constructs, expression tests were also carried out at 37° C. for 3 hours upon addition of IPTG. Following expression, the cells were pelleted via centrifugation (5,000 rcf, 30 min) and stored at −80° C.

The cell pellet was then resuspended in 30 mL of lysis buffer (50 mM phosphate, 300 mM NaCl, 5 mM imidazole, pH 8.0) containing a protease inhibitor cocktail. The cells were lysed by sonication (35% amplitude, 8×20 s pulses on/30 s off) and then pelleted by centrifugation (35,000 rcf, 30 min). The supernatant was incubated with 4 mL of Ni-NTA resin for 30 min at 4° C. to bind the His-tagged inteins. The slurry was then loaded onto a fritted column, the flow through was collected, and the column was washed with 20 mL of lysis buffer. The protein was then eluted from the column with 20 mL of elution buffer (lysis buffer+250 mM imidazole).

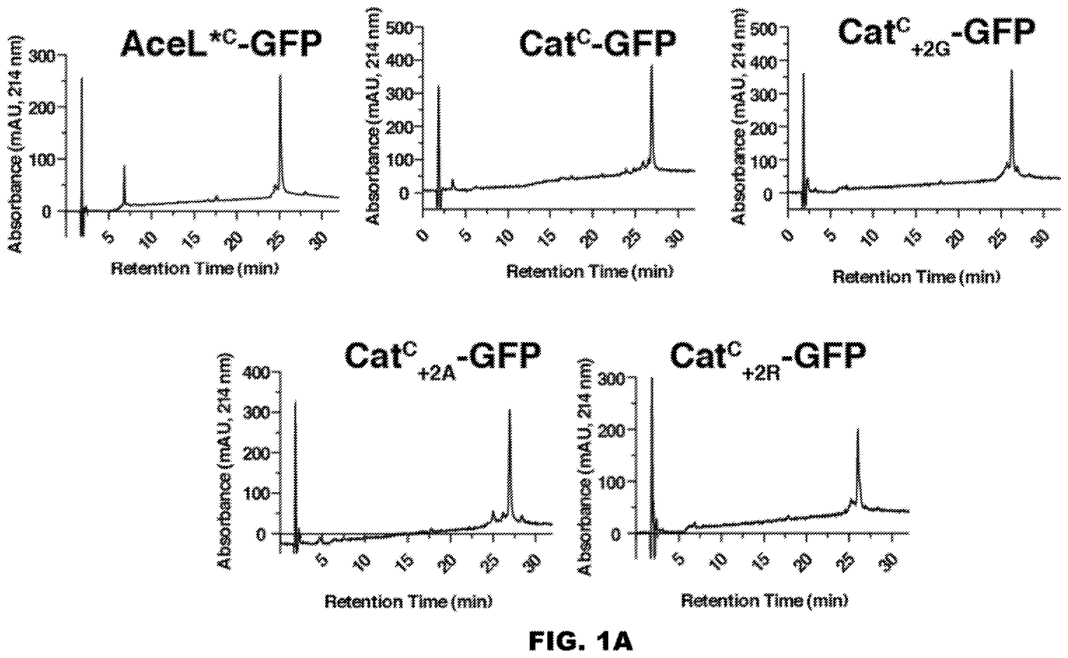

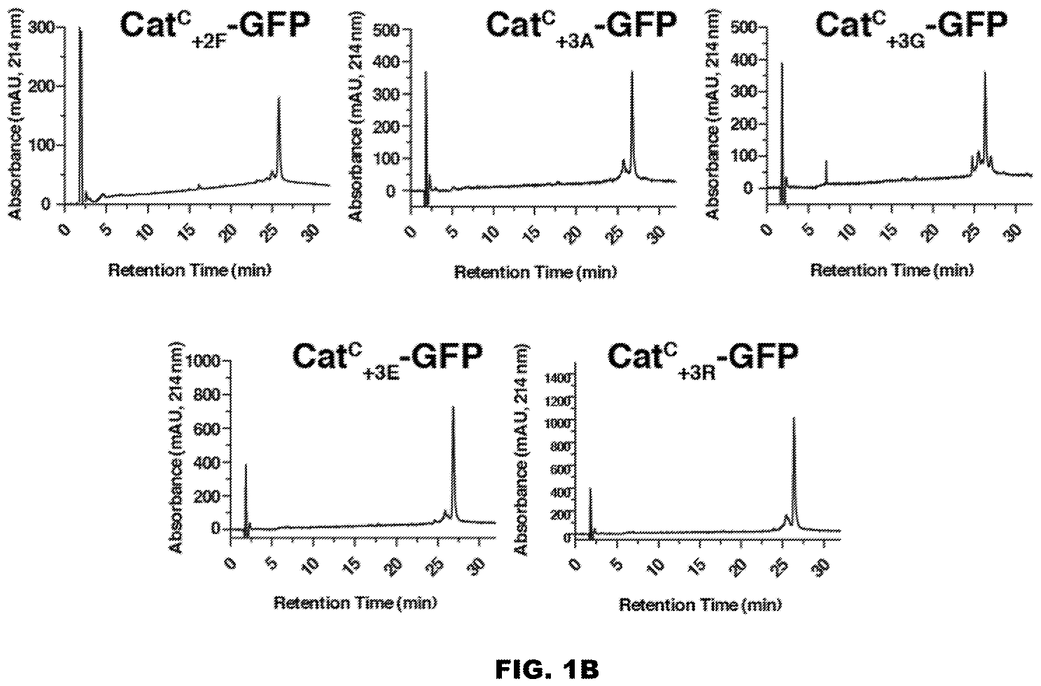

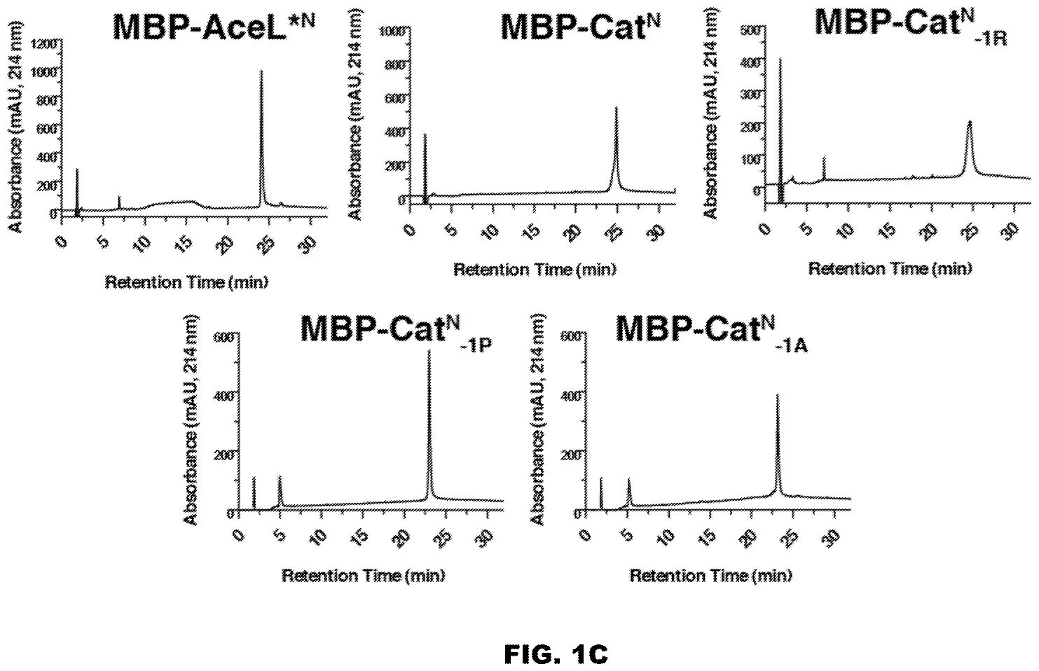

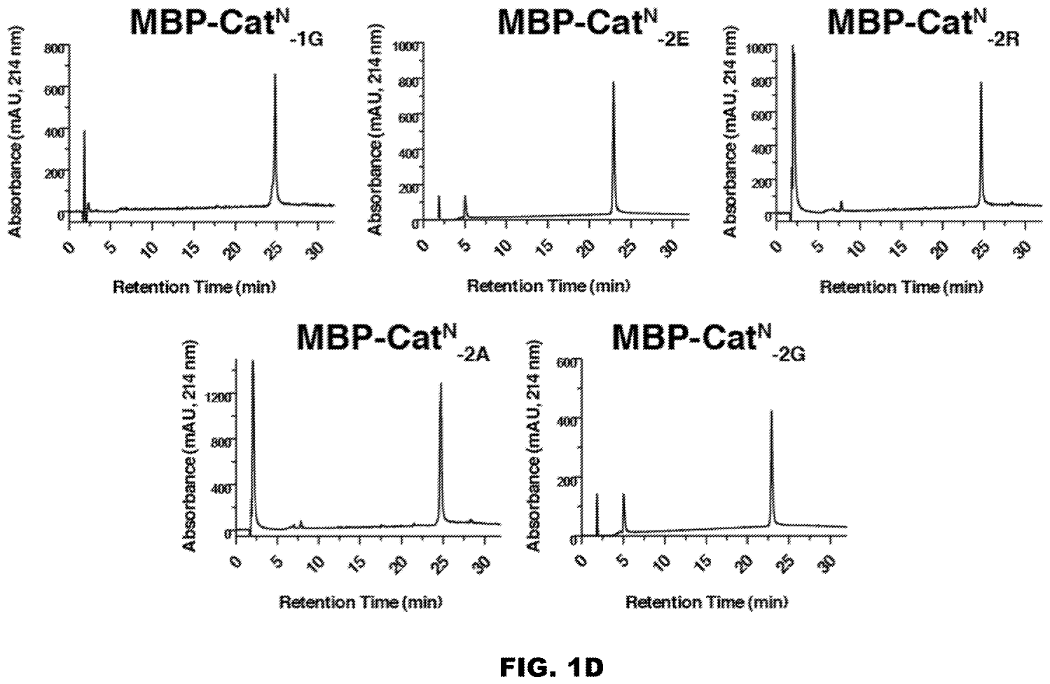

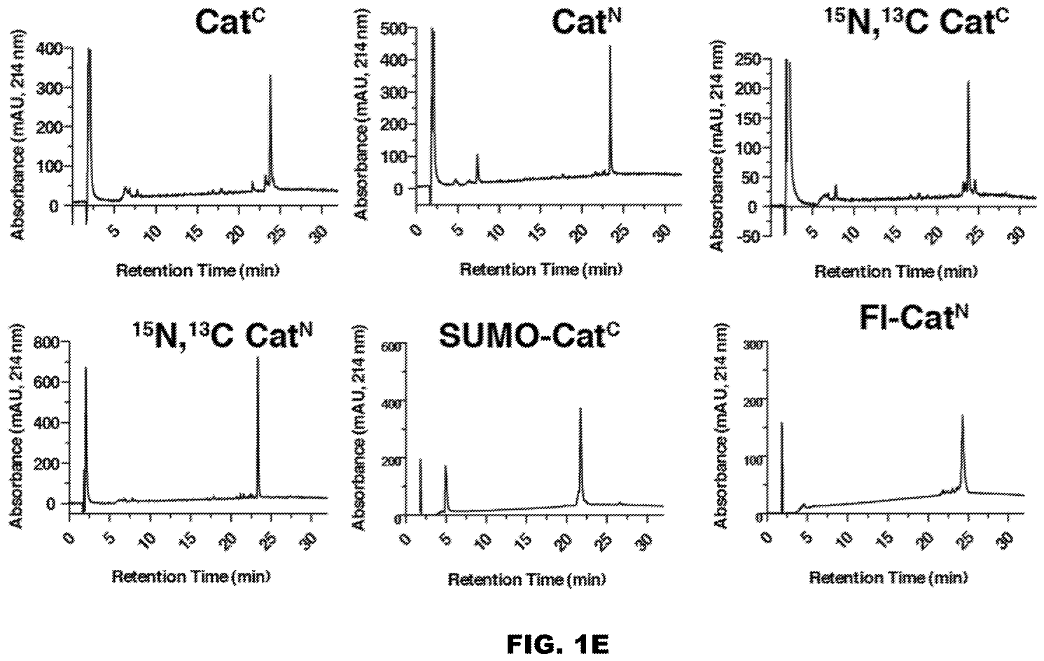

The eluted protein was dialyzed into lysis buffer while being treated with 10 mM TCEP and Ulp1 protease overnight at 4° C. to cleave the His₆-SUMO expression tag. The dialyzed protein was then incubated with 4 mL Ni-NTA resin for 30 min at 4° C., after which it was applied to a fritted column with the flow through collected together with a 10 mL wash of lysis buffer. The protein was then treated with 10 mM TCEP, concentrated to 2 mL, and purified over an S75 16/60 gel filtration column using degassed splicing buffer (100 mM sodium phosphate, 150 mM NaCl, 1 mM EDTA, pH 7.2) as the mobile phase. Fractions were analyzed by analytical RP-HPLC and ESI-MS (FIG. 1, Table 3), and either immediately utilized in the splicing assay or stored long term in glycerol (20% v/v) after being flash-frozen in liquid N₂.

TABLE 3
Masses of purified proteins.

| eGFP Proteins | Expected Mass (Da) | Observed Mass (Da) |
|---|---|---|
| AceL*^{C}-GFP | 40424.6 | 40424.3 |
| Cat^{C}-GFP | 40277.7 | 40275.6 |
| Cat^{C}_{+2G}-GFP | 40205.5 | 40205.8 |
| Cat^{C}_{+2A}-GFP | 40219.5 | 40218.7 |
| Cat^{C}_{+2R}-GFP | 40304.6 | 40305.8 |
| Cat^{C}_{+2F}-GFP | 40295.6 | 40294.9 |
| Cat^{C}_{+3R}-GFP | 40286.6 | 40286.4 |
| Cat^{C}_{+3A}-GFP | 40201.5 | 40201.0 |
| Cat^{C}_{+3G}-GFP | 40187.5 | 40187.3 |
| Cat^{C}_{+3E}-GFP | 40259.5 | 40258.4 |
| MBP Proteins | Expected Mass (Da) | Observed Mass (Da) |
| MBP-Cat^{N} | 44094.0 | 44093.4 |
| MBP-AceL*^{N} | 43508.3 | 43508.1 |
| MBP-Cat^{N}_{-1R} | 44121.1 | 44120.2 |
| MBP-Cat^{N}_{-1A} | 43036.0 | 44034.5 |
| MBP-Cat^{N}_{-1G} | 44022.0 | 44022.4 |
| MBP-Cat^{N}_{-1P} | 44062.0 | 44062.8 |
| MBP-Cat^{N}_{-2R} | 44103.0 | 44101.9 |
| MBP-Cat^{N}_{-2A} | 44017.9 | 44017.2 |
| MBP-Cat^{N}_{-2E} | 44076.0 | 44076.4 |
| MBP-Cat^{N}_{-2G} | 44003.9 | 44002.8 |
| Proteins for NMR | Expected Mass (Da) | Observed Mass (Da) |
| FLAG-Cat^{C} | 13499.2 | 13499.9 |
| Cat^{N} | 3773.2 | 3773.0 |
| ¹⁵N, ¹³C FLAG-Cat^{C} | 14257.6 | 14247.5 |
| ¹⁵N, ¹³C Cat^{N} | 3974.7 | 3972.7 |
| Proteins for Binding | Expected Mass (Da) | Observed Mass (Da) |
| Fl-Cat^{N} | 4187.6 | 4186.7 |
| SUMO-Flag-Cat^{C} | 26766.0 | 26764.7 |

### Splicing Assays

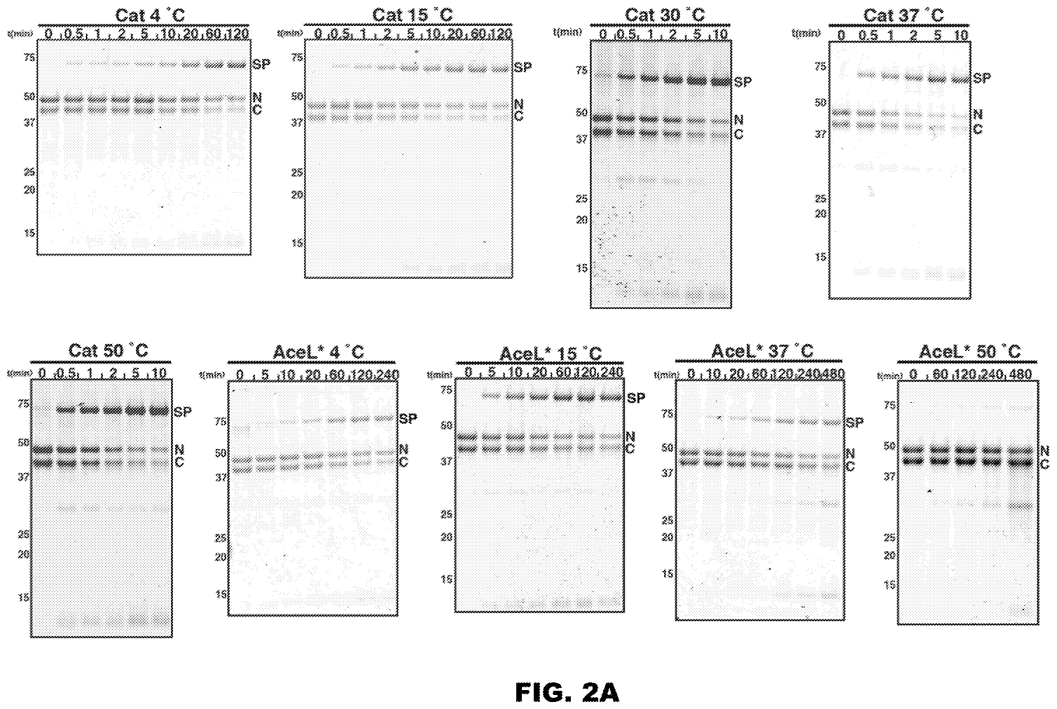

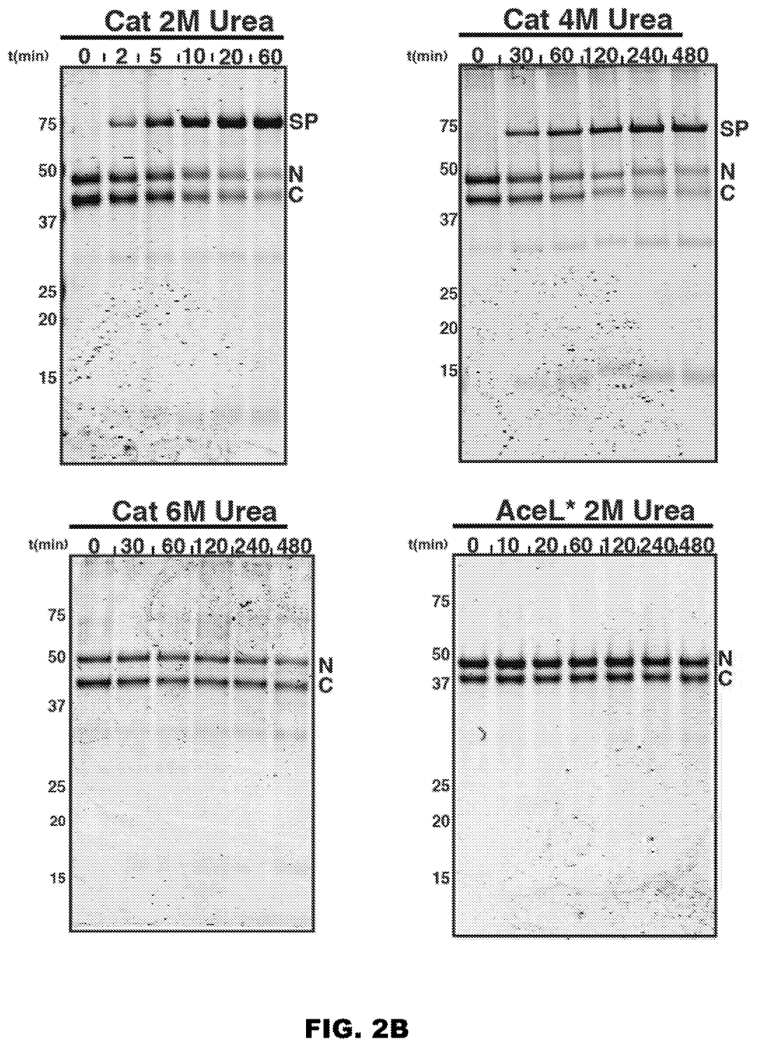

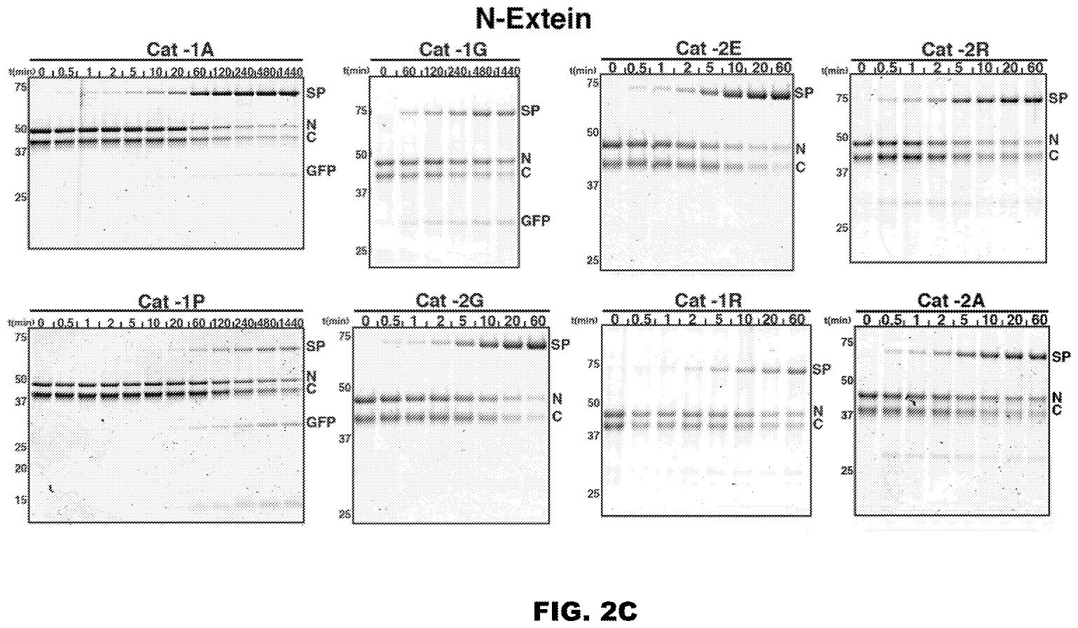

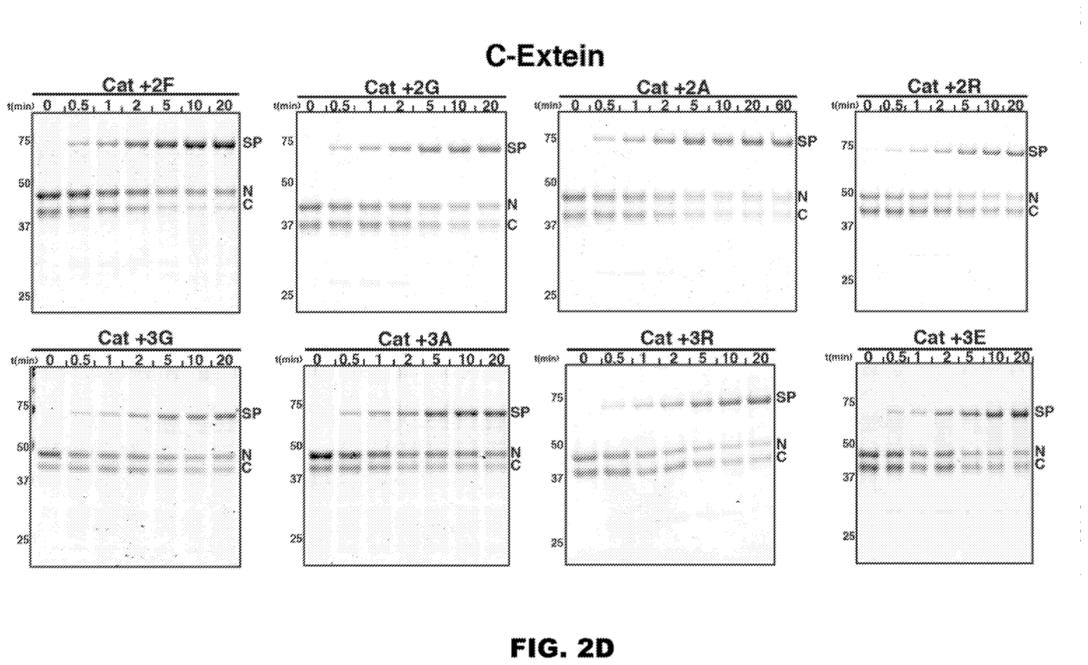

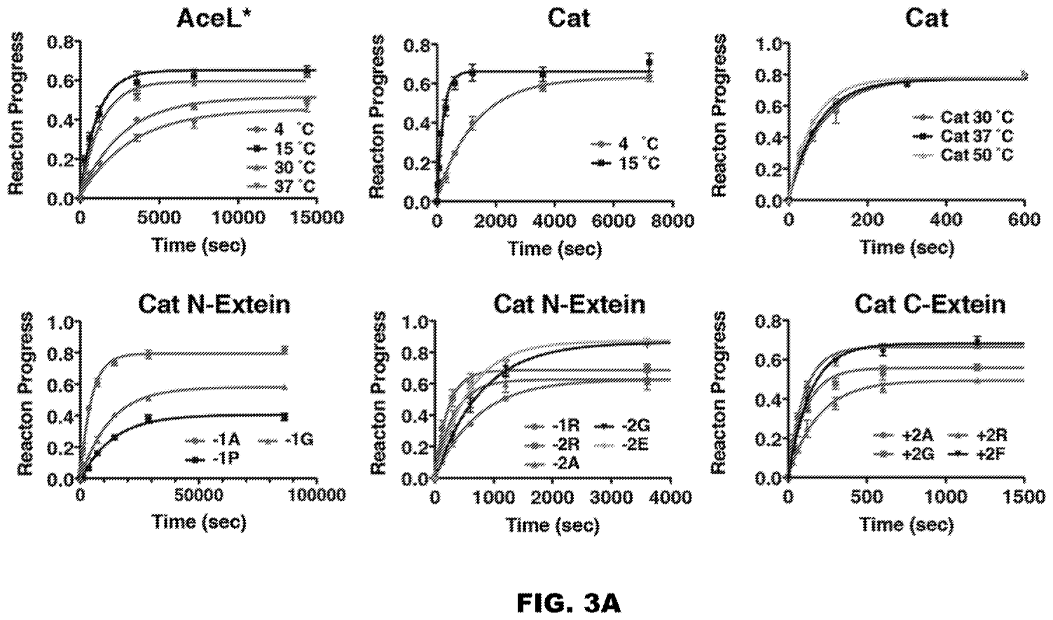

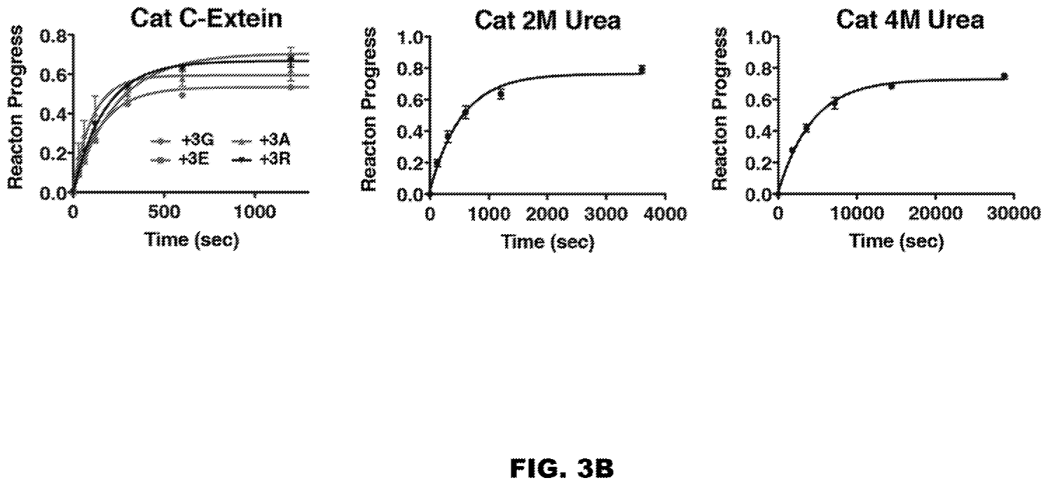

Splicing assays were carried out as adapted from a previously described protocol.⁸ Briefly, N- and C-inteins (4 pM Int^{N}, 4 μM Int^{C}) were individually preincubated in splicing buffer (100 mM sodium phosphates, 150 mM NaCl, 1 mM EDTA, pH 7.2) with 2 mM TCEP for 15 min. Splicing reactions were carried out at indicated temperatures and concentrations of urea. For the extein characterization, the Cat^{C}-GFP and MBP-Cat^{N} proteins containing the indicating extein mutations were spliced with their cognate wild type N- or C-intein at 30° C. Splicing of Cat and AceL* in the presence of urea was carried out at 30° C. Splicing was initiated by mixing equal volumes of N- and C-inteins with aliquots removed at the indicated times and quenched by the 1:1 addition of 4× loading dye (160 mM Tris, 40% glycerol, 4% SDS, 0.08% Bromophenol Blue, 8 BME). Samples were analyzed by SDS-PAGE gel electrophoresis (12% bis-tris, 60 min, 150 v) and quantified by densitometry (FIGS. 2 and 3).

### Kinetic Analysis of Trans-Splicing Reactions

To determine the splicing rates of trans-splicing reactions, the data was fit to the first order rate equation using GraphPad Prism software.

[P](t)=[P]ₘₐₓ(1−e^{-kt})

Where [P] is the normalized intensity of product, [P]ₘₐₓ is the reaction plateau, and k is the rate constant (s⁻¹). The mean and standard error for each value are reported (n=3).

### Expression of Inteins for Structural Studies

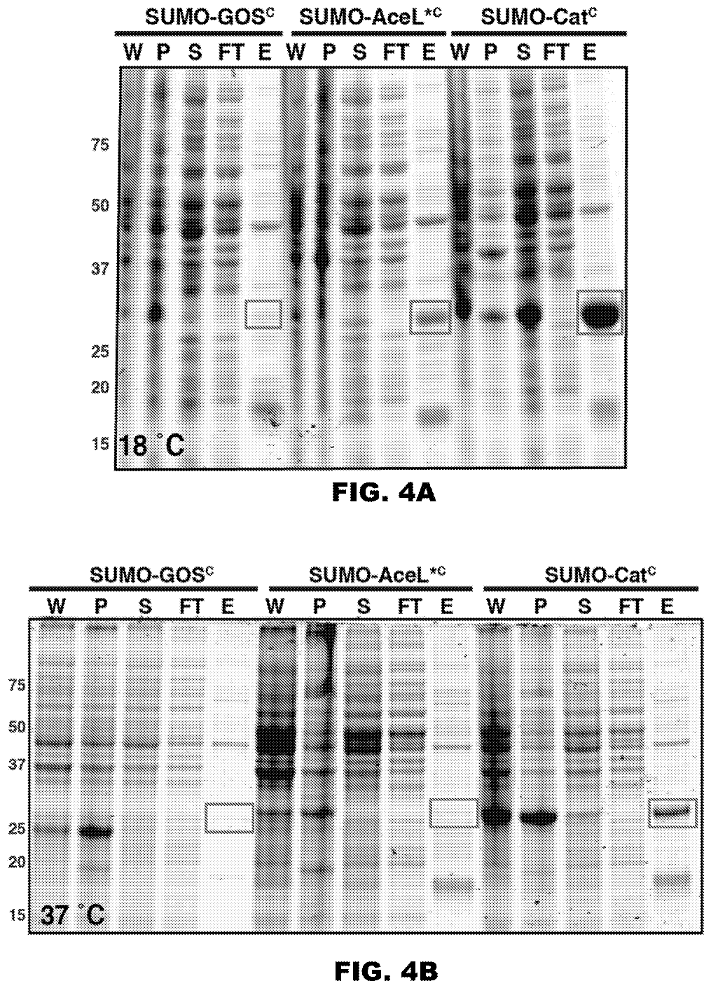

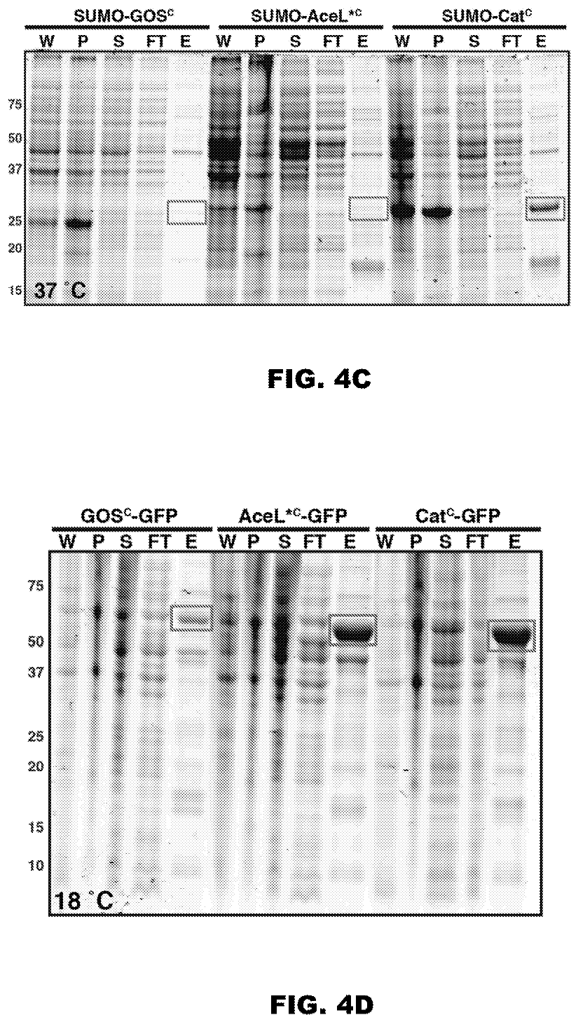

Construct optimization was required in order to isolate Cat^{C} with minimal extein sequence for structural characterization. Compared to AceL*^{C} and GOS^{C}, SUMO-Cat^{C} had increased yields during recombinant expression in E. coli (18° C., 16 h or 37° C. for 3 h) (FIG. 4). However, removal of the SUMO expression tag resulted in Cat^{C} aggregating upon cleavage (possibly due its neutral charge at physiological pH, pI=7.2). Charged residues were therefore appended immediately flanking Cat^{C} to improve the solubility of the protein in solution, specifically an N-terminal FLAG epitope tag and “CESRGK” C-extein sequence (SUMO-Flag-Cat^{C}). The Cat^{N} construct utilized in these structural studies was expressed as a SUMO fusion (SUMO-Cat^{N}) and contains the minimal “EFE” N-extein following SUMO cleavage. In addition, inactivating C1A and N134A mutations were included in the constructs to prevent splicing during structural analysis of the associated complex. Expression and purification of these Cat^{N} and Cat^{C} constructs for structural study were carried out as described above for the proteins utilized for splicing.

For use in NMR spectroscopy, expression of the isotopically enriched Cat proteins was carried out as previously described. The intein plasmids were used to transform BL-21 (DE3) cells, and the cells were grown overnight in 5 mL LB starter cultures (37° C., 18 h). The starter cultures were then spun down (4,000 rcf, 5 min). The supernatant was discarded, and the cells were then resuspended and grown in 1 L of M9 medium supplemented with ¹³C-glucose and ¹⁵NH₄Cl as the sole carbon and nitrogen sources (50 μg/mL kanamycin, 37° C.). Once the cells reached OD₆₀₀=0.6, expression was induced with the addition of IPTG (0.5 mM, 18 h, 18° C.). Following expression, the cells were spun down by centrifugation (5,000 rcf, 30 min) and stored at −80° C. Purification was carried out with the general method described above for intein constructs. The masses of the purified proteins correspond to an isotopic labeling efficiency of 99% for both the Cat^{N} and Cat^{C} proteins.

### NMR Spectroscopy

NMR experiments were performed using Cat^{N} and Cat^{C} in free form and in complex. NMR samples were prepared by buffer exchanging purified protein to 20 mM sodium phosphate 150 mM NaCl, 2 mM TCEP (pH 6.8, 37° C.). The uniformly labeled ¹⁵N, ¹³C, ¹H proteins were concentrated to final concentrations of ˜300-600 pM. For the HSQC experiments of the complex reported in FIGS. 3A, 3B, the isotopically labeled intein fragments were mixed with the complementary unlabeled intein solution in a ratio of 1:1.5 and concentrated to a final concentration similar to the free protein and measured directly. For structure determination isotopically labeled intein fragments were mixed at a Cat^{N}:Cat^{C} ratio of 1.5:1. The complex was further purified by size exclusion chromatography to remove the free forms.

Experiments were performed at field strengths of 600, 700, 800 or 900 MHz and Non-Uniform Sampling (NUS) acquisition was employed as appropriate. NMR spectra were processed using Bruker Topspin 3.0 or NMR Pipe software and NUS spectra were reconstructed by compressed sensing using qMDD.

### Chemical Shift Assignment

Backbone chemical shifts were assigned using HNCO, HN(CA)CO, HNCACB, CBCA(CO)NH triple resonance experiments. Side chain assignments were obtained from H(CC)(CO)NH, (H)CC(CO)NH, H(C)CH-TOCY and (H)CCH-TOCSY experiments. Aromatic assignments were obtained from CT-¹³C-resolved [¹H,¹H]-NOESY (mixing time=100 ms), (HB)CB(CGCD)HD and (HB)CB(CGCDCE)HE experiments. CcpNmr Analysis software was used for manual chemical shift assignment and other data analysis Chemical shift values have been validated and deposited to the Biological Magnetic Resonance Bank (BMRB No: 30480). Random coil chemical shifts were calculated using CcpNmr analysis.

### Spin Relaxation Measurements

Spin-spin relaxation (R₂) rates of ¹⁵N spins (mixing times of 0, 17, 34, 51, 85, 119, 170, 255, 340, 510, 680 ms) and [¹⁵N-¹H] NOE experiments were measured at a field strength of 600 MHz.

### Structure Determination

Dihedral angle restrains were calculated from chemical shifts using TALOS software.¹³ NOE cross peaks were picked from ¹⁵N-resolved [¹H,¹H]-NOESY (mixing time=80 ms), ¹³C-resolved-[¹H,¹H]-NOESY (mixing time=80 ms), CT-¹³C-resolved aromatic [¹H,¹H]-NOESY experiments (mixing time=100 ms) and assigned automatically using ARIA and CNS softwares. Assignment and structure calculation was done in 8 cycles, calculating 20 structures in each step. The assigned NOEs were verified manually and violation analysis was done. The verified NOE peak lists were used to generate distance restraints. 3,283 unambiguous restraints, 206 ambiguous restraints and 180 dihedral angle restraints were used to finally calculate 256 structures. 20 least energy structures were selected and water refinement was performed. Structures have been validated and deposited to the Protein Data Bank (PDB ID: 6DSL).

### Circular Dichroism (CD)

Cat^{N}, Cat^{C}, and 1:1 complex of Cat^{N} and Cat^{C} were dialyzed into CD buffer (25 mM sodium phosphate, 50 mM NaF, 1 mM DTT, pH 7.2). CD spectra were measured at 25° C. in a 1 mm pathlength cuvette (10 pM sample concentration).

### Analytical Size Exclusion Chromatography (SEC)

Analytical SEC experiments were run on an S75 10/300 column at 4° C. in splicing buffer (25 mM sodium phosphate, 150 mM NaCl, 1 mM DTT, pH 7.2. For all runs, UV absorbance was monitored at 214 nm. Samples were injected with a sample volume of 500 μL (25 pM) and eluted with a flow rate of 0.5 mL/min.

### Limited Proteolysis

EFE-Cat^{N}, Flag-Cat^{C}, and 1:1 complex of EFE-Cat^{N} and Flag-Cat^{C} were dialyzed into thermolysin buffer (50 mM Tris HCl, 100 mM NaCl, 2 mM MgSO4, 2 mM CaCl2, 1 mM DTT, pH 7.4) and diluted to a concentration of 10 μM. Thermolysin powder (Sigma) dissolved to 0.4 mg/mL in thermolysin buffer was then prepared and added to each solution (1:50 v/v). At the indicated times, aliquots were removed and quenched with the 1:3 addition of 8 M Guanidine HCL 4% TFA. The samples were then analyzed by RP-HPLC and ESI-MS. Masses from each peak were compared to predicted cleavage products of the inteins from ProteinProspector (UCSF).

### Production of Inteins for Binding Experiments

The fluorescein labeled Cat^{N} (FI-Cat^{N}) peptide was synthesized by standard 9-fluorenylmethyl-oxycarbonyl (Fmoc) solid phase peptide synthesis (SPPS). After coupling the last amino acid in the peptide, the N-terminus was capped with 5(6)-carboxyfluorescein. The synthesized FI-Cat^{N} peptide was purified by preparative RP-HPLC and characterized by analytical RP-HPLC and ESI-MS. The C-intein expressed for the binding experiments was SUMO-Flag-Cats construct detailed above. Instead of carrying out an Ulp1 digestion, the expressed SUMO-Flag-Cats protein was purified directly over the S75 16/60 gel filtration column following Ni-NTA enrichment.

### Steady State Fluorescence Anisotropy

Equilibrium measurements were performed using 500 pM Fl-Cat^{N} with given concentrations of SUMO-Flag-Cats (0 pM-2,500 pM) in low salt (50 mM sodium phosphate, 100 mM NaCl, 1 mM DTT, 1 mM EDTA, pH 7.0) and high salt (50 mM sodium phosphate, 500 mM NaCl, 1 mM DTT, 1 mM EDTA, pH 7.0) buffers. Proteins were diluted from stock solutions to desired concentrations and incubated at 25° C. for 30 min. Samples were transferred to a cuvette of 1 cm path-length and the fluorescence anisotropy was measured immediately. Constants in the one site binding equation were obtained using non-linear least squares curve fitting method in MATLAB. For both the high and low salt conditions, the constants obtained from these fits (Table 4) fall below the concentration of Cat^{N} used for the measurements. We therefore report the K_{d} as <500 pM, as we were unable to measure fluorescence anisotropy at lower concentrations of Cat^{N}.

TABLE 4
Kinetic binding constants.

| | 100 mM NaCl | | 500 mM NaCl | |
|---|---|---|---|---|
| [Cat^{C}] | | | | |
| (nM) | k_{obs1} (s⁻¹) | k_{obs2} (s⁻¹) | k_{obs1} (s⁻¹) | k_{obs2} (s⁻¹) |
| 200 | 0.60 ± .07 | 0.08 ± 0.09 | 0.44 ± 0.04 | 0.05 ± 0.009 |
| 325 | 0.78 ± .08 | 0.07 ± 0.06 | 0.92 ± 0.12 | 0.09 ± 0.009 |
| 500 | 1.10 ± .12 | 0.10 ± 0.015 | 0.90 ± 0.08 | 0.10 ± 0.011 |
| 750 | 2.08 ± .16 | 0.15 ± 0.014 | 1.87 ± 0.22 | 0.16 ± 0.013 |
| 1000 | 2.74 ± .18 | 0.20 ± 0.010 | 2.32 ± 0.22 | 0.20 ± 0.020 |
| | kₒₙ₁ (M⁻¹ s⁻¹) | kₒₙ₂ (M⁻¹ s⁻¹) | kₒₙ₁ (M⁻¹ s⁻¹) | kₒₙ₂ (M⁻¹ s⁻¹) |
| Fit | (2.80 ± | (0.16 ± | (2.34 ± | (0.18 ± |
| | .28) × 10⁶ | .019) × 10⁶ | 0.30) × 10⁶ | 0.016) × 10⁶ |

### Stopped Flow Fluorescence Anisotropy

The stopped flow syringes were loaded with FI-Cat^{N} and SUMO-Flag-Cat^{C} protein solutions so as to obtain final concentrations of 100 nM Cat^{N} and reported concentrations of Cat^{C} (200, 325, 500, 750, 1000 nM). Change in anisotropy values were measured in low salt and high salt buffers for a duration of 50 s. The change in anisotropy over time was fit to a double exponential kinetic model previously reported using non-linear least squares curve fitting method in MATLAB to obtain kinetic constants of binding (k_{obs1} and k_{obs2}) for each concentration.¹⁶ The k_{obs1} and k_{obs2} values were then plotted as a function of Cat^{C} concentration, fit to a line, and the slope of the line was interpreted as kₒₙ.

### Results

1. Design of a Consensus Atypical Split Intein with Enhanced Stability and Activity

In order to determine the mechanism of fragment association, an atypically split intein with minimal extein residues was isolated. Both naturally occurring atypical split inteins whose splicing rates have been characterized in vitro were identified within the T4-bacteriophage-type DNA-packaging terminase large subunit (TerL) from metagenomic sequencing data. The first, from the saline meromictic Ace Lake in Antarctica (AceL), exhibits an optimal splicing rate at 8° C. (t1/2=7 min). In addition, directed evolution found stabilizing mutations within AceL (AceL*) that increase activity at 37° C. (t1/2=6 min). The second characterized atypical split intein was sequenced in a sample collected from Punta Cormorant in the global ocean sampling project (GOS) and splices at an optimal temperature of 30° C. (t1/2=3 min). Purification of soluble GOS^{N} (i.e. the N-terminal GOS intein fragment), GOS^{C}, or AceL*^{C} from expression in E. coli was performed by means of large stabilizing extein proteins (FIG. 4). The extraction of atypically split inteins lacking solubilizing exteins from the insoluble inclusion body fraction with chaotropic agents was unsuccessful due to aggregation issues while refolding.

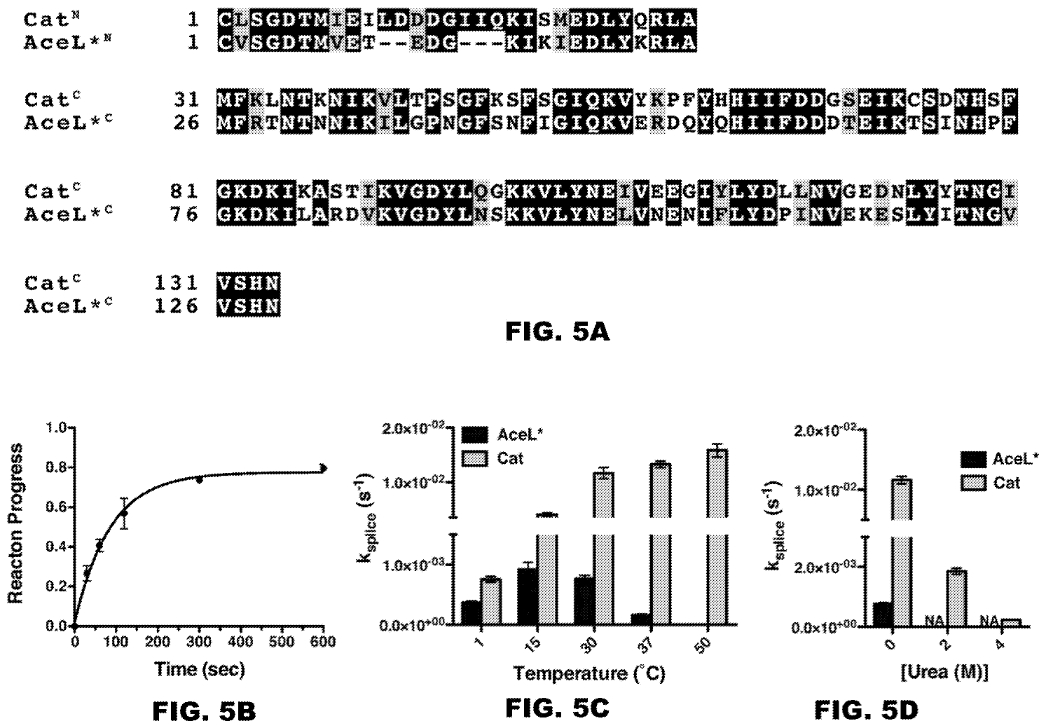

Consensus design is a protein engineering strategy that utilizes evolutionary information from homologous protein sequences to predict stabilizing mutations and has previously been applied to generate a highly active and thermostable naturally split DnaE intein (Cfa). Seeking to engineer an atypically split intein amenable to in vitro structural characterization, a consensus atypical (Cat) TerL intein from multiple sequence alignments (MSA) of TerL^{N} and TerL^{C} inteins discovered from BLAST searches of metagenomic sequencing information in the JGI and NCBI databases was designed (Table 1). Both Cat^{N} (60%) and Cat^{C} (64%) contain high sequence similarity to AceL*^{N} and AceL*^{C} respectively, with the nonidentical residues spread throughout the primary sequence (FIG. 5). The Cat intein pair was isolated fused to model exteins to measure its in vitro trans-splicing activity (Table 5). Cat exhibits ultrafast splicing activity (t_{1/2}=59 s at 30° C.) and consistently outperforms AceL* across an array of temperatures (FIG. 5). Moreover, Cat remains active at 50° C., a temperature at which AceL* fails to splice. PTS was also measured in the presence of chaotropic agents, which are often utilized to solubilize aggregation-prone extein fragments. 1 Cat displays enhanced chaotropic stability and can splice in both 2 M and 4 M urea (FIG. 5, Table 6), while AceL* is inactive under both of these conditions. The accelerated splicing rates and activity under adverse conditions establish Cat as the fastest and most robust atypical split intein reported to date, and it should therefore serve as a tool for the synthetic N-terminal modification of proteins.

TABLE 5
Protein Splicing at Indicated Temperatures.

| Intein | Temp (° C.) | k_{splice} (s⁻¹) | t_{1/2} (s) |
|---|---|---|---|
| AceL* | 4 | (3.70 ± .26) × 10⁻⁴ | 1873 ± 132 |
| AceL* | 15 | (9.17 ± 1.2) × 10⁻⁴ | 756 ± 100 |
| AceL* | 30 | (7.68 ± .58) × 10⁻⁴ | 902 ± 68 |
| AceL* | 37 | (3.03 ± .30) × 10⁻⁴ | 2287 ± 228 |
| Cat | 4 | (7.54 ± .48) × 10⁻⁴ | 919 ± 58 |
| Cat | 15 | (4.81 ± .48) × 10⁻³ | 144 ± 14 |
| Cat | 30 | (1.17 ± .10) × 10⁻² | 59 ± 5 |
| Cat | 37 | (1.32 ± .06) × 10⁻² | 52 ± 2 |
| Cat | 50 | (1.58 ± .12) × 10⁻² | 44 ± 3 |

TABLE 6
Protein Splicing in Chaotropic Agents.

| Intein | [Urea] (M) | k_{splice} (s⁻¹) | t_{1/2} (s) |
|---|---|---|---|
| Cat | 2 | (1.86 ± .18) × 10⁻³ | 373 ± 35 |
| Cat | 4 | (1.73 ± 1.7) × 10⁻⁴ | 3826 ± 368 |

2. Fragment Assembly Drives a Disorder to Order Structural Transition

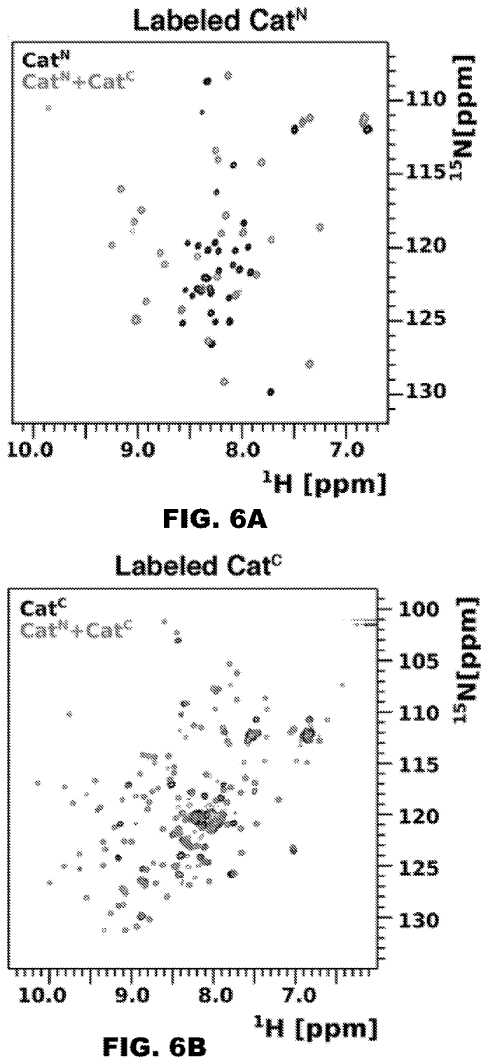

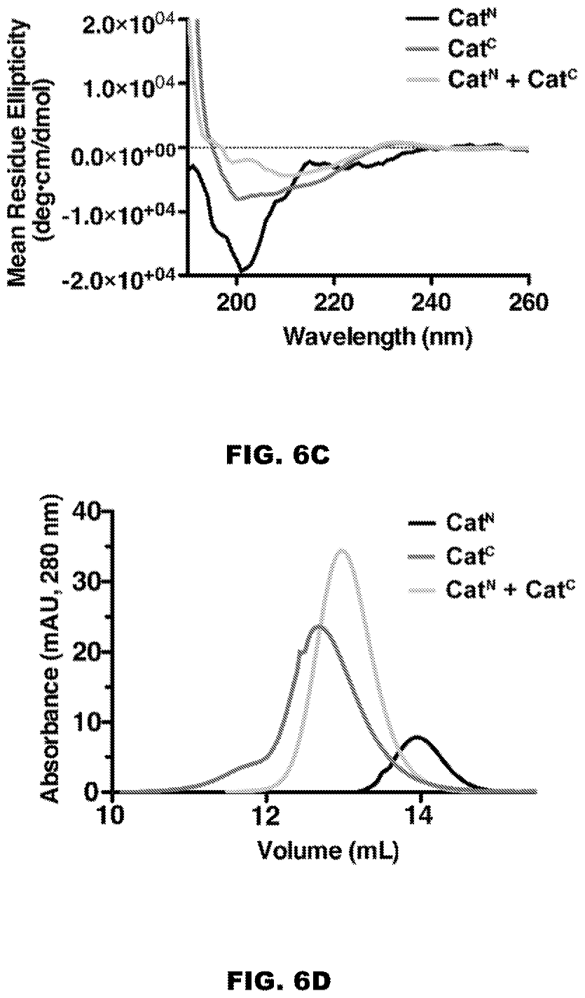

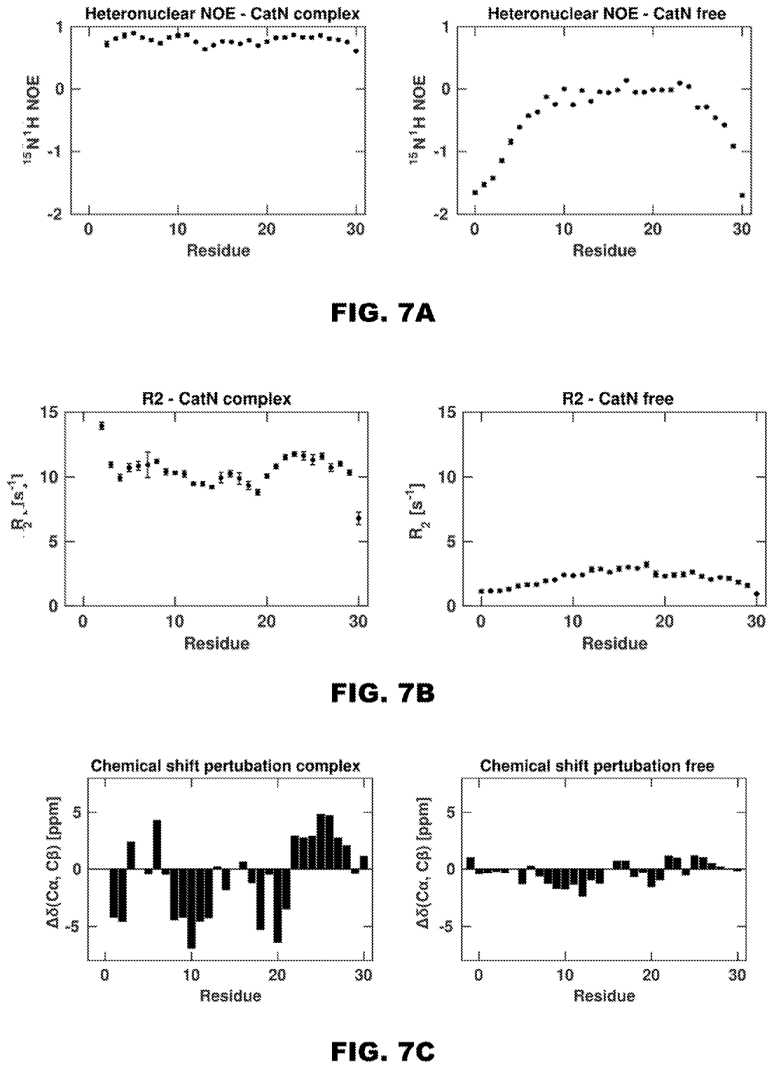

To investigate the association process of atypical split inteins, Cat^{N} and Cat^{C} bearing minimal exteins were expressed in isotopically enriched media (¹⁵N, ¹³C), purified, and analyzed by nuclear magnetic resonance (NMR) spectroscopy. Note, these constructs also included inactivating C1A and N134A mutations to prevent splicing during structural analysis of the complex. The ¹H-¹⁵N HSQC spectrum of Cat^{N} in isolation displays minimal dispersion along the ¹H dimension, a common phenomenon among disordered proteins and previously observed for Ssp^{C} and Npu^{C} (FIG. 6). A stark transition occurs upon addition of unlabeled Cat^{C}, resulting in a well dispersed ¹H-¹⁵N HSQC spectrum, which is consistent with Cat^{N} folding (FIG. 6). Furthermore, measurements of ¹H-¹⁵N heteronuclear NOEs, spin-spin relaxation rates, and Cα-Cβ chemical shift perturbation in Cat^{N} provide additional evidence for a disorder to order transition in Cat^{N} upon binding Cat^{C} (FIG. 7). The ¹H-¹⁵N HSQC of Cat^{C} in isolation exhibits far fewer crosspeaks than expected from the number of residues in the protein, a feature present in dynamic proteins that are undergoing chemical exchange and previously observed in both SspN and NpuN (FIG. 6). Addition of unlabeled Cat^{N} leads to the appearance of new crosspeaks, which indicates a transition to a more ordered complex (FIG. 6). Although the spectral quality of Cat^{C} in free form precluded our ability to assign the protein, some crosspeaks overlap those observed in the bound form, which suggests that Cat^{C} in free and bound form share a partial structural identity.

In line with the NMR studies, analysis by circular dichroism spectroscopy indicates that unbound Cat^{N} is largely unstructured with some propensity to sample secondary structure, and that both Cat^{N} and Cat^{C} inteins undergo a structural transition upon association (FIG. 6). Further evidence for folding upon binding was observed by size exclusion chromatography (SEC), as Cat^{C} elutes at an earlier time than the bound complex despite having a lower molecular weight (FIG. 6). The SEC elution profile is consistent with a compaction of Cat^{C} upon binding its cognate intein.

3. Solution Structure of an Atypical Split Intein Complex

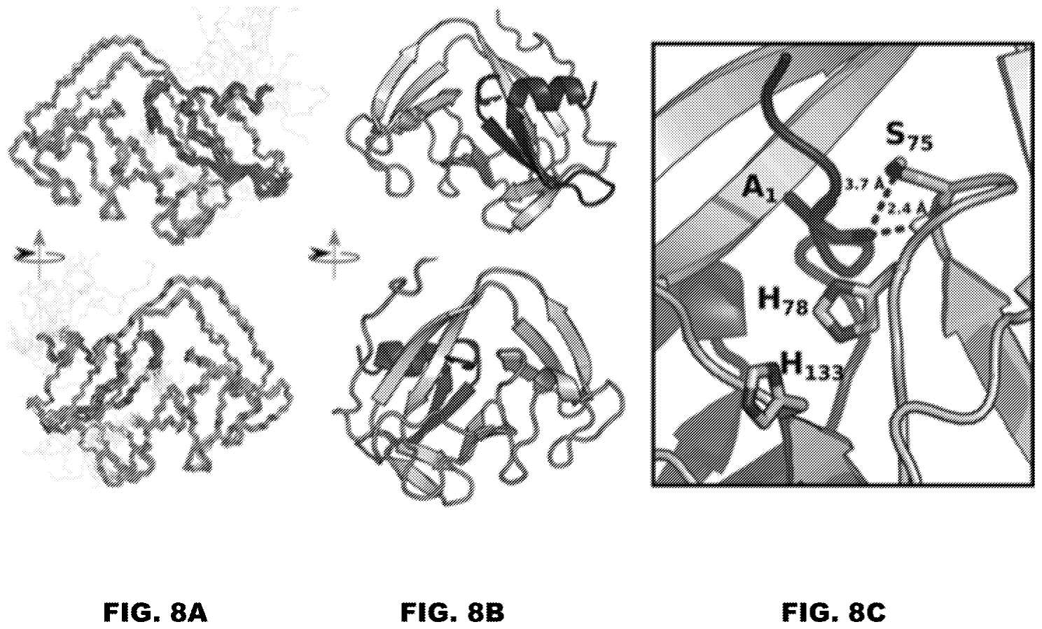

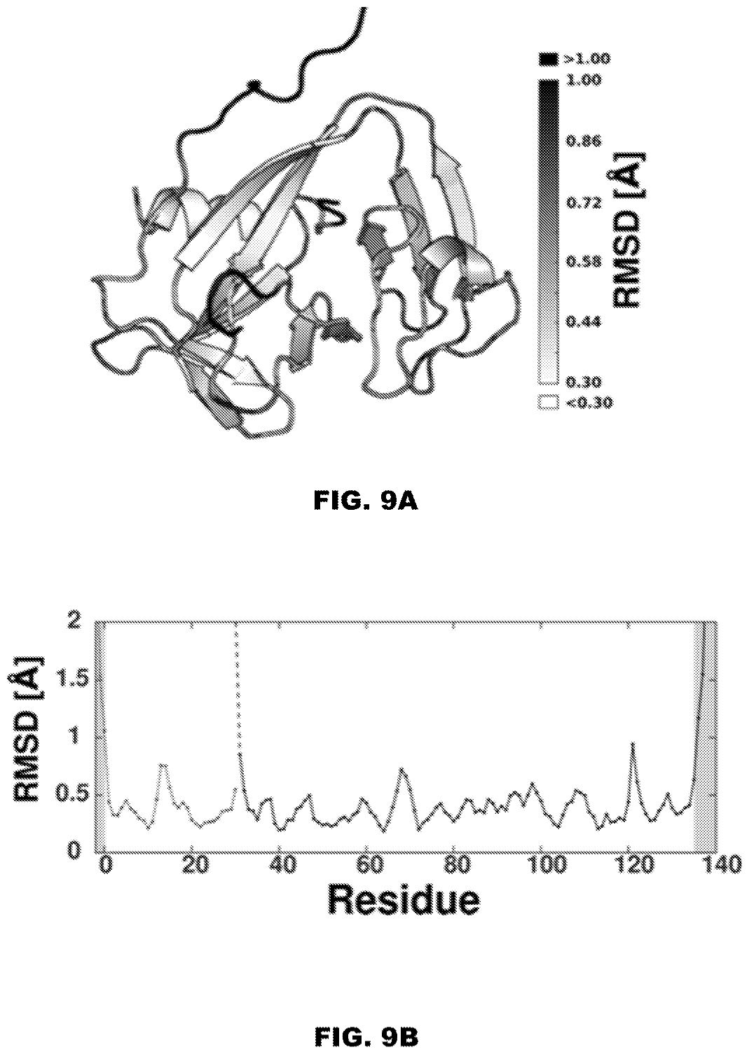

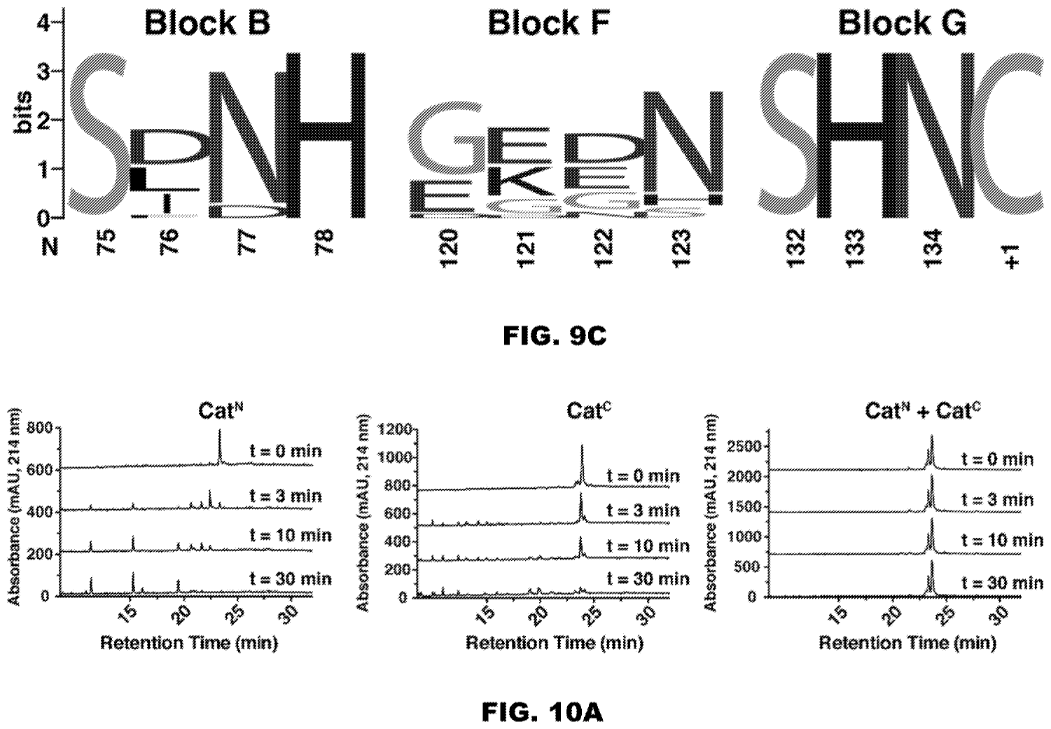

The isotopically enriched Cat^{N} and Cat^{C} proteins were assembled into a complex, and its structure was calculated from distance restraints and dihedral angle constraints obtained from NMR spectroscopy. The twenty lowest energy conformers obtained from the structure calculation are shown (FIG. 8A, PDB ID: 6DSL). The structure ensemble is precise in all regions of the protein (with the exception of a short solubility tag in Cat^{C} and the exteins) with a mean backbone RMSD of 1.19 Å to the average structure (Table 7). Residue wise backbone RMSD values of <0.5 Å were obtained across the structured regions of the protein (FIGS. 9A and 9B). The structure of Cat is predominantly β-sheet, with the last 8 residues present in the C-terminus of Cat^{N} being the only α-helix (FIG. 8). It has a horseshoe-like shaped structure that is typical for proteins containing the HINT domain. The structure of Cat is similar to that of DnaE inteins, such as Npu (PDB ID: 2KEQ, RMSD 1.45 Å over 92 aligned Ca atoms) and Ssp (PDB ID: 1ZDE, RMSD 1.34 Å over 90 aligned Ca atoms) with the notable exception that Npu and Ssp have an additional helix, which is absent in Cat.

In the Cat active site, a serine residue (Ser₇₅) replaces the threonine located in the canonical TXXH B-block motif (FIG. 9C). The carbonyl oxygen of C1A is proximal to the amide proton (2.4 Å) and the hydroxyl proton (3.7 Å) of Ser75 (FIG. 8C). The threonine residue in DnaE inteins adopts a similar conformation, suggesting that Ser75 supplants the role of threonine in assisting the cleavage of the N-terminal scissile peptide bond. Another notable feature in the structure is the lack of an F-block histidine (FIG. 9C), and therefore resolution of the branched intermediate is likely mediated by the penultimate G-block histidine (His133).

TABLE 7
Statistics from NMR structure determination
calculations of Cat complex in solution.

| Parameter | Value |
|---|---|
| Restraints | |
| Distance restraints | 3489 |
| Unambiguous restraints | 3283 |
| Infra-residue | 1667 |
| Sequential | 642 |
| Short range | 266 |
| Long range | 708 |
| Ambiguous restraints | 206 |
| Dihedral angle restraints | 180 |
| Structure statistics | |
| NOE Violations > 0.5 Å | 12 (+/− 4) |
| Dihedral violations > 5 | 0 |
| Total Energy (kcal/mol) | −5074 (+/− 163) |
| RMSD from mean structure | |
| Backbone (all residues) | 1.99 Å(+/− 0.4) |
| Heavy atoms (all residues) | 2.52 Å(+/− 0.4) |
| Backbone (structured*) | 1.19 Å(+/− 0.3) |
| Heavy atoms (structured*) | 2.04 Å(+/− 0.3) |
| Ramachandran plot analysis | |
| Most favoured regions | 85.7% |
| Additional allowed regions 13.5% | |
| Generously allowed | 0.8% |
| regions | |
| Disallowed regions | 0.0% |

*excluding exteins and solubility tag

4. Mapping Disorder Localization in Cat

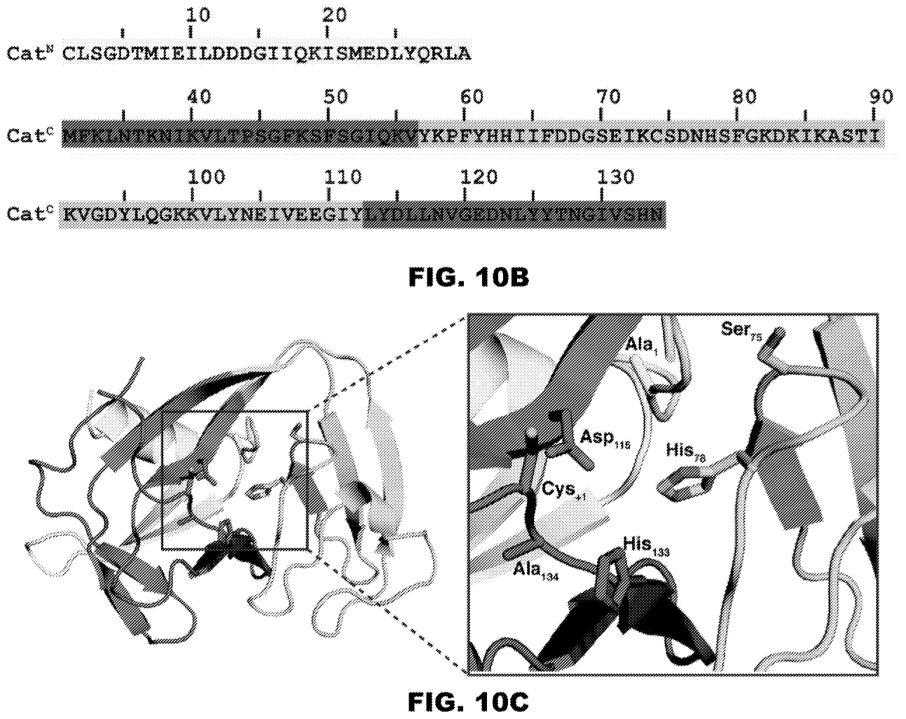

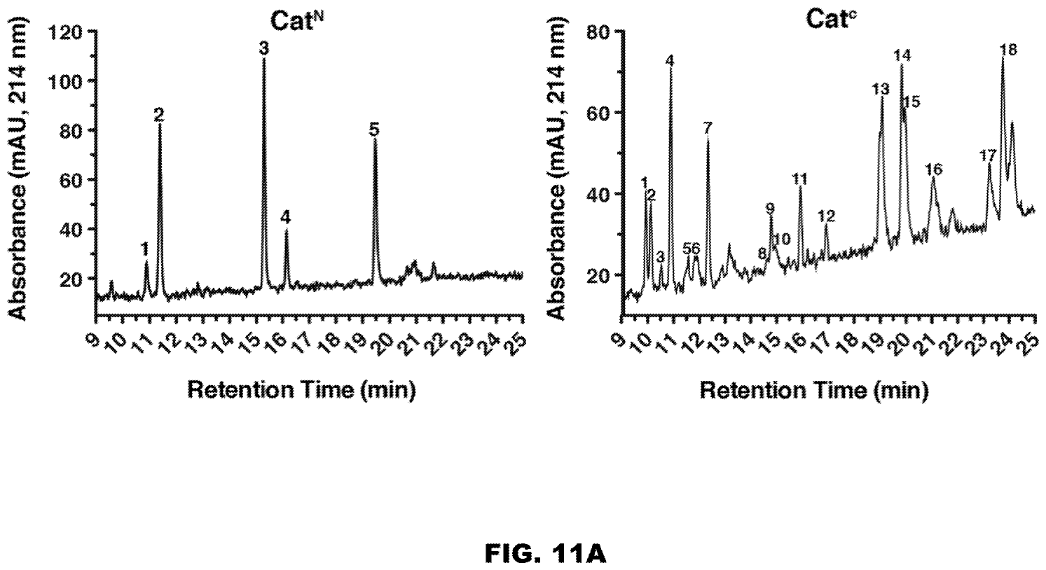

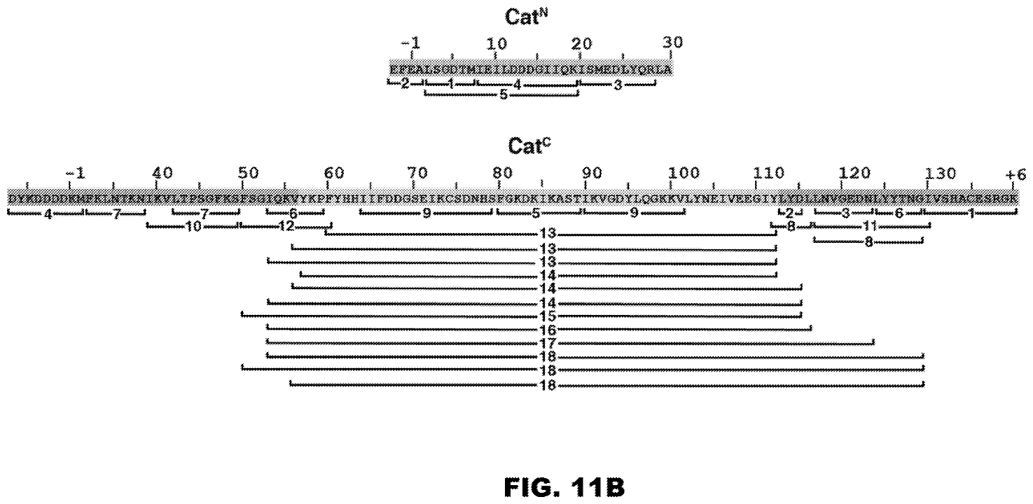

Limited proteolysis by thermolysin digestion was applied to investigate the distribution of local structure in Cat (FIG. 10A). In isolation, Cat^{N} undergoes rapid degradation, while Cat^{C} displays slightly greater resistance to proteolysis. The intein complex, however, remains intact after 30 minutes. The variation in protease susceptibility observed is consistent with a largely disordered Cat^{N}, partially disordered Cat^{C}, and formation of a globular fold upon binding. We next examined cleavage products (t=30 min) using electrospray ionization mass spectrometry (ESI-MS) to determine the regions protected from proteolysis, which should correspond to localized structural elements (FIG. 11, Table 8). For Cat^{N}, cut sites appeared to be evenly spread throughout the primary sequence. Conversely, a large portion of Cat^{C} is resistant to proteolysis. Numerous peaks corresponding to intact fragments centered on residues 57 through 112 were observed, which points to this area as a structured region flanked by disordered N- and C-terminal peptides (FIG. 10B). Mapping this model onto the structure of Cat indicates that the disordered N- and C-terminal ends of Cat^{C} directly interact with Cat^{N} (FIG. 10C). Moreover, key catalytic residues for succinimide formation (Asp115, His133, and Asn₁₃₄) are present within the disordered region of Cat^{C}.

TABLE 8
Masses from limited proteolysis.

| | | | |
|---|---|---|---|
| Peak^{a} | Mass_{obs} (Da) | Massₑₓₚ (Da) | Position |

Cat^{N}

| | | | |
|---|---|---|---|
| 1 | 623.27 | 623.27 | 2 to 7 |
| 2 | 495.21 | 495.21 | −3 to 1 |
| 3 | 1154.56 | 1154.55 | 20 to 28 |
| 4 | 1371.75 | 1371.74 | 8 to 19 |
| 5 | 1976.00 | 1975.99 | 2 to 19 |

Cat^{C}

| | | | |
|---|---|---|---|
| 1 | 1186.62 | 1186.6 | 130 to +6 |
| 2 | 410.2 | 410.19 | 113 to 115 |
| 3 | 760.35 | 760.35 | 117 to 123 |
| 4 | 1144.47 | 1144.45 | −8 to 31 |
| 5 | 1094.65 | 1094.62 | 80 to 89 |
| 6 | 875.56 | 875.53 | 53 to 59 |
| 6 | 730.34 | 730.4 | 124 to 129 |
| 7 | 864.515 | 864.49 | 32 to 38 |
| 7 | 836.45 | 836.45 | 42 to 49 |
| 8 | 686.35 | 686.34 | 112 to 116 |
| 8 | 1471.67 | 1471.68 | 117 to 129 |
| 9 | 1779.8 | 1779.79 | 64 to 79 |
| 9 | 1347.8 | 1347.82 | 90 to 101 |
| 10 | 1176.71 | 1176.7 | 39 to 49 |
| 11 | 1584.76 | 1584.75 | 117 to 130 |
| 12 | 1313.66 | 1313.76 | 50 to 60 |
| 13 | 6093.1 | 6093.8 | 60 to 112 |
| 13 | 6580.3 | 6581.4 | 56 to 112 |
| 13 | 6950.6 | 6950.9 | 53 to 112 |
| 14 | 6483.2 | 6482.3 | 57 to 112 |
| 14 | 6972.5 | 6972.9 | 56 to 115 |
| 14 | 7341.7 | 7342.3 | 53 to 115 |
| 15 | 7632.9 | 7633.6 | 50 to 115 |
| 16 | 7455.8 | 7455.5 | 53 to 116 |
| 17 | 8197.1 | 8197.2 | 53 to 123 |
| 18 | 8908.4 | 8909 | 53 to 129 |
| 18 | 9199.6 | 9200.3 | 50 to 129 |
| 18 | 8538.2 | 8539.6 | 56 to 129 |

^{a}The indicated peak number corresponds to the RP-HPLC traces in FIG. 11

5. Assembly is Largely Driven by Hydrophobic Interactions

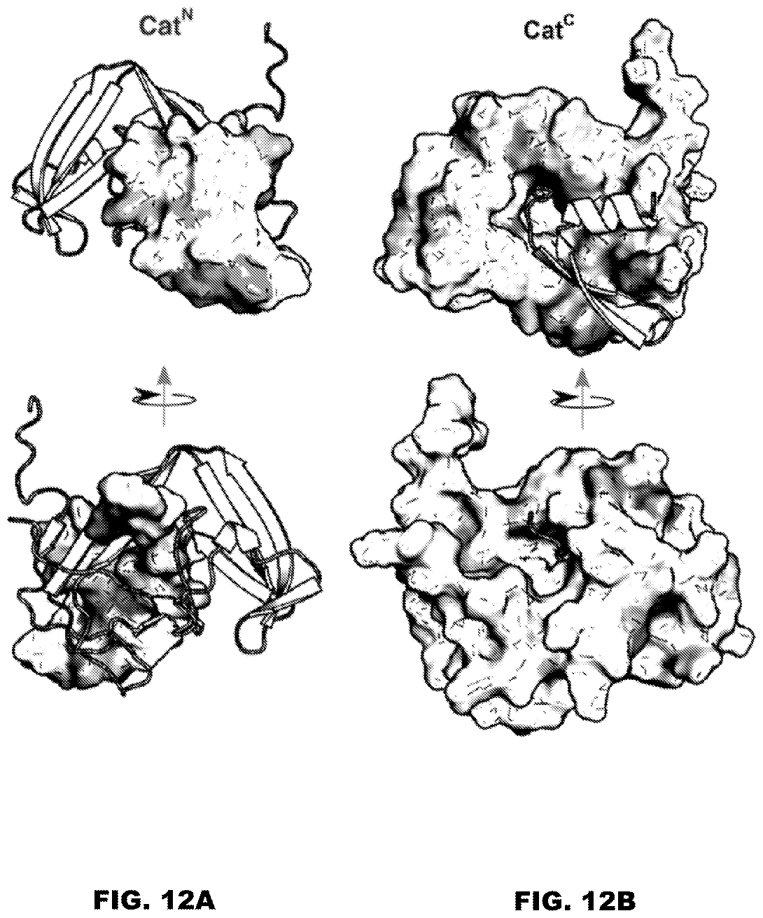

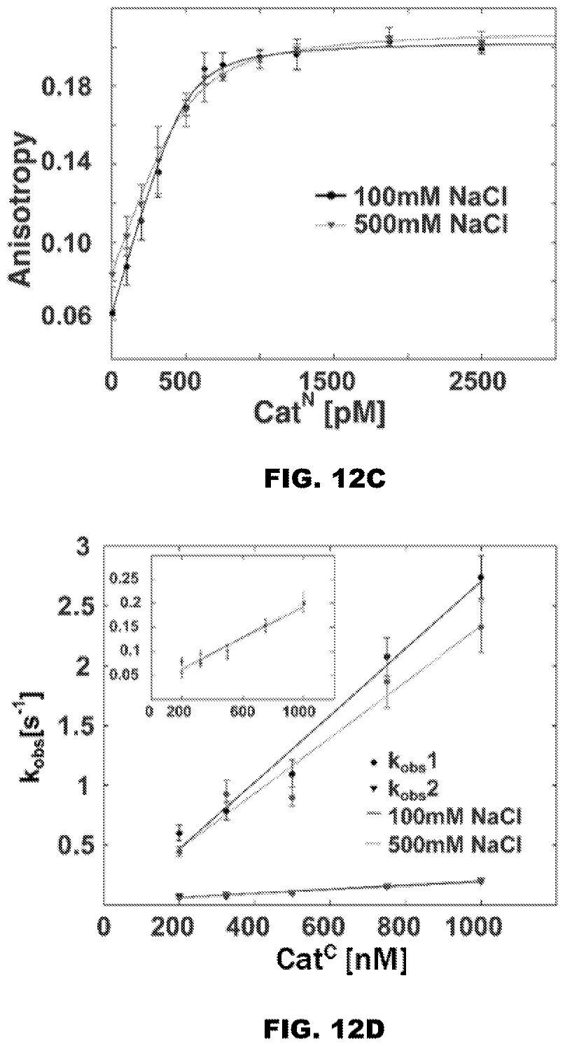

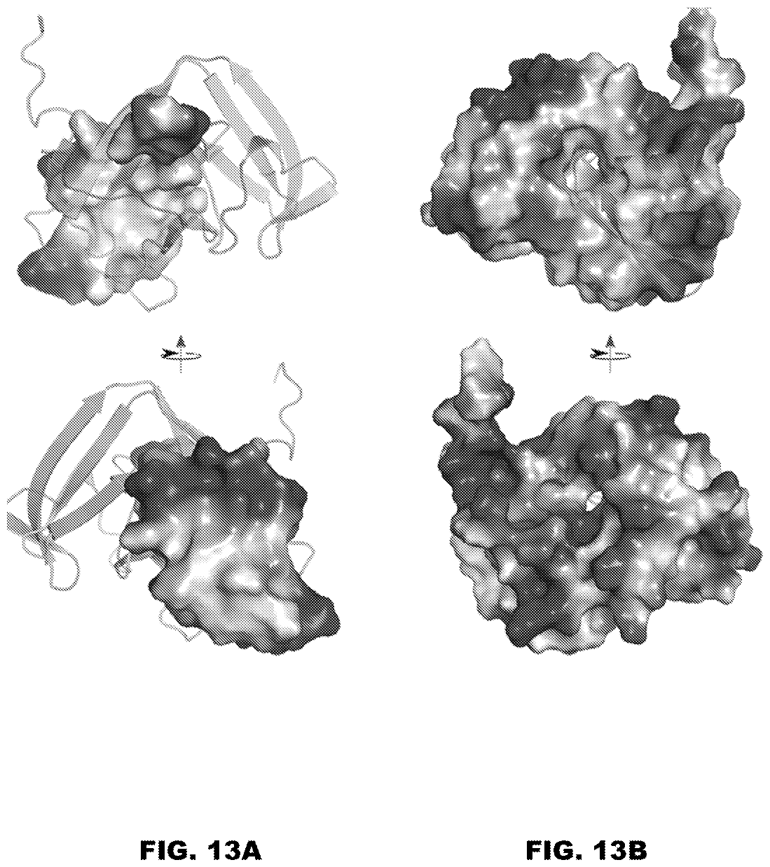

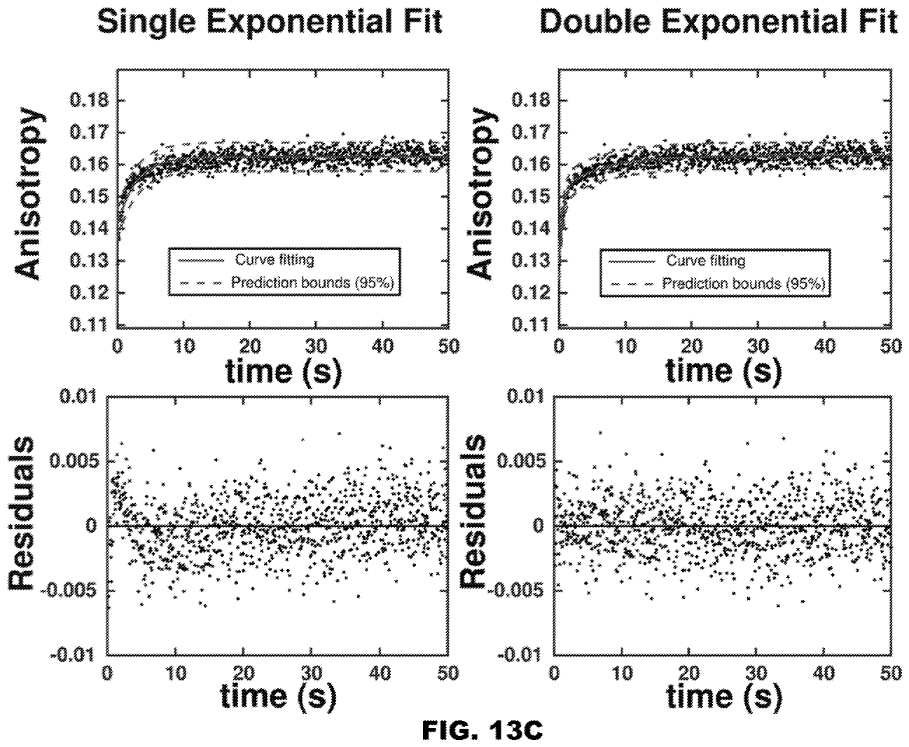

After examining the structural properties of the Cat fragments in split form, identification the molecular components that drive association were sought. Although the primary sequences of Cat^{N} and Cat^{C} exhibit separation of charge, the binding surface of Cat^{N}-Cat^{C} is rich in hydrophobic residues (FIGS. 12A and B). In the complex, the charged residues of both Cat^{N} and Cat^{C} are excluded towards the exterior of the protein while hydrophobic residues are clustered within the binding interface (FIGS. 13A and B). To validate that these hydrophobic interactions drive complex formation, the effect of buffer ionic strength on fragment association was evaluated using a fluorescence anisotropy-based binding assay. Cat^{N} containing an N-terminal fluorescein (FI-Cat^{N}) was synthesized by solid phase peptide synthesis, and an increase in fluorescence anisotropy was observed upon association with a SUMO-Cat^{C} fusion protein (FIG. 12C. This increased anisotropy is consistent with an expected increase in rotational correlation time for the Cat complex compared to unbound Cat^{N}, and was used as a measure of Cat complex formation. Like other split inteins, Cat^{N} and Cat^{C} exhibit high binding affinity in vitro, with Kd values below 500 pM, which was the limit of detection of the assay (Table 9). Importantly, the binding isotherm for Cat complex formation is minimally perturbed by a change in ionic strength of the buffer, consistent with an association process driven by hydrophobic interactions.

Kinetics of binding between FI-Cat^{N} and SUMO-Cat^{C} were next monitored by stopped-flow fluorescence, and the data was found to be best fit to a double exponential model (FIG. 13C). Both determined rate constants (kobs1 and kobs2) exhibit concentration dependence leading to a calculated kon1 of (2.80±0.28)×106 M-1 s-1 and kon2 of (0.16±0.019)×106 M-1 s-1 under low salt conditions and kon1 of (2.34±0.30)×106 M-1 s-1 and kon2 of (0.18±0.016)×106 M-1 s-1 under high salt conditions (FIG. 12D, Table 4). This model suggests that parallel association events may proceed from distinct conformers of the intein, with subsets of conformers being kinetically distinguishable. Moreover, the observation that both kobs1 and kobs2 are unperturbed by buffer ionic strength across all measured Cat^{C} concentrations further suggests that association is largely driven by hydrophobic interactions.

TABLE 9
Steady state Binding Constants.

| | 100 mM NaCl | 500 mM NaCl |
|---|---|---|
| [Cat^{C}] (pM) | Anisotropy | Anisotropy |
| 0 | 0.064 ± 0.001 | 0.084 ± 0.006 |
| 100 | 0.087 ± 0.009 | 0.103 ± 0.010 |
| 200 | 0.011 ± 0.010 | 0.120 ± 0.009 |
| 312.5 | 0.136 ± 0.013 | 0.142 ± 0.017 |
| 500 | 0.169 ± 0.004 | 0.168 ± 0.009 |
| 625 | 0.189 ± 0.008 | 0.185 ± 0.013 |
| 750 | 0.191 ± 0.006 | 0.185 ± 0.002 |
| 1000 | 0.195 ± 0.003 | 0.194 ± 0.005 |
| 1250 | 0.196 ± 0.008 | 0.199 ± 0.002 |
| 1875 | 0.203 ± 0.002 | 0.205 ± 0.005 |
| 2500 | 0.199 ± 0.003 | 0.203 ± 0.005 |
| Fit | K_{d} (pM) | K_{d} (pM) |
| | 33.87 ± 8.69 | 80.41 ± 15.41 |

6. The Extein Dependence of Cat

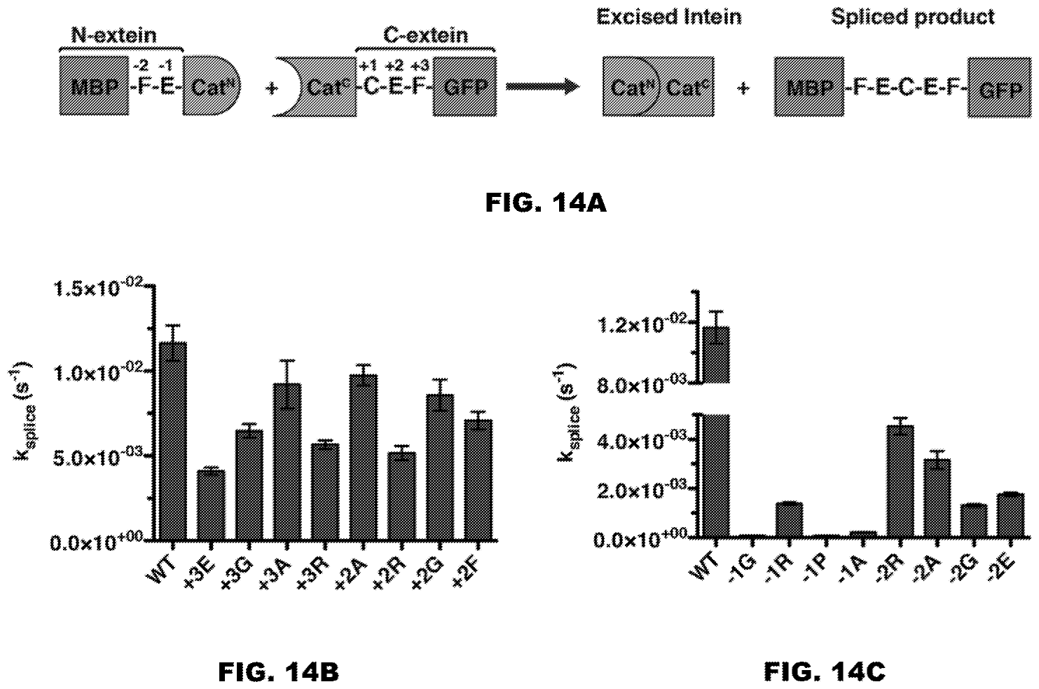

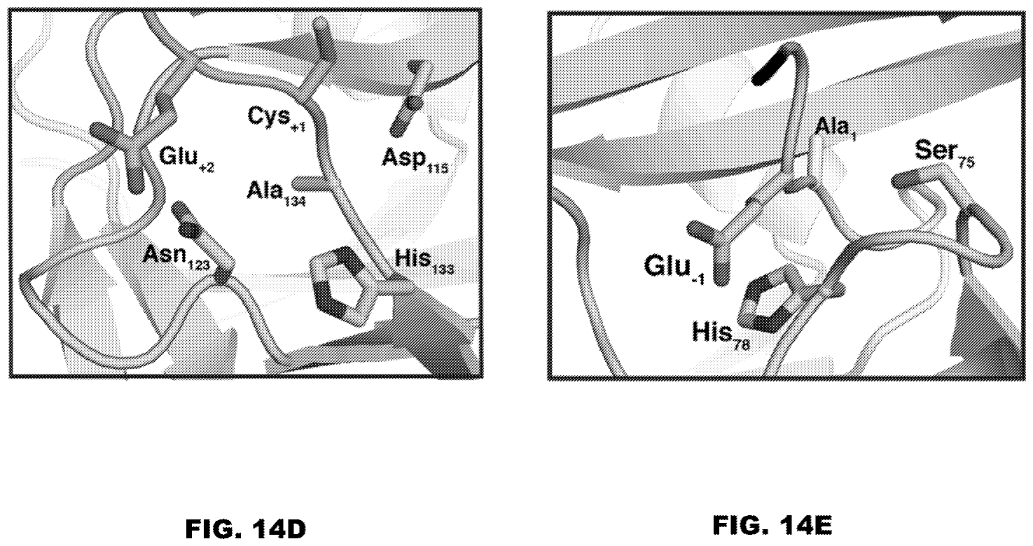

To date, all characterized inteins exhibit splicing rates dependent on their flanking extein residues. Deviation from the native extein sequence often decelerates splicing and consequently may limit applications of PTS. The extein dependence of TerL inteins has yet to be thoroughly characterized, and we therefore sought to identify the sequence preferences of Cat by introducing substitutions that vary charge and steric bulk from the native residues (FIG. 14A). Substitutions from the native C-extein, which is Cys+1, Glu+2, Phe+3, were introduced at the +2 and +3 positions and assayed in vitro (FIG. 14B, Table 10). Cat demonstrates remarkable C-extein promiscuity, splicing with half-lives ranging from 1 to 3 minutes. This broad tolerance to C-extein substitutions is superior even to an engineered version of Npu previously designed to possess promiscuous activity. Unlike the tolerance to C-extein substitution, Cat exhibits a stark dependence on the identity of the −1 residue: decreased activity results from inserting alanine (t1/2=54 min), glycine (t1/2=146 min), or proline (t1/2=158 min) at this position (FIG. 14C, Table 10). The measured in vitro extein dependence is likely explained by interactions observed in the solution structure of the Cat complex. Both Glu+2 and Phe+3 appear to have minimal contact with active site-catalytic residues, agreeing with the experimentally observed C-extein promiscuity (FIG. 14D). Interestingly, Glu+2 does contact Asn123, which is present in place of an F-block histidine. Conversely, Glu-1 directly interacts with Ser75 and His78, two conserved residues with implications in thioester formation (FIG. 14E). N-extein substitutions may therefore directly interfere with the capability of Ser75 and His78 to catalyze protein splicing.

TABLE 10
Protein splicing of Cat in varying Extein Contexts.

| ^{a}N- | ^{a}C- | | |
|---|---|---|---|
| extein₋₂, ₋₁ | Extein_{+1, +2, +3} | k_{splice} (s⁻¹) | t_{1/2} (s) |
| F [symbol] | CEF | (2.14 ± .08) × 10⁻⁴ | 3244 ± 116 |
| F [symbol] | CEF | (7.33 ± .44) × 10⁻⁵ | 9451 ± 575 |
| F [symbol] | CEF | (7.92 ± .33) × 10⁻⁵ | 8749 ± 364 |
| F [symbol] | CEF | (1.38 ± .06) × 10⁻³ | 504 ± 22 |
| [symbol] E | CEF | (4.53 ± .33) × 10⁻³ | 153 ± 11 |
| [symbol] E | CEF | (3.16 ± .36) × 10⁻³ | 220 ± 25 |
| [symbol] E | CEF | (1.30 ± .06) × 10⁻³ | 532 ± 23 |
| [symbol] E | CEF | (1.76 ± .06) × 10⁻³ | 394 ± 14 |
| FE | C [symbol] F | (9.75 ± .60) × 10⁻³ | 71 ± 4 |
| FE | C [symbol] F | (8.57 ± .91) × 10⁻³ | 80 ± 9 |
| FE | C [symbol] F | (5.16 ± .42) × 10⁻³ | 134 ± 11 |
| FE | C [symbol] F | (7.08 ± .51) × 10⁻³ | 98 ± 7 |
| FE | CE [symbol] | (6.47 ± .40) × 10⁻³ | 107 ± 7 |
| FE | CE [symbol] | (4.23 ± .23) × 10⁻³ | 164 ± 9 |
| FE | CE [symbol] | (9.20 ± 1.42) × 10⁻³ | 75 ± 12 |
| FE | CE [symbol] | (5.65 ± .25) × 10⁻³ | 123 ± 5 |

^{a}The position of mutation from the wild type extein sequence is underlined.

What is claimed is:

1. A split intein N-fragment comprising the amino acid sequence of SEQ ID NO: 1 or a variant thereof having at least 90% sequence identity with SEQ ID NO: 1.

2. The split intein N-fragment of claim 1, wherein the variant comprises an amino acid sequence selected from the group consisting of SEQ ID NO: 2-6, 125-127 and 168-170.

3. The split intein N-fragment of claim 2, wherein the variant is a functionally equivalent variant of SEQ ID NO: 1.

4. The split intein N-fragment of claim 3, wherein the functionally equivalent variant comprises the amino acid sequence of SEQ ID NO: 4 or SEQ ID NO: 125.

5. A complex comprising:
(i) a compound of interest,
(ii) the split intein N-fragment of any one of claims 1 to 4, or a split intein N-fragment comprising the amino acid sequence selected from the group consisting of SEQ ID NO: 103-110,
wherein the complex optionally comprises a linker between (i) and (ii) and
wherein
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.

6. The complex of claim 5, wherein the split intein N-fragment comprises an amino acid sequence selected from the group consisting of SEQ ID NO: 49-68 or a variant thereof.

7. The complex of any one of claim 5 or 6, wherein the compound of interest is a polypeptide or protein, and wherein if the complex comprises a linker, the linker is a peptide linker.

8. The complex of claim 7, wherein the polypeptide of interest is an antibody or a fragment of a protein.

9. The complex of claim 8, wherein the compound of interest is an N-terminal fragment of a protein.

10. A polynucleotide encoding the split intein N-fragment of any one of claims 1 to 5 or the complex of claim 7.

11. A vector comprising the polynucleotide of claim 10.

12. A host cell comprising the polynucleotide of claim 10 or the vector of claim 11.

13. A split intein C-fragment comprising the amino acid sequence of SEQ ID NO: 7 or a variant thereof having at least 88% sequence identity with SEQ ID NO: 7.

14. The split intein C-fragment of claim 13, wherein the variant comprises an amino acid sequence selected from the group consisting of SEQ ID NO: 8-48 and 128-166.

15. The split intein C-fragment of claim 14, wherein the variant is a functionally equivalent variant.

16. The split intein C-fragment of claim 15, wherein the functionally equivalent variant comprises an amino acid sequence selected from the group consisting of SEQ ID NO: 10-22 and 128-140.

17. A complex comprising:
(i) the split intein C-fragment of any one of claims 13 to 16 or a split intein C-fragment comprising a sequence selected from the group consisting of SEQ ID NO: 114-120 and
(ii) a compound of interest
wherein the complex optionally comprises a linker between (i) and (ii) and
wherein
the compound of interest is bound to the C-terminus of the split intein C-fragment by an amide linkage or
if the complex comprises a linker, the compound of interest is bound to the linker by an amide linkage and/or the linker is bound to the C-terminus of the split intein C-fragment by an amide linkage.

18. The complex of claim 17, wherein the split intein C-fragment comprises a sequence selected from SEQ ID NO: 69-87 or a variant thereof.

19. The complex of any one of claim 17 or 18, wherein the compound of interest is a polypeptide or protein, and wherein if the complex comprises a linker, the linker is a peptide linker.

20. The complex of claim 19, wherein the compound of interest is an antibody or a fragment of a protein.

21. The complex of claim 20, wherein the compound of interest is the C-terminal fragment of a protein.

22. A complex comprising:
(i) the split intein C-fragment of any one of claims 13 to 16 or a split intein C-fragment comprising a sequence selected from the group consisting of SEQ ID NO: 114-120
(ii) a compound of interest and
(iii) the split intein N-fragment of any one of claims 1 to 4, or a split intein N-fragment comprising the amino acid sequence selected from the group consisting of SEQ ID NO: 103-110
wherein the complex optionally comprises a linker between (i) and (ii) and/or between (ii) and (iii),
wherein
the compound of interest is linked to the C-terminus of the split intein C-fragment by an amide linkage or
if the complex comprises a linker, the compound of interest is bound to the linker by an amide linkage and/or the linker is bound to the C-terminus of the split intein C-fragment by an amide linkage
and
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.

23. A polynucleotide encoding the split intein C-fragment of any one of claims 13 to 16 or the complex of claim 19 or the complex of claim 22 wherein the conjugate of interest is a protein, and wherein if the complex comprises a linker, the linker is a peptide linker.

24. A vector comprising the polynucleotide of claim 23.

25. A host cell comprising the polynucleotide of claim 23 or the vector of claim 24.

26. A composition comprising the complex of any one of claims 5 to 9 and the complex of any one of claims 17 to 21.

27. A conjugate comprising the complex of any one of claims 5 to 9 and the complex of any one of claims 17 to 21, wherein the C-terminus of the split intein N-fragment is linked to the N-terminus of the split intein C-fragment by a peptide bond.

28. A conjugate comprising (a) the complex of claim 7 and (b) a split intein C-fragment comprising the amino acid sequence of SEQ ID NO: 7 or a variant thereof having at least 88% sequence identity with SEQ ID NO: 7 or an amino acid sequence selected from the group consisting of SEQ ID NO: 114-120, wherein the C-terminus of the split intein N-fragment is linked to the N-terminus of the split intein C-fragment by a peptide bond.

29. A polynucleotide encoding the conjugate of claim 28 or a vector comprising said polynucleotide.

30. A host cell comprising the polynucleotide or the vector of claim 29.

31. A method to obtain a conjugate between a first compound of interest and a second compound of interest comprising
(i) contacting
(a) the complex of any one of claims 5 to 9, wherein the complex comprises the first compound of interest and a split intein N-fragment comprising the amino sequence of SEQ ID NO: 1 or a functionally equivalent variant thereof having at least 90% sequence identity with SEQ ID NO: 1, or an amino acid sequence selected from the group consisting of SEQ ID NO: 103-110 with
(b) the complex of any one of claims 17 to 21, wherein the complex comprises the second compound of interest and a split intein C-fragment comprising the amino acid sequence of SEQ ID NO: 7 or a functionally equivalent variant thereof having at least 88% sequence identity with SEQ ID NO: 7, or an amino acid sequence selected from the group consisting of SEQ ID NO: 114-120
or
a complex comprising an AceL-TerL split intein C-fragment or a functionally equivalent variant thereof and the second compound of interest, wherein the complex optionally comprises a linker between the split intein C-fragment and the second compound of interest and wherein
the second compound of interest is bound to the C-terminus of the split intein C-fragment by an amide linkage or
if the complex comprises a linker, the second compound of interest is bound to the linker by an amide linkage and/or the linker is bound to the C-terminus of the split intein C-fragment by an amide linkage under appropriate conditions for binding the split intein N-fragment to the split intein C-fragment to form an intein intermediate and
(ii) allowing the intein intermediate to react to form a conjugate between the first and the second compound of interest.

32. A method to obtain a conjugate between a first compound of interest and a second compound of interest comprising
(i) contacting
(a) the complex of any one of claims 5 to 9, wherein the complex comprises the first compound of interest and a split intein N-fragment comprising the amino sequence of SEQ ID NO: 1 or a functionally equivalent variant thereof having at least 90% sequence identity with SEQ ID NO: 1, or an amino acid sequence selected from the group consisting of SEQ ID NO: 103-110
or
a complex comprising the second compound of interest and an AceL-TerL split intein N-fragment or a functionally equivalent variant thereof, wherein the complex optionally comprises a linker between the compound of interest and the split intein N-fragment, and wherein
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.
with
(b) the complex of any one of claims 17 to 21, wherein the complex comprises the second compound of interest and a split intein C-fragment comprising the amino acid sequence of SEQ ID NO: 7 or a functionally equivalent variant thereof having at least 88% sequence identity with SEQ ID NO: 7, or an amino acid sequence selected from the group consisting of SEQ ID NO: 114-120
under appropriate conditions for binding the split intein N-fragment to the split intein C-fragment to form an intein intermediate and
(ii) allowing the intein intermediate to react to form a conjugate between the first and the second compound of interest.

33. A method to obtain a conjugate of a compound of interest with a nucleophile comprising
(i) contacting
(a) the complex of any one of claims 5 to 9, wherein the split intein N-fragment comprises the amino acid sequence of SEQ ID NO: 1 or a functionally equivalent variant thereof having at least 90% sequence identity with SEQ ID NO: 1 or an amino acid sequence selected from the group consisting of SEQ ID NO: 103-110
or
a complex comprising a compound of interest and an AceL-TerL split intein N-fragment or a functionally equivalent variant thereof, wherein the complex optionally comprises a linker between the compound of interest and the split intein N-fragment, and wherein
the compound of interest is linked to the N-terminus of the split intein N-fragment by an amide linkage or
if the complex comprises a linker, the compound of interest is bound to the linker by an amide linkage and/or the linker is bound to the N-terminus of the split intein N-fragment by an amide linkage.
with
(b) a split intein C-fragment comprising an amino acid sequence selected from the group consisting of SEQ ID NO: 8, 9, 23-48 and 141-166, under appropriate conditions for binding between the split intein N-fragment and the split intein C-fragment to form an intein intermediate and
(ii) contacting the intein intermediate with an exogenous nucleophile.

34. The method of claim 33, further comprising contacting the conjugate of the compound of interest and the nucleophile with a second exogenous nucleophile.

35. The method of claim 34, wherein the nucleophile is a thiol.

36. The method of any one of claims 31 to 35, wherein the split intein N-fragment comprises a sequence selected from the group consisting of SEQ ID NO: 49-68 or a functionally equivalent variant thereof.

37. The method of any one of claims 31 to 35, wherein the split intein C-fragment comprises a sequence selected from the group consisting of SEQ ID NO: 69-87 or a functionally equivalent variant thereof.

38. A composition comprising:
(a) a first polynucleotide encoding a first fusion protein comprising, from the N-terminus to the C-terminus:
a first polypeptide of interest and
a split intein N-fragment comprising the sequence of SEQ ID NO: 1 or a variant thereof having at least 90% sequence identity with SEQ ID NO: 1, or an amino acid sequence selected from the group consisting of SEQ ID NO: 103-110 and
(b) a second polynucleotide encoding a second fusion protein comprising, from the N-terminus to the C-terminus:
an AceL-TerL split intein C-fragment or a variant thereof or a split intein C-fragment comprising the sequence of SEQ ID NO: 7 or a variant thereof having at least 88% sequence identity with SEQ ID NO: 7 or an amino acid sequence selected from the group consisting of SEQ ID NO: 114-120 and
a second polypeptide of interest
or
(a) a first polynucleotide encoding a first fusion protein comprising, from the N-terminus to the C-terminus:
a first polypeptide of interest and
an AceL-TerL split intein N-fragment or a variant thereof, or a split intein N-fragment comprising the sequence of SEQ ID NO: 1 or a variant thereof having at least 90% sequence identity with SEQ ID NO: 1, or an amino acid sequence selected from the group consisting of SEQ ID NO: 103-110 and
(b) a second polynucleotide encoding a second fusion protein comprising, from the N-terminus to the C-terminus:
a split intein C-fragment comprising the sequence of SEQ ID NO: 7 or a variant thereof having at least 88% sequence identity with SEQ ID NO: 7 or an amino acid sequence selected from the group consisting of SEQ ID NO: 114-120 and
a second polypeptide of interest.

39. The composition of claim 38, wherein the first polypeptide of interest is the N-terminal fragment of a protein and the second polypeptide of interest is the C-terminal fragment of said protein, and wherein upon covalently linking the C-terminus of the first polypeptide of interest to the N-terminus of the second polypeptide of interest the whole protein is obtained.

40. The composition of any one of claim 38 or 39, wherein the split intein N-fragment comprises a sequence selected from the group consisting of SEQ ID NO: 49-68 or a functionally equivalent variant thereof.

41. The composition of any one of claim 38 or 39, wherein the split intein C-fragment comprises a sequence selected from the group consisting of SEQ ID NO: 69-87 or a functionally equivalent variant thereof.

42. A method for expressing a gene of interest in a cell comprising:
(i) contacting the cell with
(a) a first polynucleotide encoding a first fusion protein comprising, from the N-terminus to the C-terminus:
a first polypeptide of interest and
a split intein N-fragment comprising the sequence of SEQ ID NO: 1 or a functionally equivalent variant thereof having at least 90% or an amino acid sequence selected from the group consisting of SEQ ID NO: 103-110, and
(b) a second polynucleotide encoding a second fusion protein comprising, from the N-terminus to the C-terminus:
an AceL-TerL split intein C-fragment or a functionally equivalent variant thereof, or a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
second polypeptide of interest,
or
(a) a first polynucleotide encoding a first fusion protein comprising, from the N-terminus to the C-terminus:
a first polypeptide of interest and
an AceL-TerL split intein N-fragment or a functionally equivalent variant thereof, or a split intein N-fragment comprising the sequence of SEQ ID NO: 1 or a functionally equivalent variant thereof having at least 90% or an amino acid sequence selected from the group consisting of SEQ ID NO: 103-110, and
(b) a second polynucleotide encoding a second fusion protein comprising, from the N-terminus to the C-terminus:
a split intein C-fragment comprising the sequence of SEQ ID NO: 7 or a variant thereof having at least 88% sequence identity with SEQ ID NO: 7, or an amino acid sequence selected from the group consisting of SEQ ID NO: 114-120, and a
second polypeptide of interest,
(ii) allowing the expression of the first and the second polynucleotides so that the first and the second fusion proteins are produced and
(iii) allowing the contact between the first and second fusion proteins so that the split intein N-fragment binds to the split intein C-fragment to form a intein intermediate and the intein intermediate reacts to covalently link the C-terminus of the first polypeptide of interest to the N-terminus of the second polypeptide of interest.

43. A method for expressing a gene of interest comprising:
(i) contacting a first cell with a first polynucleotide encoding a first fusion protein comprising, from the N-terminus to the C-terminus:
a first polypeptide of interest and
a split intein N-fragment comprising the sequence of SEQ ID NO: 1 or a functionally equivalent variant thereof having at least 90% or an amino acid sequence selected from the group consisting of SEQ ID NO: 103-110,
wherein the first fusion protein comprises a signal peptide, and
(ii) contacting a second cell with a second polynucleotide encoding a second fusion protein comprising, from the N-terminus to the C-terminus:
an AceL-TerL split intein C-fragment or a functionally equivalent variant thereof, or a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
second polypeptide of interest
wherein the second fusion protein comprises a signal peptide,
or
(i) contacting a first cell with a first polynucleotide encoding a first fusion protein comprising, from the N-terminus to the C-terminus:
a first polypeptide of interest and
an AceL-TerL split intein N-fragment or a functionally equivalent variant thereof, or a split intein N-fragment comprising the sequence of SEQ ID NO: 1 or a functionally equivalent variant thereof having at least 90% or an amino acid sequence selected from the group consisting of SEQ ID NO: 103-110,
wherein the first fusion protein comprises a signal peptide, and
(ii) contacting a second cell with a second polynucleotide encoding a second fusion protein comprising, from the N-terminus to the C-terminus:
a split intein C-fragment comprising the sequence of SEQ ID NO: 7 or a functionally equivalent variant thereof having at least 88% sequence identity with SEQ ID NO: 7, or an amino acid sequence selected from the group consisting of SEQ ID NO: 114-120, and a
second polypeptide of interest
wherein the second fusion protein comprises a signal peptide,
(iii) allowing the expression of the first and the second polynucleotides so that the first and the second fusion proteins are produced and secreted,
(iv) allowing the contact between the first and second fusion proteins so that the split intein N-fragment binds to the split intein C-fragment to form a intein intermediate and the intein intermediate reacts to covalently link the C-terminus of the first polypeptide of interest to the N-terminus of the second polypeptide of interest. The method of any one of claim 42 or 43, wherein the first polypeptide of interest is the N-terminal fragment of a protein and the second polypeptide of interest is the C-terminal fragment of said protein, and wherein upon covalently linking the C-terminus of the first polypeptide of interest to the N-terminus of the second polypeptide of interest the whole protein is obtained.

44. The method of any one of claims 42 to 44, wherein the split intein N-fragment comprises a sequence selected from the group consisting of SEQ ID NO: 49-68 or a functionally equivalent variant thereof.

45. The method of any one of claims 44 to 44, wherein the split intein C-fragment comprises a sequence selected from the group consisting of SEQ ID NO: 69-87 or a functionally equivalent variant thereof.


SEQUENCE LISTING (170 sequences)

SEQ ID NO: 1 - protein, 30 aa, Artificial Sequence
  cat N
CLSGDTMIEILDDDGIIQKISMEDLYQRLA

SEQ ID NO: 2 - protein, 30 aa, Artificial Sequence
  cat N variant
  Xaa (1)..(1): Xaa any amino acid other than Cys, Ser or Thr
XLSGDTMIEILDDDGIIQKISMEDLYQRLA

SEQ ID NO: 3 - protein, 30 aa, Artificial Sequence
  cat N variant
ALSGDTMIEILDDDGIIQKISMEDLYQRLA

SEQ ID NO: 4 - protein, 33 aa, Artificial Sequence
  cat N variant
EFECLSGDTMIEILDDDGIIQKISMEDLYQRLA

SEQ ID NO: 5 - protein, 33 aa, Artificial Sequence
  cat N variant
  MISC_FEATURE (4)..(4): Xaa any amino acid other than Cys, Ser or Thr
EFEXLSGDTMIEILDDDGIIQKISMEDLYQRLA

SEQ ID NO: 6 - protein, 33 aa, Artificial Sequence
  cat N variant
EFEALSGDTMIEILDDDGIIQKISMEDLYQRLA

SEQ ID NO: 7 - protein, 104 aa, Artificial Sequence
  cat C
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHN

SEQ ID NO: 8 - protein, 104 aa, Artificial Sequence
  cat C variant
  Xaa (104)..(104): Xaa any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHX

SEQ ID NO: 9 - protein, 104 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHA

SEQ ID NO: 10 - protein, 108 aa, Artificial Sequence
  cat C variant
  misc_feature (106)..(107): Xaa can be any naturally occurring amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCXXL

SEQ ID NO: 11 - protein, 108 aa, Artificial Sequence
  cat C variant
  Xaa (106)..(106): Xaa is Ala, Gly, Arg or Phe
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCXZL

SEQ ID NO: 12 - protein, 108 aa, Artificial Sequence; identical to SEQ ID NO: 10
  cat C variant
  misc_feature (106)..(106): Xaa can be any naturally occurring amino acid
  Xaa (107)..(107): Xaa is Gly, Glu, Ala or Arg

SEQ ID NO: 13 - protein, 108 aa, Artificial Sequence; identical to SEQ ID NO: 10
  cat C variant
  Xaa (106)..(106): Xaa is Ala, Gly, Arg or Phe
  Xaa (107)..(107): Xaa is Gly, Glu, Ala or Arg

SEQ ID NO: 14 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEFL

SEQ ID NO: 15 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCAFL

SEQ ID NO: 16 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCGFL

SEQ ID NO: 17 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCRFL

SEQ ID NO: 18 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCFFL

SEQ ID NO: 19 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEGL

SEQ ID NO: 20 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEEL

SEQ ID NO: 21 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEAL

SEQ ID NO: 22 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCERL

SEQ ID NO: 23 - protein, 108 aa, Artificial Sequence
  cat C variant
  Xaa (104)..(104): Xaa is any amino acid other than Ans or Gln
  misc_feature (106)..(107): Xaa can be any naturally occurring amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCXXL

SEQ ID NO: 24 - protein, 108 aa, Artificial Sequence; identical to SEQ ID NO: 23
  cat C variant
  Xaa (104)..(104): Xaa is any amino acid other than Ans or Gln
  Xaa (106)..(106): Xaa is Ala, Gly, Arg or Phe
  misc_feature (107)..(107): Xaa can be any naturally occurring amino acid

SEQ ID NO: 25 - protein, 108 aa, Artificial Sequence; identical to SEQ ID NO: 23
  cat C variant
  Xaa (104)..(104): Xaa is any amino acid other than Ans or Gln
  misc_feature (106)..(106): Xaa can be any naturally occurring amino acid
  Xaa (107)..(107): Xaa is Gly, Glu, Ala or Arg

SEQ ID NO: 26 - protein, 108 aa, Artificial Sequence; identical to SEQ ID NO: 23
  cat C variant
  Xaa (104)..(104): Xaa is any amino acid ither than Ans or Gln
  Xaa (106)..(106): Xaa is Ala, Gly, Arg or Phe
  Xaa (107)..(107): Xaa is Gly, Glu, Ala or Arg

SEQ ID NO: 27 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEFL

SEQ ID NO: 28 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCAFL

SEQ ID NO: 29 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCGFL

SEQ ID NO: 30 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCRFL

SEQ ID NO: 31 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCFFL

SEQ ID NO: 32 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEGL

SEQ ID NO: 33 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEEL

SEQ ID NO: 34 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEAL

SEQ ID NO: 35 - protein, 108 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCERL

SEQ ID NO: 36 - protein, 108 aa, Artificial Sequence
  cat C variant
  misc_feature (106)..(107): Xaa can be any naturally occurring amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACXXL

SEQ ID NO: 37 - protein, 108 aa, Artificial Sequence; identical to SEQ ID NO: 36
  cat C variant
  Xaa (106)..(106): Xaa is Ala, Gly, Arg or Phe
  misc_feature (107)..(107): Xaa can be any naturally occurring amino acid

SEQ ID NO: 38 - protein, 108 aa, Artificial Sequence; identical to SEQ ID NO: 36
  cat C variant
  misc_feature (106)..(106): Xaa can be any naturally occurring amino acid
  Xaa (107)..(107): Xaa is Gly, Glu, Ala or Arg

SEQ ID NO: 39 - protein, 108 aa, Artificial Sequence; identical to SEQ ID NO: 36
  cat C variant
  Xaa (106)..(106): Xaa is Ala, Gly, Arg or Phe
  Xaa (107)..(107): Xaa is Gly, Glu, Ala or Arg

SEQ ID NO: 40 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEFL

SEQ ID NO: 41 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACAFL

SEQ ID NO: 42 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACGFL

SEQ ID NO: 43 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACRFL

SEQ ID NO: 44 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACFFL

SEQ ID NO: 45 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEGL

SEQ ID NO: 46 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEEL

SEQ ID NO: 47 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEAL

SEQ ID NO: 48 - protein, 108 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACERL

SEQ ID NO: 49 - protein, 29 aa, Artificial Sequence
  AAC N intein
CLGGDTIIEIQDDDGITQKISMEDLYERL

SEQ ID NO: 50 - protein, 29 aa, Artificial Sequence
  AAC2 N intein
CLGGDTEIEILDDNGIVQKTSMENLYERL

SEQ ID NO: 51 - protein, 29 aa, Artificial Sequence
  FUW N intein
CLGGETLIEIQDDNENISKVSMEDLYDRM

SEQ ID NO: 52 - protein, 30 aa, Artificial Sequence
  FUW2 N intein
CVDGDTIVEIYDKKTKEEYCVKIKDLYDLI

SEQ ID NO: 53 - protein, 29 aa, Artificial Sequence
  FUW3 N intein
CLSGDTQIEIKNVNDKIESVSMEELYERM

SEQ ID NO: 54 - protein, 28 aa, Artificial Sequence
  AAC3 N intein
CLSGDTMIEILDENGIPQKISMEDLYQR

SEQ ID NO: 55 - protein, 28 aa, Artificial Sequence
  MDT N intein
CVSGDTNIEIECEDGVETTTIKDLYDRM

SEQ ID NO: 56 - protein, 24 aa, Artificial Sequence
  CEP N intein
CVDGDTMVETEDGKIKIEDLYKKL

SEQ ID NO: 57 - protein, 24 aa, Artificial Sequence
  MEH N intein
CVYGDTMVETEDGKIKIEDLYKKL

SEQ ID NO: 58 - protein, 28 aa, Artificial Sequence
  MDT2 N intein
CVRGDTLVEVEKDDVISEMRIEDLYNRM

SEQ ID NO: 59 - protein, 28 aa, Artificial Sequence
  ABL N intein
CVGGNTLVEVEKDDIISEMRIEDLYNTM

SEQ ID NO: 60 - protein, 29 aa, Artificial Sequence
  SSF N intein
CLSGDTTIEILDVDGIPQKISMEDLYQRL

SEQ ID NO: 61 - protein, 29 aa, Artificial Sequence
  Del N intein
CLSGDTMIEILDESGIPQKISMKELYQRM

SEQ ID NO: 62 - protein, 29 aa, Artificial Sequence
  Del2 N intein
CLDGNTSIEILDENNTIQKISMENLYKRL

SEQ ID NO: 63 - protein, 29 aa, Artificial Sequence
  Del3 N intein
CLGGDTIIEIQDDDGITQKISMEDLYQRL

SEQ ID NO: 64 - protein, 29 aa, Artificial Sequence
  Del4 N intein
CLDGGTSIEILDTNNITQKISLENLYERL

SEQ ID NO: 65 - protein, 29 aa, Artificial Sequence
  Del5 N intein
CLSGDTSIEILDENNTIQKISMEDLYERL

SEQ ID NO: 66 - protein, 26 aa, Artificial Sequence
  Del6 N intein
CLSGDTLIEIIDDDGNTQKISMEDLY

SEQ ID NO: 67 - protein, 27 aa, Artificial Sequence
  Del7 N intein
CLSGDTLIEIIDDDGNTQKISMEDLYQ

SEQ ID NO: 68 - protein, 29 aa, Artificial Sequence
  Kab N intein
CLGGDTIIEIKDDDGITQKISMEDLYQRL

SEQ ID NO: 69 - protein, 104 aa, Artificial Sequence
  Cep C intein
MFKTNTNNIKILSPDGFSNFNGIQKVKRKLYQHIIFEGGTEIKTSINHSFGKDKILARDIKVGDYLNNKKVLYNELVNEKIFLYDPINVEKENLYITNDVVSHN

SEQ ID NO: 70 - protein, 103 aa, Artificial Sequence
  Mdt C intein
MYKVNNNIKVKTPTGFQSFSGIQKVFKPFYHWIIFDDGSEIKCSDNHSFGSEKIKASSLKLDDIIQGKKVLYNEIVEEGIYLYDLLDVGEENLYITNKIISHN

SEQ ID NO: 71 - protein, 100 aa, Artificial Sequence
  Meh C intein
MSKTYEVLSPSGFVKFSGIQKVSRSKYRHFIFDDGAELKCSLNHRFGKDEILASSLWPSSDLQGKNILYAEDVEEDIDLYDLLNVGGGNLYYTNGLVSHN

SEQ ID NO: 72 - protein, 103 aa, Artificial Sequence
  Aac C intein
MFKINKNIKVKTPDGFKDFSGIQKVYKPFYHWIIFDDGSEIKCSDNHSFGKEKIKASTIKVDDILQEKKVLYNEIVEEGIYLYDLLDVGEDNLYYSNNIVSHN

SEQ ID NO: 73 - protein, 103 aa, Artificial Sequence
  Aac2 C intein
MFKLNKNIEVKTPDGFKSFSGIQKVYKPFYHWIIFDDGSEIKCSDNHSFGKEKIKASTIRVDDFLQGKKVVYNEIVEEGIYLYDLLDVGENNLYYSNNIISHN

SEQ ID NO: 74 - protein, 103 aa, Artificial Sequence
  Cen C intein
MYKLNSSIKVKTPRGFKKFAGIQKVRKPVYQWIIFGDDSEIKCSLDHSFGEEQVKAHTIKTGDLLQHKEVVYSEIVEEPIDLYDLLEVEDGNLYNTNGVVSHN

SEQ ID NO: 75 - protein, 101 aa, Artificial Sequence
  Cep2 C intein
MYEVLSPSGFVKFSGVQKVSRSKYRHFIFDDGTEIKCSLDHRFGGLWDEDEILASSLNRGEYLQGKKILYVEDVEEQIDLYDLMNVDGGNLYYTNGLVSHN

SEQ ID NO: 76 - protein, 100 aa, Artificial Sequence
  Cep3 C intein
MSKTYEVLSPSGFVKFSGIQKVSHSKYRHFIFDDGTELKCSFNHRFGKDEILASSLCRGSDLQGKKILYAEDVEEDIDLYDLLNVGGGNLYYTNGLVSHN

SEQ ID NO: 77 - protein, 108 aa, Artificial Sequence
  Cep4 C intein
MYIRYQKTTSKTYEVLSPSGFVNFSGIQTVPHSKYRHFIFDDGTELKCSLNHRFDKDEILASSLWRGAELQGKQILYAEDIEEDIDLYDLLNVGGGNLYYTNGLVSHN

SEQ ID NO: 78 - protein, 107 aa, Artificial Sequence
  Cep5 C intein
MFTKYKILTPNGYESFDGVNRIKRDMYSHLIFSSGIEIRCSLNHPLYISKGDIIKSYELKIGDKVLSKNGWEIVTYNEIIEEPIYLYDIINSGKDHNYYTNDILSHN

SEQ ID NO: 79 - protein, 111 aa, Artificial Sequence
  Cep6 C intein
MISKNFTKYKILTPNGYESFDGVNRIKRDMYSHLIFSSGIEIRCSLNHPLYISKGDIIKSYELKIGDKVLSKNGWEIVTYNEIIEEPIYLYDIINSGKDHNYYTNDILSHN

SEQ ID NO: 80 - protein, 103 aa, Artificial Sequence
  Lak C intein
MFKLNKNIEVKTPDGFKSFSGIQKVYKPFYHWIIFDDGSEIKCSDNHSFGKEKIKASTIRVDDFLQGKKVVYNEIVEEGIYLYDLLDVGENNLYYSNDIISHN

SEQ ID NO: 81 - protein, 103 aa, Artificial Sequence
  Kab C intein
MFKLNKNIRVKTPSGFKSFSGIQKVYKPFYHWIIFDDGSEIKCSDNHSFGEEQIKASSIKVDDFLQGKKVVYNEIVEEGIYLYDLLDVGEDNLYYSNDVVSHN

SEQ ID NO: 82 - protein, 103 aa, Artificial Sequence
  Chb C intein
MFKLNKNIKVKTPRGFKFFSGIQKVYKPYYHWIIFDDGSEIKCSDNHSFGKEKTKASTIKVDDFLQGKKVVYNEIVEEGIYLYDLLDVGEDNLYYSNEIISHN

SEQ ID NO: 83 - protein, 103 aa, Artificial Sequence
  Del C intein
MFKLNKNIKVKTPSGFKYFSGIQKVYKPFYHWIIFDDGTEIKCSDNHSFGKEQIKASMIKVDDFFQGKKVVYNEIVEEEIYLYDLLDVGEDNLYFSNGIISHN

SEQ ID NO: 84 - protein, 103 aa, Artificial Sequence
  Del2 C intein
MFKLNKNIEVKTPDGFKSFSGIQKVYKPFYHWIIFDDGSEIKCSDNHSFGKEKIKASTIKVDDLLQGKKVVYNEIVEEGIYLYDLLDVGEDNLYYSNNLVSHN

SEQ ID NO: 85 - protein, 103 aa, Artificial Sequence
  Del3 C intein
MFKLNKNITVKTPSGFKSFSGIQKVYKPFYHWIIFDDGSEIKCSDNHSFGEEQIKASMIKVDDFLQGKKVVYNEVVEEGVYLYDLLDVGEDNLYYSNNIISHN

SEQ ID NO: 86 - protein, 104 aa, Artificial Sequence
  AceL2 C intein
MFRTNTDNIKILSPSGFSNFNGIQKVERDLYQHIIFDDKSEIKTSINHPFGKDKILARNIKVGDYLNSKKVLYNELVAEKITLYDPINVEKENLYITNGVISHN

SEQ ID NO: 87 - protein, 104 aa, Artificial Sequence
  AceL3 C intein
MFRTNTDNIKILSPSGFSNFNGIQKVERDLYQHIIFDDKSEIKTSINHPFGKDKILARNIKVGDYLNSKKVLYNELVNEKITLYDPINVEKENLYITNGVISHN

SEQ ID NO: 88 - protein, 152 aa, Artificial Sequence
  SUMO-catN
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGEFEALSGDTMIEILDDDGIIQKISMEDLYQRLA

SEQ ID NO: 89 - protein, 147 aa, Artificial Sequence
  SUMO-AceL*N
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGEFEAVSGDTMVETEDGKIKIEDLYKRLA

SEQ ID NO: 90 - protein, 159 aa, Artificial Sequence
  SUMO-GosN
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGEFEAISQESYINIEVNGKVETIKIGDLYKKLSFNERKFNE

SEQ ID NO: 91 - protein, 227 aa, Artificial Sequence
  SUMO-CatC
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEFL

SEQ ID NO: 92 - protein, 237 aa, Artificial Sequence
  SUMO-Flag-CatC
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKMFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACESRGK

SEQ ID NO: 93 - protein, 227 aa, Artificial Sequence
  SUMO-AceL*C
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMFRTNTNNIKILGPNGFSNFIGIQKVERDQYQHIIFDDDTEIKTSINHPFGKDKILARDVKVGDYLNSKKVLYNELVNENIFLYDPINVEKESLYITNGVVSHACEFL

SEQ ID NO: 94 - protein, 238 aa, Artificial Sequence
  SUMO-GosC
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMKLPESVVKNNINLKIETPYGFENFYGVNKIKKDKYIHLEFTNGEKLKCSLDHPLSTIDGIVKAKDLDKYTEVYTKFGGCFLKKSKVINESIELYDIVNSGLKHLYYSNNIISHACEFL

SEQ ID NO: 95 - protein, 474 aa, Artificial Sequence
  AceL*C-GFP
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMFRTNTNNIKILGPNGFSNFIGIQKVERDQYQHIIFDDDTEIKTSINHPFGKDKILARDVKVGDYLNSKKVLYNELVNENIFLYDPINVEKESLYITNGVVSHNCEFLMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDYKDDDDK

SEQ ID NO: 96 - protein, 474 aa, Artificial Sequence
  CatC-GFP
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEFLMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKDYKDDDDK

SEQ ID NO: 97 - protein, 514 aa, Artificial Sequence
  MBP-AceL*N
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEAPKDAQTNEFECVSGDTMVETEDGKIKIEDLYKRLA

SEQ ID NO: 98 - protein, 519 aa, Artificial Sequence
  MBP-CatN
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEAPKDAQTNEFECLSGDTMIEILDDDGIIQKISMEDLYQRLA

SEQ ID NO: 99 - protein, 25 aa, Artificial Sequence
  AceL N
CVYGDTMVETEDGKIKIEDLYKRLA

SEQ ID NO: 100 - protein, 25 aa, Artificial Sequence
  AceL* N
CVSGDTMVETEDGKIKIEDLYKRLA

SEQ ID NO: 101 - protein, 104 aa, Artificial Sequence
  AceL C
MFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHN

SEQ ID NO: 102 - protein, 104 aa, Artificial Sequence
  AceL* C
MFRTNTNNIKILGPNGFSNFIGIQKVERDQYQHIIFDDDTEIKTSINHPFGKDKILARDVKVGDYLNSKKVLYNELVNENIFLYDPINVEKESLYITNGVVSHN

SEQ ID NO: 103 - protein, 29 aa, Artificial Sequence
  AceL-TerL N consensus sequence
  MISC_FEATURE (2)..(2): Xaa can be Ile, Val, Leu or Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu or Phe
  MISC_FEATURE (10)..(10): Xaa can be Ile, Val or Thr
  misc_feature (11)..(20): Xaa can be any naturally occurring amino acid
  MISC_FEATURE (21)..(21): Xaa can be Ser, Thr or Arg
  misc_feature (22)..(22): Xaa can be any naturally occurring amino acid
  MISC_FEATURE (23)..(23): Xaa can be Asp, Glu, Lys or Arg
  misc_feature (24)..(24): Xaa can be any naturally occurring amino acid
  misc_feature (27)..(29): Xaa can be any naturally occurring amino acid
CXXGXTXXEXXXXXXXXXXXXXXXLYXXX

SEQ ID NO: 104 - protein, 30 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(21): Xaa can be any naturally occurring amino acid
  MISC_FEATURE (22)..(22): Xaa can be Ser, Thr, Arg
  misc_feature (23)..(23): Xaa can be any naturally occurring amino acid
  MISC_FEATURE (24)..(24): Xaa can be Asp, Glu, Lys or Arg
  misc_feature (25)..(25): Xaa can be any naturally occurring amino acid
  misc_feature (28)..(30): Xaa can be any naturally occurring amino acid
CXXGXTXXEXXXXXXXXXXXXXXXXLYXXX

SEQ ID NO: 105 - protein, 28 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(20): Xaa can be any naturally occurring amino acid
  MISC_FEATURE (21)..(21): Xaa can be Ser, Thr, Arg
  misc_feature (22)..(22): Xaa can be any naturally occurring amino acid
  MISC_FEATURE (23)..(23): Xaa can be Asp, Glu, Lys or Arg
  misc_feature (24)..(24): Xaa can be any naturally occurring amino acid
  misc_feature (27)..(28): Xaa can be any naturally occurring amino acid
CXXGXTXXEXXXXXXXXXXXXXXXLYXX

SEQ ID NO: 106 - protein, 28 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(19): Xaa can be any naturally occurring amino acid
  MISC_FEATURE (20)..(20): Xaa can be Ser, Thr, Arg
  misc_feature (21)..(21): Xaa can be any naturally occurring amino acid
  MISC_FEATURE (22)..(22): Xaa can be Asp, Glu, Lys or Arg
  misc_feature (23)..(23): Xaa can be any naturally occurring amino acid
  misc_feature (26)..(28): Xaa can be any naturally occurring amino acid
CXXGXTXXEXXXXXXXXXXXXXXLYXXX

SEQ ID NO: 107 - protein, 24 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(15): Xaa can be any naturally occurring amino acid
  MISC_FEATURE (16)..(16): Xaa can be Ser, Thr, Arg
  misc_feature (17)..(17): Xaa can be any naturally occurring amino acid
  MISC_FEATURE (18)..(18): Xaa can be Asp, Glu, Lys or Arg
  misc_feature (19)..(19): Xaa can be any naturally occurring amino acid
  misc_feature (22)..(24): Xaa can be any naturally occurring amino acid
CXXGXTXXEXXXXXXXXXXLYXXX

SEQ ID NO: 108 - protein, 28 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(19): Xaa can be any naturally occurring amino acid
  MISC_FEATURE (20)..(20): Xaa can be Ser, Thr, Arg
  misc_feature (21)..(21): Xaa can be any naturally occurring amino acid
  MISC_FEATURE (22)..(22): Xaa can be Asp, Glu, Lys or Arg
  misc_feature (23)..(23): Xaa can be any naturally occurring amino acid
  misc_feature (26)..(28): Xaa can be any naturally occurring amino acid
CXXGXTXXEXXXXXXXXXXXXXXLYXXX

SEQ ID NO: 109 - protein, 26 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(20): Xaa can be any naturally occurring amino acid
  MISC_FEATURE (21)..(21): Xaa can be Ser, Thr, Arg
  misc_feature (22)..(22): Xaa can be any naturally occurring amino acid
  MISC_FEATURE (23)..(23): Xaa can be Asp, Glu, Lys or Arg
  misc_feature (24)..(24): Xaa can be any naturally occurring amino acid
CXXGXTXXEXXXXXXXXXXXXXXXLY

SEQ ID NO: 110 - protein, 27 aa, Artificial Sequence
  AceL-TerL N consensus sequence
  MISC_FEATURE (2)..(2): Xaa can be Ile, Val, Leu, Phe
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  MISC_FEATURE (8)..(8): Xaa can be Ile, Val, Leu, Phe
  MISC_FEATURE (10)..(10): Xaa can be Ile, Val, Thr
  misc_feature (11)..(20): Xaa can be any naturally occurring amino acid
  MISC_FEATURE (21)..(21): Xaa can be Ser, Thr, Arg
  misc_feature (22)..(22): Xaa can be any naturally occurring amino acid
  MISC_FEATURE (23)..(23): Xaa can be Asp, Glu, Lys or Arg
  misc_feature (24)..(24): Xaa can be any naturally occurring amino acid
  misc_feature (27)..(27): Xaa can be any naturally occurring amino acid
CXXGXTXXEXXXXXXXXXXXXXXXLYX

SEQ ID NO: 111 - protein, 30 aa, Artificial Sequence
  AceL-TerL N consensus sequence
  MISC_FEATURE (2)..(2): Xaa is Leu or Val
  MISC_FEATURE (3)..(3): Xaa is Ser, Tyr, Gly, Asp or Arg
  MISC_FEATURE (5)..(5): Xaa is Asp, Glu, Asn or Gly
  MISC_FEATURE (7)..(7): Xaa is Met, Ile, Glu, Leu, Gln, Asn, Thr or Ser
  MISC_FEATURE (8)..(8): Xaa is Ile or Val
  MISC_FEATURE (10)..(10): Xaa is Ile, Thr or Val
  MISC_FEATURE (11)..(11): Xaa is Leu, Glu, Gln, Tyr, Lys, or Ile
  MISC_FEATURE (12)..(12): Xaa is Asp, Asn, Cys or Lys
  MISC_FEATURE (13)..(13): Xaa is Asp, Gly, Glu, Val, Lys or Thr
  MISC_FEATURE (14)..(14): Xaa is Asp, Lys, Asn or Ser
  MISC_FEATURE (15)..(15): Xaa is Gly, Glu, Thr, Asp, Val, Ile or Asn
  MISC_FEATURE (16)..(16): Xaa is Ile, Asn, Lys, Val or Thr
  MISC_FEATURE (17)..(17): Xaa is Ile, Thr, Val, Glu, Pro or Ser
  MISC_FEATURE (18)..(18): Xaa is Gln, Ser, Glu or Thr
  MISC_FEATURE (19)..(19): Xaa is Lys, Cys, Ser or Glu
  MISC_FEATURE (20)..(20): Xaa is Ile, Thr, Val or Met
  MISC_FEATURE (21)..(21): Xaa is Ser, Thr or Arg
  MISC_FEATURE (22)..(22): Xaa is Met, Ile or Leu
  MISC_FEATURE (23)..(23): Xaa is Glu or Lys
  MISC_FEATURE (24)..(24): Xaa is Asp, Asn or Glu
  MISC_FEATURE (27)..(27): Xaa is Gln, Lys, Glu, Asp or Asn
  MISC_FEATURE (28)..(28): Xaa is Arg, Leu, Lys or Thr
  MISC_FEATURE (29)..(29): Xaa is Leu, Met or Ile
CXXGXTXXEXXXXXXXXXXXXXXXLYXXXA

SEQ ID NO: 112 - protein, 29 aa, Artificial Sequence
  AceL-TerL N consensus sequence with increased thermostability
  MISC_FEATURE (2)..(2): Xaa is Leu or Val
  MISC_FEATURE (4)..(4): Xaa is Asp or Asn
  MISC_FEATURE (6)..(6): Xaa is Met, Ile, Ser or Leu
  MISC_FEATURE (7)..(7): Xaa is Ile or Val
  MISC_FEATURE (9)..(9): Xaa is Ile, Thr or Val
  MISC_FEATURE (10)..(10): Xaa is Leu, Glu, Gln, Lys or Ile
  MISC_FEATURE (11)..(11): Xaa is Asp or Lys
  MISC_FEATURE (12)..(12): Xaa is Asp, Gly, Glu or Val
  MISC_FEATURE (13)..(13): Xaa is Asp, Lys or Asn
  MISC_FEATURE (14)..(14): Xaa is Gly or Asn
  MISC_FEATURE (15)..(15): Xaa is Ile, Lys, Thr, Asn or Val
  MISC_FEATURE (16)..(16): Xaa is Ile, Thr, Pro, Ser or Glu
  MISC_FEATURE (17)..(17): Xaa is Gln or Glu
  MISC_FEATURE (18)..(18): Xaa is Lys or Glu
  MISC_FEATURE (19)..(19): Xaa is Ile, Thr, Val or Met
  MISC_FEATURE (20)..(20): Xaa is Ser or Arg
  MISC_FEATURE (21)..(21): Xaa is Met, Ile or Leu
  MISC_FEATURE (22)..(22): Xaa is Glu or Lys
  MISC_FEATURE (23)..(23): Xaa is Asp, Asn or Glu
  MISC_FEATURE (26)..(26): Xaa is Gln, Lys, Glu, Asp or Asn
  MISC_FEATURE (27)..(27): Xaa is Arg or Lys
  MISC_FEATURE (28)..(28): Xaa is Leu or Met
CXGXTXXEXXXXXXXXXXXXXXXLYXXXA

SEQ ID NO: 113 - protein, 28 aa, Artificial Sequence
  AceL-TerL N consensus sequence with increased thermostability
  misc_feature (2)..(2): Xaa is Leu or Val
  misc_feature (4)..(4): Xaa is Asp or Asn
  misc_feature (6)..(6): Xaa is Met, Ile, Ser or Leu
  misc_feature (7)..(7): Xaa is Ile or Val
  misc_feature (9)..(9): Xaa is Ile, Thr or Val
  misc_feature (10)..(10): Xaa is Leu, Glu, Gln, Lys or Ile
  misc_feature (11)..(11): Xaa is Asp or Lys
  misc_feature (12)..(12): Xaa is Asp, Gly, Glu or Val
  misc_feature (13)..(13): Xaa is Asp, Lys or Asn
  misc_feature (14)..(14): Xaa is Gly or Asn
  misc_feature (15)..(15): Xaa is Ile, Lys, Thr, Asn or Val
  misc_feature (16)..(16): Xaa is Ile, Thr, Pro, Ser or Glu
  misc_feature (17)..(17): Xaa is Lys or Glu
  misc_feature (18)..(18): Xaa is Ile, Thr, Val or Met
  misc_feature (19)..(19): Xaa is Ser or Arg
  misc_feature (20)..(20): Xaa is Met, Ile or Leu
  misc_feature (21)..(21): Xaa is Glu or Lys
  misc_feature (22)..(22): Xaa is Asp, Asn or Glu
  misc_feature (25)..(25): Xaa is Gln, Lys, Glu, Asp or Asn
  misc_feature (26)..(26): Xaa is Arg or Lys
  misc_feature (27)..(27): Xaa is Leu or Met
CXGXTXXEXXXXXXXXXXXXXXLYXXXA

SEQ ID NO: 114 - protein, 104 aa, Artificial Sequence
  AceL-TerL C consensus sequence
  MISC_FEATURE (2)..(2): Xaa is Tyr, Phe or Trp
  misc_feature (3)..(10): Xaa can be any naturally occurring amino acid
  MISC_FEATURE (11)..(11): Xaa is Ile, Val, Leu or Phe
  misc_feature (12)..(12): Xaa can be any naturally occurring amino acid
  MISC_FEATURE (13)..(13): Xaa is Thr or Ser
  misc_feature (15)..(15): Xaa can be any naturally occurring amino acid
  MISC_FEATURE (17)..(17): Xaa is Tyr, Phe or Trp
  misc_feature (18)..(19): Xaa can be any naturally occurring amino acid
  misc_feature (21)..(21): Xaa can be any naturally occurring amino acid
  MISC_FEATURE (23)..(23): Xaa is Ile, Val, Leu or Phe
  MISC_FEATURE (24)..(24): Xaa is Gln or Asn
  misc_feature (25)..(25): Xaa can be any naturally occurring amino acid
  MISC_FEATURE (26)..(26): Xaa is Ile, Val, Leu or Phe
  misc_feature (27)..(27): Xaa can be any naturally occurring amino acid
  MISC_FEATURE (28)..(28): Xaa is Lys, Arg or His
  misc_feature (29)..(30): Xaa can be any naturally occurring amino acid
  misc_feature (32)..(32): Xaa can be any naturally occurring amino acid
  MISC_FEATURE (33)..(33): Xaa is His or Trp
  MISC_FEATURE (34)..(34): Xaa is Ile, Val, Leu or Phe
  misc_feature (37)..(38): Xaa can be any naturally occurring amino acid
  MISC_FEATURE (39)..(39): Xaa is Gly or Lys
  misc_feature (40)..(40): Xaa can be any naturally occurring amino acid
  MISC_FEATURE (42)..(42): Xaa is Ile, Val, Leu or Phe
  MISC_FEATURE (43)..(43): Xaa is Lys, Arg or His
  MISC_FEATURE (44)..(44): Xaa is Cys or Thr
  misc_feature (46)..(46): Xaa can be any naturally occurring amino acid
  MISC_FEATURE (47)..(47): Xaa is Asp or Asn
  misc_feature (49)..(49): Xaa can be any naturally occurring amino acid
  MISC_FEATURE (50)..(50): Xaa is Ile, Val, Leu or Phe
  misc_feature (51)..(52): Xaa can be any naturally occurring amino acid
  MISC_FEATURE (53)..(53): Xaa is Asp or Glu
  misc_feature (54)..(54): Xaa can be any naturally occurring amino acid
  MISC_FEATURE (55)..(55): Xaa is Ile, Val or Thr
  misc_feature (56)..(56): Xaa can be any naturally occurring amino acid
  MISC_FEATURE (57)..(57): Xaa is Ala or Ser
  misc_feature (58)..(59): Xaa can be any naturally occurring amino acid
  MISC_FEATURE (60)..(60): Xaa is Ile, Val, Leu or Phe
  misc_feature (61)..(65): Xaa can be any naturally occurring amino acid
  MISC_FEATURE (66)..(66): Xaa is Ile, Val, Leu or Phe
  misc_feature (67)..(68): Xaa can be any naturally occurring amino acid
  misc_feature (70)..(70): Xaa can be any naturally occurring amino acid
  MISC_FEATURE (71)..(71): Xaa is Ile, Val, Leu or Phe
  misc_feature (72)..(72): Xaa can be any naturally occurring amino acid
  misc_feature (74)..(74): Xaa can be any naturally occurring amino acid
  misc_feature (76)..(76): Xaa can be any naturally occurring amino acid
  MISC_FEATURE (77)..(77): Xaa is Ile, Val, Leu or Phe
  misc_feature (78)..(78): Xaa can be any naturally occurring amino acid
  misc_feature (80)..(80): Xaa can be any naturally occurring amino acid
  MISC_FEATURE (81)..(81): Xaa is Ile, Val, Leu or Phe
  misc_feature (82)..(82): Xaa can be any naturally occurring amino acid
  MISC_FEATURE (86)..(86): Xaa is Ile, Val, Leu or Phe
  misc_feature (87)..(92): Xaa can be any naturally occurring amino acid
  MISC_FEATURE (93)..(93): Xaa is Asn or His
  misc_feature (94)..(94): Xaa can be any naturally occurring amino acid
  misc_feature (96)..(96): Xaa can be any naturally occurring amino acid
  MISC_FEATURE (97)..(97): Xaa is Thr or Ser
  misc_feature (99)..(99): Xaa can be any naturally occurring amino acid
  MISC_FEATURE (100)..(100): Xaa is Ile, Val, Leu or Phe
  MISC_FEATURE (101)..(101): Xaa is Ile, Val, Leu or Phe
  MISC_FEATURE (103)..(103): Xaa is His or Ser
MXXXXXXXXXXXXPXGXXXFXGXXXXXXXXYXXXIFXXXXEXXXSXXHXXXXXXXXXXXXXXXXXXXXKXXXYXEXXXEXXXLYDXXXXXXXXXYXXNXXXSXN

SEQ ID NO: 115 - protein, 103 aa, Artificial Sequence
  AceL-TerL C consensus sequence
  MISC_FEATURE (2)..(2): Xaa is Tyr, Phe or Trp
  misc_feature (3)..(9): Xaa can be any naturally occurring amino acid
  MISC_FEATURE (10)..(10): Xaa is Ile, Val, Leu or Phe
  misc_feature (11)..(11): Xaa can be any naturally occurring amino acid
  MISC_FEATURE (12)..(12): Xaa is Thr or Ser
  misc_feature (14)..(14): Xaa can be any naturally occurring amino acid
  MISC_FEATURE (16)..(16): Xaa is Tyr, Phe or Trp
  misc_feature (17)..(18): Xaa can be any naturally occurring amino acid
  misc_feature (20)..(20): Xaa can be any naturally occurring amino acid
  MISC_FEATURE (22)..(22): Xaa is Ile, Val, Leu or Phe
  MISC_FEATURE (23)..(23): Xaa is Gln or Asn
  misc_feature (24)..(24): Xaa can be any naturally occurring amino acid
  MISC_FEATURE (25)..(25): Xaa is Ile, Val, Leu or Phe
  misc_feature (26)..(26): Xaa can be any naturally occurring amino acid
  MISC_FEATURE (27)..(27): Xaa is Lys, Arg or His
  misc_feature (28)..(29): Xaa can be any naturally occurring amino acid
  misc_feature (31)..(31): Xaa can be any naturally occurring amino acid
  MISC_FEATURE (32)..(32): Xaa is His or Trp
  MISC_FEATURE (33)..(33): Xaa is Ile, Val, Leu or Phe
  misc_feature (36)..(37): Xaa can be any naturally occurring amino acid
  MISC_FEATURE (38)..(38): Xaa is Gly or Lys
  misc_feature (39)..(39): Xaa can be any naturally occurring amino acid
  MISC_FEATURE (41)..(41): Xaa is Ile, Val, Leu or Phe
  MISC_FEATURE (42)..(42): Xaa is Lys, Arg or His
  MISC_FEATURE (43)..(43): Xaa is Cys or Thr
  misc_feature (45)..(45): Xaa can be any naturally occurring amino acid
  MISC_FEATURE (46)..(46): Xaa is Asp or Asn
  misc_feature (48)..(48): Xaa can be any naturally occurring amino acid
  MISC_FEATURE (49)..(49): Xaa is Ile, Val, Leu or Phe
  misc_feature (50)..(51): Xaa can be any naturally occurring amino acid
  MISC_FEATURE (52)..(52): Xaa is Asp or Glu
  misc_feature (53)..(53): Xaa can be any naturally occurring amino acid
  MISC_FEATURE (54)..(54): Xaa is Ile, Val or Thr
  misc_feature (55)..(55): Xaa can be any naturally occurring amino acid
  MISC_FEATURE (56)..(56): Xaa is Ala or Ser
  misc_feature (57)..(58): Xaa can be any naturally occurring amino acid
  MISC_FEATURE (59)..(59): Xaa is Ile, Val, Leu or Phe
  misc_feature (60)..(64): Xaa can be any naturally occurring amino acid
  MISC_FEATURE (65)..(65): Xaa is Ile, Val, Leu or Phe
  misc_feature (66)..(67): Xaa can be any naturally occurring amino acid
  misc_feature (69)..(69): Xaa can be any naturally occurring amino acid
  MISC_FEATURE (70)..(70): Xaa is Ile, Val, Leu or Phe
  misc_feature (71)..(71): Xaa can be any naturally occurring amino acid
  misc_feature (73)..(73): Xaa can be any naturally occurring amino acid
  misc_feature (75)..(75): Xaa can be any naturally occurring amino acid
  MISC_FEATURE (76)..(76): Xaa is Ile, Val, Leu or Phe
  misc_feature (77)..(77): Xaa can be any naturally occurring amino acid
  misc_feature (79)..(79): Xaa can be any naturally occurring amino acid
  MISC_FEATURE (80)..(80): Xaa is Ile, Val, Leu or Phe
  misc_feature (81)..(81): Xaa can be any naturally occurring amino acid
  MISC_FEATURE (85)..(85): Xaa is Ile, Val, Leu or Phe
  misc_feature (86)..(91): Xaa can be any naturally occurring amino acid
  MISC_FEATURE (92)..(92): Xaa is Asn or His
  misc_feature (93)..(93): Xaa can be any naturally occurring amino acid
  misc_feature (95)..(95): Xaa can be any naturally occurring amino acid
  MISC_FEATURE (96)..(96): Xaa is Thr or Ser
  misc_feature (98)..(98): Xaa can be any naturally occurring amino acid
  MISC_FEATURE (99)..(100): Xaa is Ile, Val, Leu or Phe
  MISC_FEATURE (102)..(102): Xaa is His or Ser
MXXXXXXXXXXXPXGXXXFXGXXXXXXXXYXXXIFXXXXEXXXSXXHXXXXXXXXXXXXXXXXXXXXKXXXYXEXXXEXXXLYDXXXXXXXXXYXXNXXXSXN

SEQ ID NO: 116 - protein, 100 aa, Artificial Sequence
  AceL-TerL C consensus sequence
  misc_feature (1)..(6): Xaa can be any naturally occurring amino acid
  MISC_FEATURE (7)..(7): Xaa is Ile, Val, Leu or Phe
  misc_feature (8)..(8): Xaa can be any naturally occurring amino acid
  MISC_FEATURE (9)..(9): Xaa is Thr or Ser
  misc_feature (11)..(11): Xaa can be any naturally occurring amino acid
  MISC_FEATURE (13)..(13): Xaa is Tyr, Phe or Trp
  misc_feature (14)..(15): Xaa can be any naturally occurring amino acid
  misc_feature (17)..(17): Xaa can be any naturally occurring amino acid
  MISC_FEATURE (19)..(19): Xaa is Ile, Val, Leu or Phe
  MISC_FEATURE (20)..(20): Xaa is Gln or Asn
  misc_feature (21)..(21): Xaa can be any naturally occurring amino acid
  MISC_FEATURE (22)..(22): Xaa is Ile, Val, Leu or Phe
  misc_feature (23)..(23): Xaa can be any naturally occurring amino acid
  MISC_FEATURE (24)..(24): Xaa is Lys, Arg or His
  misc_feature (25)..(26): Xaa can be any naturally occurring amino acid
  misc_feature (28)..(28): Xaa can be any naturally occurring amino acid
  MISC_FEATURE (29)..(29): Xaa is His or Trp
  MISC_FEATURE (30)..(30): Xaa is Ile, Val, Leu or Phe
  misc_feature (33)..(34): Xaa can be any naturally occurring amino acid
  MISC_FEATURE (35)..(35): Xaa is Gly or Lys
  misc_feature (36)..(36): Xaa can be any naturally occurring amino acid
  MISC_FEATURE (38)..(38): Xaa is Ile, Val, Leu or Phe
  MISC_FEATURE (39)..(39): Xaa is Lys, Arg or His
  MISC_FEATURE (40)..(40): Xaa is Cys or Thr
  misc_feature (42)..(42): Xaa can be any naturally occurring amino acid
  MISC_FEATURE (43)..(43): Xaa is Asp or Asn
  misc_feature (45)..(45): Xaa can be any naturally occurring amino acid
  MISC_FEATURE (46)..(46): Xaa is Ile, Val, Leu or Phe
  misc_feature (47)..(48): Xaa can be any naturally occurring amino acid
  MISC_FEATURE (49)..(49): Xaa is Asp or Glu
  misc_feature (50)..(50): Xaa can be any naturally occurring amino acid
  MISC_FEATURE (51)..(51): Xaa is Ile, Val or Thr
  misc_feature (52)..(52): Xaa can be any naturally occurring amino acid
  MISC_FEATURE (53)..(53): Xaa is Ala or Ser
  misc_feature (54)..(55): Xaa can be any naturally occurring amino acid
  MISC_FEATURE (56)..(56): Xaa is Ile, Val, Leu or Phe
  misc_feature (57)..(61): Xaa can be any naturally occurring amino acid
  MISC_FEATURE (62)..(62): Xaa is Ile, Val, Leu or Phe
  misc_feature (63)..(64): Xaa can be any naturally occurring amino acid
  misc_feature (66)..(66): Xaa can be any naturally occurring amino acid
  MISC_FEATURE (67)..(67): Xaa is Ile, Val, Leu or Phe
  misc_feature (68)..(68): Xaa can be any naturally occurring amino acid
  misc_feature (70)..(70): Xaa can be any naturally occurring amino acid
  misc_feature (72)..(72): Xaa can be any naturally occurring amino acid
  MISC_FEATURE (73)..(73): Xaa is Ile, Val, Leu or Phe
  misc_feature (74)..(74): Xaa can be any naturally occurring amino acid
  misc_feature (76)..(76): Xaa can be any naturally occurring amino acid
  MISC_FEATURE (77)..(77): Xaa is Ile, Val, Leu or Phe
  misc_feature (78)..(78): Xaa can be any naturally occurring amino acid
  MISC_FEATURE (82)..(82): Xaa is Ile, Val, Leu or Phe
  misc_feature (83)..(88): Xaa can be any naturally occurring amino acid
  MISC_FEATURE (89)..(89): Xaa is Asn or His
  misc_feature (90)..(90): Xaa can be any naturally occurring amino acid
  misc_feature (92)..(92): Xaa can be any naturally occurring amino acid
  MISC_FEATURE (93)..(93): Xaa is Thr or Ser
  misc_feature (95)..(95): Xaa can be any naturally occurring amino acid
  MISC_FEATURE (96)..(97): Xaa is Ile, Val, Leu or Phe
  MISC_FEATURE (99)..(99): Xaa is His or Ser
XXXXXXXXXPXGXXXFXGXXXXXXXXYXXXIFXXXXEXXXSXXHXXXXXXXXXXXXXXXXXXXXKXXXYXEXXXEXXXLYDXXXXXXXXXYXXNXXXSXN

SEQ ID NO: 117 - protein, 101 aa, Artificial Sequence
  AceL-TerL C consensus sequence
  misc_feature (1)..(3): Xaa can be any naturally occurring amino acid
  MISC_FEATURE (4)..(4): Xaa is Ile, Val, Leu or Phe
  misc_feature (5)..(5): Xaa can be any naturally occurring amino acid
  MISC_FEATURE (6)..(6): Xaa is Thr or Ser
  misc_feature (8)..(8): Xaa can be any naturally occurring amino acid
  MISC_FEATURE (10)..(10): Xaa is Tyr, Phe or Trp
  misc_feature (11)..(12): Xaa can be any naturally occurring amino acid
  misc_feature (14)..(14): Xaa can be any naturally occurring amino acid
  MISC_FEATURE (16)..(16): Xaa is Ile, Val, Leu or Phe
  MISC_FEATURE (17)..(17): Xaa is Gln or Asn
  misc_feature (18)..(18): Xaa can be any naturally occurring amino acid
  MISC_FEATURE (19)..(19): Xaa is Ile, Val, Leu or Phe
  misc_feature (20)..(20): Xaa can be any naturally occurring amino acid
  MISC_FEATURE (21)..(21): Xaa is Lys, Arg or His
  misc_feature (22)..(23): Xaa can be any naturally occurring amino acid
  misc_feature (25)..(25): Xaa can be any naturally occurring amino acid
  MISC_FEATURE (26)..(26): Xaa is His or Trp
  MISC_FEATURE (27)..(27): Xaa is Ile, Val, Leu or Phe
  misc_feature (30)..(31): Xaa can be any naturally occurring amino acid
  MISC_FEATURE (32)..(32): Xaa is Gly or Lys
  misc_feature (33)..(33): Xaa can be any naturally occurring amino acid
  MISC_FEATURE (35)..(35): Xaa is Ile, Val, Leu or Phe
  MISC_FEATURE (36)..(36): Xaa is Lys, Arg or His
  MISC_FEATURE (37)..(37): Xaa is Cys or Thr
  misc_feature (39)..(39): Xaa can be any naturally occurring amino acid
  MISC_FEATURE (40)..(40): Xaa is Asp or Asn
  misc_feature (42)..(42): Xaa can be any naturally occurring amino acid
  MISC_FEATURE (43)..(43): Xaa is Ile, Val, Leu or Phe
  misc_feature (44)..(49): Xaa can be any naturally occurring amino acid
  MISC_FEATURE (50)..(50): Xaa is Asp or Glu
  misc_feature (51)..(51): Xaa can be any naturally occurring amino acid
  MISC_FEATURE (52)..(52): Xaa is Ile, Val or Thr
  misc_feature (53)..(53): Xaa can be any naturally occurring amino acid
  MISC_FEATURE (54)..(54): Xaa is Ala or Ser
  misc_feature (55)..(56): Xaa can be any naturally occurring amino acid
  MISC_FEATURE (57)..(57): Xaa is Ile, Val, Leu or Phe
  misc_feature (58)..(62): Xaa can be any naturally occurring amino acid
  MISC_FEATURE (63)..(63): Xaa is Ile, Val, Leu or Phe
  misc_feature (64)..(65): Xaa can be any naturally occurring amino acid
  misc_feature (67)..(67): Xaa can be any naturally occurring amino acid
  MISC_FEATURE (68)..(68): Xaa is Ile, Val, Leu or Phe
  misc_feature (69)..(69): Xaa can be any naturally occurring amino acid
  misc_feature (71)..(71): Xaa can be any naturally occurring amino acid
  misc_feature (73)..(73): Xaa can be any naturally occurring amino acid
  MISC_FEATURE (74)..(74): Xaa is Ile, Val, Leu or Phe
  misc_feature (75)..(75): Xaa can be any naturally occurring amino acid
  misc_feature (77)..(77): Xaa can be any naturally occurring amino acid
  MISC_FEATURE (78)..(78): Xaa is Ile, Val, Leu or Phe
  misc_feature (79)..(79): Xaa can be any naturally occurring amino acid
  MISC_FEATURE (83)..(83): Xaa is Ile, Val, Leu or Phe
  misc_feature (84)..(89): Xaa can be any naturally occurring amino acid
  MISC_FEATURE (90)..(90): Xaa is Asn or His
  misc_feature (91)..(91): Xaa can be any naturally occurring amino acid
  misc_feature (93)..(93): Xaa can be any naturally occurring amino acid
  MISC_FEATURE (94)..(94): Xaa is Thr or Ser
  misc_feature (96)..(96): Xaa can be any naturally occurring amino acid
  MISC_FEATURE (97)..(98): Xaa is Ile, Val, Leu or Phe
  MISC_FEATURE (100)..(100): Xaa is His or Ser
XXXXXXPXGXXXFXGXXXXXXXXYXXXIFXXXXEXXXSXXHXXXXXXXXXXXXXXXXXXXXXXXXKXXXYXEXXXEXXXLYDXXXXXXXXXYXXNXXXSXN

SEQ ID NO: 118 - protein, 107 aa, Artificial Sequence
  AceL-TerL C consensus sequence
  misc_feature (1)..(6): Xaa can be any naturally occurring amino acid
  MISC_FEATURE (7)..(7): Xaa is Ile, Val, Leu or Phe
  misc_feature (8)..(8): Xaa can be any naturally occurring amino acid
  MISC_FEATURE (9)..(9): Xaa is Thr or Ser
  misc_feature (11)..(11): Xaa can be any naturally occurring amino acid
  MISC_FEATURE (13)..(13): Xaa is Tyr, Phe or Trp
  misc_feature (14)..(15): Xaa can be any naturally occurring amino acid
  misc_feature (17)..(17): Xaa can be any naturally occurring amino acid
  MISC_FEATURE (19)..(19): Xaa is Ile, Val, Leu or Phe
  MISC_FEATURE (20)..(20): Xaa is Gln or Asn
  misc_feature (21)..(21): Xaa can be any naturally occurring amino acid
  MISC_FEATURE (22)..(22): Xaa is Ile, Val, Leu or Phe
  misc_feature (23)..(23): Xaa can be any naturally occurring amino acid
  MISC_FEATURE (24)..(24): Lys, Arg or His
  misc_feature (25)..(26): Xaa can be any naturally occurring amino acid
  misc_feature (28)..(28): Xaa can be any naturally occurring amino acid
  MISC_FEATURE (29)..(29): Xaa is His or Trp
  MISC_FEATURE (30)..(30): Xaa is Ile, Val, Leu or Phe
  misc_feature (33)..(34): Xaa can be any naturally occurring amino acid
  MISC_FEATURE (35)..(35): Xaa is Gly or Lys
  misc_feature (36)..(36): Xaa can be any naturally occurring amino acid
  MISC_FEATURE (38)..(38): Xaa is Ile, Val, Leu or Phe
  MISC_FEATURE (39)..(39): Lys, Arg or His
  MISC_FEATURE (40)..(40): Xaa is Cys or Thr
  misc_feature (42)..(42): Xaa can be any naturally occurring amino acid
  MISC_FEATURE (43)..(43): Xaa is Asp or Asn
  misc_feature (45)..(45): Xaa can be any naturally occurring amino acid
  MISC_FEATURE (46)..(46): Xaa is Ile, Val, Leu or Phe
  misc_feature (47)..(51): Xaa can be any naturally occurring amino acid
  MISC_FEATURE (52)..(52): Xaa is Asp or Glu
  misc_feature (53)..(53): Xaa can be any naturally occurring amino acid
  MISC_FEATURE (54)..(54): Xaa is Ile, Val or Thr
  misc_feature (55)..(55): Xaa can be any naturally occurring amino acid
  MISC_FEATURE (56)..(56): Xaa is Ala or Ser
  misc_feature (57)..(58): Xaa can be any naturally occurring amino acid
  MISC_FEATURE (59)..(59): Xaa is Ile, Val, Leu or Phe
  misc_feature (60)..(64): Xaa can be any naturally occurring amino acid
  MISC_FEATURE (65)..(65): Xaa is Ile, Val, Leu or Phe
  misc_feature (66)..(67): Xaa can be any naturally occurring amino acid
  misc_feature (69)..(73): Xaa can be any naturally occurring amino acid
  MISC_FEATURE (74)..(74): Xaa is Ile, Val, Leu or Phe
  misc_feature (75)..(75): Xaa can be any naturally occurring amino acid
  misc_feature (77)..(77): Xaa can be any naturally occurring amino acid
  misc_feature (79)..(79): Xaa can be any naturally occurring amino acid
  MISC_FEATURE (80)..(80): Xaa is Ile, Val, Leu or Phe
  misc_feature (81)..(81): Xaa can be any naturally occurring amino acid
  misc_feature (83)..(83): Xaa can be any naturally occurring amino acid
  MISC_FEATURE (84)..(84): Xaa is Ile, Val, Leu or Phe
  misc_feature (85)..(85): Xaa can be any naturally occurring amino acid
  MISC_FEATURE (89)..(89): Xaa is Ile, Val, Leu or Phe
  misc_feature (90)..(95): Xaa can be any naturally occurring amino acid
  MISC_FEATURE (96)..(96): Xaa is Asn or His
  misc_feature (97)..(97): Xaa can be any naturally occurring amino acid
  misc_feature (99)..(99): Xaa can be any naturally occurring amino acid
  MISC_FEATURE (100)..(100): Xaa is Thr or Ser
  misc_feature (102)..(102): Xaa can be any naturally occurring amino acid
  MISC_FEATURE (103)..(104): Xaa is Ile, Val, Leu or Phe
  MISC_FEATURE (106)..(106): Xaa is His or Ser
XXXXXXXXXPXGXXXFXGXXXXXXXXYXXXIFXXXXEXXXSXXHXXXXXXXXXXXXXXXXXXXXXXXKXXXXXXXYXEXXXEXXXLYDXXXXXXXXXYXXNXXXSXN

SEQ ID NO: 119 - protein, 111 aa, Artificial Sequence
  AceL-TerL C consensus sequence
  MISC_FEATURE (2)..(2): Xaa is Tyr, Phe or Trp
  misc_feature (3)..(10): Xaa can be any naturally occurring amino acid
  MISC_FEATURE (11)..(11): Xaa is Ile, Val, Leu or Phe
  misc_feature (12)..(12): Xaa can be any naturally occurring amino acid
  MISC_FEATURE (13)..(13): Xaa is Thr or Ser
  misc_feature (15)..(15): Xaa can be any naturally occurring amino acid
  MISC_FEATURE (17)..(17): Xaa is Tyr, Phe or Trp
  misc_feature (18)..(19): Xaa can be any naturally occurring amino acid
  misc_feature (21)..(21): Xaa can be any naturally occurring amino acid
  MISC_FEATURE (23)..(23): Xaa is Ile, Val, Leu or Phe
  MISC_FEATURE (24)..(24): Xaa is Gln or Asn
  misc_feature (25)..(25): Xaa can be any naturally occurring amino acid
  MISC_FEATURE (26)..(26): Xaa is Ile, Val, Leu or Phe
  misc_feature (27)..(27): Xaa can be any naturally occurring amino acid
  MISC_FEATURE (28)..(28): Xaa is Lys, Arg or His
  misc_feature (29)..(30): Xaa can be any naturally occurring amino acid
  misc_feature (32)..(32): Xaa can be any naturally occurring amino acid
  MISC_FEATURE (33)..(33): Xaa is  His or Trp
  MISC_FEATURE (34)..(34): Xaa is Ile, Val, Leu or Phe
  misc_feature (37)..(38): Xaa can be any naturally occurring amino acid
  MISC_FEATURE (39)..(39): Xaa is Gly or Lys
  misc_feature (40)..(40): Xaa can be any naturally occurring amino acid
  MISC_FEATURE (42)..(42): Xaa is Ile, Val, Leu or Phe
  MISC_FEATURE (43)..(43): Xaa is Lys, Arg or His
  MISC_FEATURE (44)..(44): Xaa is Cys or Thr
  misc_feature (46)..(46): Xaa can be any naturally occurring amino acid
  MISC_FEATURE (47)..(47): Xaa is Asp or Asn
  misc_feature (49)..(49): Xaa can be any naturally occurring amino acid
  MISC_FEATURE (50)..(50): Xaa is Ile, Val, Leu or Phe
  misc_feature (51)..(55): Xaa can be any naturally occurring amino acid
  MISC_FEATURE (56)..(56): Xaa is Asp or Glu
  misc_feature (57)..(57): Xaa can be any naturally occurring amino acid
  MISC_FEATURE (58)..(58): Xaa is Ile, Val or Thr
  misc_feature (59)..(59): Xaa can be any naturally occurring amino acid
  MISC_FEATURE (60)..(60): Xaa is Ala or Ser
  misc_feature (61)..(62): Xaa can be any naturally occurring amino acid
  MISC_FEATURE (63)..(63): Xaa is Ile, Val, Leu or Phe
  misc_feature (64)..(68): Xaa can be any naturally occurring amino acid
  MISC_FEATURE (69)..(69): Xaa is Ile, Val, Leu or Phe
  misc_feature (70)..(71): Xaa can be any naturally occurring amino acid
  misc_feature (73)..(77): Xaa can be any naturally occurring amino acid
  MISC_FEATURE (78)..(78): Xaa is Ile, Val, Leu or Phe
  misc_feature (79)..(79): Xaa can be any naturally occurring amino acid
  misc_feature (81)..(81): Xaa can be any naturally occurring amino acid
  misc_feature (83)..(83): Xaa can be any naturally occurring amino acid
  MISC_FEATURE (84)..(84): Xaa is Ile, Val, Leu or Phe
  misc_feature (85)..(85): Xaa can be any naturally occurring amino acid
  misc_feature (87)..(87): Xaa can be any naturally occurring amino acid
  MISC_FEATURE (88)..(88): Xaa is Ile, Val, Leu or Phe
  misc_feature (89)..(89): Xaa can be any naturally occurring amino acid
  MISC_FEATURE (93)..(93): Xaa is Ile, Val, Leu or Phe
  misc_feature (94)..(99): Xaa can be any naturally occurring amino acid
  MISC_FEATURE (100)..(100): Xaa is Asn or His
  misc_feature (101)..(101): Xaa can be any naturally occurring amino acid
  misc_feature (103)..(103): Xaa can be any naturally occurring amino acid
  MISC_FEATURE (104)..(104): Xaa is Thr or Ser
  misc_feature (106)..(106): Xaa can be any naturally occurring amino acid
  MISC_FEATURE (107)..(108): Xaa is Ile, Val, Leu or Phe
  MISC_FEATURE (110)..(110): Xaa is His or Ser
MXXXXXXXXXXXXPXGXXXFXGXXXXXXXXYXXXIFXXXXEXXXSXXHXXXXXXXXXXXXXXXXXXXXXXXKXXXXXXXYXEXXXEXXXLYDXXXXXXXXXYXXNXXXSXN

SEQ ID NO: 120 - protein, 108 aa, Artificial Sequence
  AceL-TerL C consensus sequence
  misc_feature (1)..(6): Xaa can be any naturally occurring amino acid
  MISC_FEATURE (7)..(7): Xaa is Tyr, Phe or Trp
  misc_feature (8)..(14): Xaa can be any naturally occurring amino acid
  MISC_FEATURE (15)..(15): Xaa is Ile, Val, Leu or Phe
  misc_feature (16)..(16): Xaa can be any naturally occurring amino acid
  MISC_FEATURE (17)..(17): Xaa is Thr or Ser
  misc_feature (19)..(19): Xaa can be any naturally occurring amino acid
  MISC_FEATURE (21)..(21): Xaa is Tyr, Phe or Trp
  misc_feature (22)..(23): Xaa can be any naturally occurring amino acid
  misc_feature (25)..(25): Xaa can be any naturally occurring amino acid
  MISC_FEATURE (27)..(27): Xaa is Ile, Val, Leu or Phe
  MISC_FEATURE (28)..(28): Xaa is Gln or Asn
  misc_feature (29)..(29): Xaa can be any naturally occurring amino acid
  MISC_FEATURE (30)..(30): Xaa is Ile, Val, Leu or Phe
  misc_feature (31)..(31): Xaa can be any naturally occurring amino acid
  MISC_FEATURE (32)..(32): Xaa is Lys, Arg or His
  misc_feature (33)..(34): Xaa can be any naturally occurring amino acid
  misc_feature (36)..(36): Xaa can be any naturally occurring amino acid
  MISC_FEATURE (37)..(37): Xaa is His or Trp
  MISC_FEATURE (38)..(38): Xaa is Ile, Val, Leu or Phe
  misc_feature (41)..(42): Xaa can be any naturally occurring amino acid
  MISC_FEATURE (43)..(43): Xaa is Gly or Lys
  misc_feature (44)..(44): Xaa can be any naturally occurring amino acid
  MISC_FEATURE (46)..(46): Xaa is Ile, Val, Leu or Phe
  MISC_FEATURE (47)..(47): Xaa is Lys, Arg or His
  MISC_FEATURE (48)..(48): Xaa is Cys or Thr
  misc_feature (50)..(50): Xaa can be any naturally occurring amino acid
  MISC_FEATURE (51)..(51): Xaa is Asp or Asn
  MISC_FEATURE (53)..(53): Xaa is Ile, Val, Leu or Phe
  misc_feature (54)..(55): Xaa can be any naturally occurring amino acid
  MISC_FEATURE (56)..(56): Xaa is Asp or Glu
  misc_feature (57)..(57): Xaa can be any naturally occurring amino acid
  MISC_FEATURE (58)..(58): Xaa is Ile, Val or Thr
  misc_feature (59)..(59): Xaa can be any naturally occurring amino acid
  MISC_FEATURE (60)..(60): Xaa is Ala or Ser
  misc_feature (61)..(67): Xaa can be any naturally occurring amino acid
  MISC_FEATURE (68)..(68): Xaa is Ile, Val, Leu or Phe
  MISC_FEATURE (69)..(69): Xaa is Ile, Val, Leu or Phe
  misc_feature (70)..(72): Xaa can be any naturally occurring amino acid
  MISC_FEATURE (74)..(74): Xaa is Ile, Val, Leu or Phe
  misc_feature (75)..(76): Xaa can be any naturally occurring amino acid
  misc_feature (78)..(78): Xaa can be any naturally occurring amino acid
  MISC_FEATURE (80)..(80): Xaa is Ile, Val, Leu or Phe
  misc_feature (81)..(82): Xaa can be any naturally occurring amino acid
  MISC_FEATURE (84)..(84): Xaa is Ile, Val, Leu or Phe
  misc_feature (85)..(86): Xaa can be any naturally occurring amino acid
  MISC_FEATURE (89)..(89): Xaa is Ile, Val, Leu or Phe
  misc_feature (90)..(95): Xaa can be any naturally occurring amino acid
  MISC_FEATURE (96)..(96): Xaa is Asn or His
  misc_feature (97)..(98): Xaa can be any naturally occurring amino acid
  MISC_FEATURE (100)..(100): Xaa is Thr or Ser
  misc_feature (101)..(101): Xaa can be any naturally occurring amino acid
  MISC_FEATURE (103)..(104): Xaa is Ile, Val, Leu or Phe
  misc_feature (105)..(105): Xaa can be any naturally occurring amino acid
  MISC_FEATURE (106)..(106): Xaa is His or Ser
  misc_feature (107)..(107): Xaa can be any naturally occurring amino acid
XXXXXXXXXXXXXXXXXPXGXXXFXGXXXXXXXXYXXXIFXXXXEXXXSXXHXXXXXXXXXXXXXXXXXXXXKXXXYXEXXXEXXXLYDXXXXXXXXXYXXNXXXSXN

SEQ ID NO: 121 - protein, 104 aa, Artificial Sequence
  AceL-TerL C consensus sequence
  MISC_FEATURE (2)..(2): Xaa is Phe, Tyr or Ile
  MISC_FEATURE (3)..(3): Xaa is Lys, Arg or Ser
  MISC_FEATURE (4)..(4): Xaa is Leu, Ile, Thr, Val, Lys or Arg
  MISC_FEATURE (5)..(5): Xaa is Asn, Met or Thr
  MISC_FEATURE (6)..(6): Xaa is Thr, Ser or Phe
  MISC_FEATURE (7)..(7): Xaa is Lys, Asn, Thr, Ser or Asp
  MISC_FEATURE (8)..(8): Xaa is Asn, Thr, Ser, Met or Lys
  MISC_FEATURE (9)..(9): Xaa is lle or Tyr
  MISC_FEATURE (10)..(10): Xaa is Lys, Glu, Arg or Thr
  MISC_FEATURE (11)..(11): Xaa is Val or Ile
  MISC_FEATURE (12)..(12): Xaa is Leu or Lys
  MISC_FEATURE (13)..(13): Xaa is Thr or Ser
  MISC_FEATURE (15)..(15): Xaa is Ser, Asn, Asp, Thr or Arg
  MISC_FEATURE (17)..(17): Xaa is Phe or Tyr
  MISC_FEATURE (18)..(18): Xaa is Lys, Ser, Gln, Val or Glu
  MISC_FEATURE (19)..(19): Xaa is Ser, Asn, Asp, Lys, Phe or Tyr
  MISC_FEATURE (21)..(21): Xaa is Ser, Asn Ile, Ala or Asp
  MISC_FEATURE (23)..(23): Xaa is Ile or Val
  MISC_FEATURE (24)..(24): Xaa is Gln or Asn
  MISC_FEATURE (25)..(25): Xaa is Lys, Thr or Arg
  MISC_FEATURE (26)..(26): Xaa is Val or Ile
  MISC_FEATURE (27)..(27): Xaa is Tyr, Glu, Lys, Phe, Ser, Arg or Pro
  MISC_FEATURE (28)..(28): Xaa is Lys, Arg or His
  MISC_FEATURE (29)..(29): Xaa is Pro, Asn, Asp, Lys or Ser
  MISC_FEATURE (30)..(30): Xaa is Phe, Leu, Gln, Lys, Val, Met or Tyr
  MISC_FEATURE (32)..(32): Xaa is His, Gln, Arg or Ser
  MISC_FEATURE (33)..(33): Xaa is His or Trp
  MISC_FEATURE (34)..(34): Xaa is Ile, Phe or Leu
  MISC_FEATURE (37)..(37): Xaa is Asp, Glu, Gly or Ser
  MISC_FEATURE (38)..(38): Xaa is Asp, Gly or Ser
  MISC_FEATURE (39)..(39): Xaa is Gly or Lys
  MISC_FEATURE (40)..(40): Xaa is Ser, Thr, Ala or Ile
  MISC_FEATURE (42)..(42): Xaa is Ile or Leu
  MISC_FEATURE (43)..(43): Xaa is Lyr or Arg
  MISC_FEATURE (44)..(44): Xaa is Cys or Thr
  MISC_FEATURE (46)..(46): Xaa is Asp, Leu, Ile or Phe
  MISC_FEATURE (47)..(47): Xaa is Asn or Asp
  MISC_FEATURE (49)..(49): Xaa is Ser, Pro or Arg
  MISC_FEATURE (50)..(50): Xaa is Phe or Leu
  MISC_FEATURE (51)..(51): Xaa is Gly, Asp or Tyr
  MISC_FEATURE (52)..(52): Xaa is Lys, Ser, Glu or Gly
  MISC_FEATURE (53)..(53): Xaa is Asp or Glu
  MISC_FEATURE (54)..(54): Xaa is Lys, Glu, Gln or Ile
  MISC_FEATURE (55)..(55): Xaa is Ile, Val or Thr
  MISC_FEATURE (56)..(56): Xaa is Lys or Leu
  MISC_FEATURE (57)..(57): Xaa is Ala or Ser
  MISC_FEATURE (58)..(58): Xaa is Ser, Arg, His or Tyr
  MISC_FEATURE (59)..(59): Xaa is Thr, Asp, Ser, Glu, Met or Asn
  MISC_FEATURE (60)..(60): Xaa is Ile, Val or Leu
  MISC_FEATURE (61)..(61): Xaa is Lys, Trp, Arg, Asn or Cys
  MISC_FEATURE (62)..(62): Xaa is Val, Leu, Pro, Thr, Arg or Ile
  MISC_FEATURE (63)..(63): Xaa is Gly, Asp or Ser
  MISC_FEATURE (64)..(64): Xaa is Asp, Ser, Glu or Ala
  MISC_FEATURE (65)..(65): Xaa is Tyr, Ile, Asp, Phe, Leu, Glu or Lys
  MISC_FEATURE (66)..(66): Xaa is Leu, Ile or Val
  MISC_FEATURE (67)..(67): Xaa is Gln, Asn or Leu
  MISC_FEATURE (68)..(68): Xaa is Gly, Ser, Asn, Glu, His or Lys
  MISC_FEATURE (70)..(70): Xaa is Lys, Asn, Glu or Gln
  MISC_FEATURE (71)..(71): Xaa is Val or Ile
  MISC_FEATURE (72)..(72): Xaa is Leu, Val or Thr
  MISC_FEATURE (74)..(74): Xaa is Asn, Ala, Ser or Val
  MISC_FEATURE (76)..(76): Xaa is Ile, Leu, Asp or Val
  MISC_FEATURE (77)..(77): Xaa is Val or Ile
  MISC_FEATURE (78)..(78): Xaa is Glu, Asn or Ala
  MISC_FEATURE (80)..(80): Xaa is Gly, Asn, Lys, Asp, Pro, Gln or Glu
  MISC_FEATURE (81)..(81): Xaa is Ile or Val
  MISC_FEATURE (82)..(82): Xaa is Tyr, Phe, Asp or Thr
  MISC_FEATURE (86)..(86): Xaa is Tyr, Phe, Asp or Thr
  MISC_FEATURE (87)..(87): Xaa is Leu, Ile or Met
  MISC_FEATURE (88)..(88): Xaa is Asn, Asp or Glu
  MISC_FEATURE (89)..(89): Xaa is Val or Ser
  MISC_FEATURE (90)..(90): Xaa is Gly, Glu or Asp
  MISC_FEATURE (91)..(91): Xaa is Glu, Lys, Gly or Asp
  MISC_FEATURE (92)..(92): Xaa is Asp, Glu, Gly or Asn
  MISC_FEATURE (93)..(93): Xaa is Asn, Ser or His
  MISC_FEATURE (94)..(94): Xaa is Leu or Asn
  MISC_FEATURE (96)..(96): Xaa is Tyr, Ile, Asn or Phe
  MISC_FEATURE (97)..(97): Xaa is Thr or Ser
  MISC_FEATURE (99)..(99): Xaa is Gly, Asp, Lys, Asn, Asp or Glu
  MISC_FEATURE (100)..(100): Xaa is Ile, Val or Leu
  MISC_FEATURE (101)..(101): Xaa is Val or Ile
MXXXXXXXXXXXXPXGXXXFXGXXXXXXXXYXXXIFXXXXEXXXSXXHXXXXXXXXXXXXXXXXXXXXKXXXYXEXXXEXXXLYDXXXXXXXXXYXXNXXXSHN

SEQ ID NO: 122 - protein, 103 aa, Artificial Sequence
  AceL-TerL C consensus sequence
  MISC_FEATURE (2)..(2): Xaa is Phe, Tyr or Ile
  MISC_FEATURE (3)..(3): Xaa is Lys, Arg or Ser
  MISC_FEATURE (4)..(4): Xaa is Leu, Ile, Thr, Val, Lys or Arg
  MISC_FEATURE (5)..(5): Xaa is Asn, Met or Thr
  MISC_FEATURE (6)..(6): Xaa is Lys, Asn, Thr, Ser or Asp
  MISC_FEATURE (7)..(7): Xaa is Asn, Thr, Ser, Met or Lys
  MISC_FEATURE (8)..(8): Xaa is lle or Tyr
  MISC_FEATURE (9)..(9): Xaa is Lys, Glu, Arg or Thr
  MISC_FEATURE (10)..(10): Xaa is Val or Ile
  MISC_FEATURE (11)..(11): Xaa is Leu or Lys
  MISC_FEATURE (12)..(12): Xaa is Thr or Ser
  MISC_FEATURE (14)..(14): Xaa is Ser, Asn, Asp, Thr or Arg
  MISC_FEATURE (16)..(16): Xaa is Phe or Tyr
  MISC_FEATURE (17)..(17): Xaa is Lys, Ser, Gln, Val or Glu
  MISC_FEATURE (18)..(18): Xaa is Ser, Asn, Asp, Lys, Phe or Tyr
  MISC_FEATURE (20)..(20): Xaa is Ser, Asn Ile, Ala or Asp
  MISC_FEATURE (22)..(22): Xaa is Ile or Val
  MISC_FEATURE (23)..(23): Xaa is Gln or Asn
  MISC_FEATURE (24)..(24): Xaa is Lys, Thr or Arg
  MISC_FEATURE (25)..(25): Xaa is Val or Ile
  MISC_FEATURE (26)..(26): Xaa is Tyr, Glu, Lys, Phe, Ser, Arg or Pro
  MISC_FEATURE (27)..(27): Xaa is Lys, Arg or His
  MISC_FEATURE (28)..(28): Xaa is Pro, Asn, Asp, Lys or Ser
  MISC_FEATURE (29)..(29): Xaa is Phe, Leu, Gln, Lys, Val, Met or Tyr
  MISC_FEATURE (31)..(31): Xaa is His, Gln, Arg or Ser
  MISC_FEATURE (32)..(32): Xaa is His or Trp
  MISC_FEATURE (33)..(33): Xaa is Ile, Phe or Leu
  MISC_FEATURE (36)..(36): Xaa is Asp, Glu, Gly or Ser
  MISC_FEATURE (37)..(37): Xaa is Asp, Gly or Ser
  MISC_FEATURE (38)..(38): Xaa is Gly or Lys
  MISC_FEATURE (39)..(39): Xaa is Ser, Thr, Ala or Ile
  MISC_FEATURE (41)..(41): Xaa is Ile or Leu
  MISC_FEATURE (42)..(42): Xaa is Lyr or Arg
  MISC_FEATURE (43)..(43): Xaa is Cys or Thr
  MISC_FEATURE (45)..(45): Xaa is Asp, Leu, Ile or Phe
  MISC_FEATURE (46)..(46): Xaa is Asn or Asp
  MISC_FEATURE (48)..(48): Xaa is Ser, Pro or Arg
  MISC_FEATURE (49)..(49): Xaa is Phe or Leu
  MISC_FEATURE (50)..(50): Xaa is Gly, Asp or Tyr
  MISC_FEATURE (51)..(51): Xaa is Lys, Ser, Glu or Gly
  MISC_FEATURE (52)..(52): Xaa is Asp or Glu
  MISC_FEATURE (53)..(53): Xaa is Lys, Glu, Gln or Ile
  MISC_FEATURE (54)..(54): Xaa is Ile, Val or Thr
  MISC_FEATURE (55)..(55): Xaa is Lys or Leu
  MISC_FEATURE (56)..(56): Xaa is Ala or Ser
  MISC_FEATURE (57)..(57): Xaa is Ser, Arg, His or Tyr
  MISC_FEATURE (58)..(58): Xaa is Thr, Asp, Ser, Glu, Met or Asn
  MISC_FEATURE (59)..(59): Xaa is Ile, Val or Leu
  MISC_FEATURE (60)..(60): Xaa is Lys, Trp, Arg, Asn or Cys
  MISC_FEATURE (61)..(61): Xaa is Val, Leu, Pro, Thr, Arg or Ile
  MISC_FEATURE (62)..(62): Xaa is Gly, Asp or Ser
  MISC_FEATURE (63)..(63): Xaa is Asp, Ser, Glu or Ala
  MISC_FEATURE (64)..(64): Xaa is Tyr, Ile, Asp, Phe, Leu, Glu or Lys
  MISC_FEATURE (65)..(65): Xaa is Leu, Ile or Val
  MISC_FEATURE (66)..(66): Xaa is Gln, Asn or Leu
  MISC_FEATURE (67)..(67): Xaa is Gly, Ser, Asn, Glu, His or Lys
  MISC_FEATURE (69)..(69): Xaa is Lys, Asn, Glu or Gln
  MISC_FEATURE (70)..(70): Xaa is Val or Ile
  MISC_FEATURE (71)..(71): Xaa is Leu, Val or Thr
  MISC_FEATURE (73)..(73): Xaa is Asn, Ala, Ser or Val
  MISC_FEATURE (75)..(75): Xaa is Ile, Leu, Asp or Val
  MISC_FEATURE (76)..(76): Xaa is Val or Ile
  MISC_FEATURE (77)..(77): Xaa is Glu, Asn or Ala
  MISC_FEATURE (79)..(79): Xaa is Gly, Asn, Lys, Asp, Pro, Gln or Glu
  MISC_FEATURE (80)..(80): Xaa is Ile or Val
  MISC_FEATURE (81)..(81): Xaa is Tyr, Phe, Asp or Thr
  MISC_FEATURE (85)..(85): Xaa is Tyr, Phe, Asp or Thr
  MISC_FEATURE (86)..(86): Xaa is Leu, Ile or Met
  MISC_FEATURE (87)..(87): Xaa is Asn, Asp or Glu
  MISC_FEATURE (88)..(88): Xaa is Val or Ser
  MISC_FEATURE (89)..(89): Xaa is Gly, Glu or Asp
  MISC_FEATURE (90)..(90): Xaa is Glu, Lys, Gly or Asp
  MISC_FEATURE (91)..(91): Xaa is Asp, Glu, Gly or Asn
  MISC_FEATURE (92)..(92): Xaa is Asn, Ser or His
  MISC_FEATURE (93)..(93): Xaa is Leu or Asn
  MISC_FEATURE (95)..(95): Xaa is Tyr, Ile, Asn or Phe
  MISC_FEATURE (96)..(96): Xaa is Thr or Ser
  MISC_FEATURE (98)..(98): Xaa is Gly, Asp, Lys, Asn, Asp or Glu
  MISC_FEATURE (99)..(99): Xaa is Ile, Val or Leu
  MISC_FEATURE (100)..(100): Xaa is Val or Ile
MXXXXXXXXXXXPXGXXXFXGXXXXXXXXYXXXIFXXXXEXXXSXXHXXXXXXXXXXXXXXXXXXXXKXXXYXEXXXEXXXLYDXXXXXXXXXYXXNXXXSHN

SEQ ID NO: 123 - protein, 104 aa, Artificial Sequence
  AceL-TerL C consensus sequence with increased thermostability
  MISC_FEATURE (2)..(2): Xaa is Phe or Tyr
  MISC_FEATURE (3)..(3): Xaa is Lys or Arg
  MISC_FEATURE (4)..(4): Xaa is Leu or Thr
  MISC_FEATURE (5)..(5): Xaa is Asn or Met
  MISC_FEATURE (6)..(6): Xaa is Thr, Ser or Phe
  MISC_FEATURE (7)..(7): Xaa is Lys, Asn, Thr or Asp
  MISC_FEATURE (8)..(8): Xaa is Asn, Thr or Lys
  MISC_FEATURE (9)..(9): Xaa is lle or Tyr
  MISC_FEATURE (10)..(10): Xaa is Lys or Glu
  MISC_FEATURE (11)..(11): Xaa is Val or Ile
  MISC_FEATURE (12)..(12): Xaa is Leu or Lys
  MISC_FEATURE (13)..(13): Xaa is Thr or Ser
  MISC_FEATURE (15)..(15): Xaa is Ser, Asn, Asp or Arg
  MISC_FEATURE (17)..(17): Xaa is Phe or Tyr
  MISC_FEATURE (18)..(18): Xaa is Lys, Ser, Val or Glu
  MISC_FEATURE (19)..(19): Xaa is Ser, Asn, or Lys
  MISC_FEATURE (21)..(21): Xaa is Ser, Asn or Asp
  MISC_FEATURE (23)..(23): Xaa is Ile or Val
  MISC_FEATURE (24)..(24): Xaa is Gln or Asn
  MISC_FEATURE (25)..(25): Xaa is Lys or Arg
  MISC_FEATURE (26)..(26): Xaa is Val or Ile
  MISC_FEATURE (27)..(27): Xaa is Tyr, Lys, Ser or Arg
  MISC_FEATURE (28)..(28): Xaa is Lys, Arg or His
  MISC_FEATURE (29)..(29): Xaa is Pro, Asp or Ser
  MISC_FEATURE (30)..(30): Xaa is Phe, Leu, Lys or Met
  MISC_FEATURE (32)..(32): Xaa is His, Gln, Arg or Ser
  MISC_FEATURE (33)..(33): Xaa is His or Trp
  MISC_FEATURE (34)..(34): Xaa is Ile, Phe or Leu
  MISC_FEATURE (37)..(37): Xaa is Asp, Glu or Ser
  MISC_FEATURE (38)..(38): Xaa is Asp or Ser
  MISC_FEATURE (39)..(39): Xaa is Gly or Lys
  MISC_FEATURE (40)..(40): Xaa is Ser, Thr or Ile
  MISC_FEATURE (42)..(42): Xaa is Ile or Leu
  MISC_FEATURE (43)..(43): Xaa is Lyr or Arg
  MISC_FEATURE (44)..(44): Xaa is Cys or Thr
  MISC_FEATURE (46)..(46): Xaa is Asp, Leu or Ile
  MISC_FEATURE (47)..(47): Xaa is Asn or Asp
  MISC_FEATURE (49)..(49): Xaa is Ser, Pro or Arg
  MISC_FEATURE (50)..(50): Xaa is Phe or Leu
  MISC_FEATURE (51)..(51): Xaa is Gly or Tyr
  MISC_FEATURE (52)..(52): Xaa is Lys, Glu or Gly
  MISC_FEATURE (53)..(53): Xaa is Asp or Glu
  MISC_FEATURE (54)..(54): Xaa is Lys, Glu, Gln or Ile
  misc_feature (55)..(55): Xaa can be any naturally occurring amino acid
  MISC_FEATURE (56)..(56): Xaa is Lys or Leu
  MISC_FEATURE (57)..(57): Xaa is Ala or Ser
  MISC_FEATURE (58)..(58): Xaa is Ser, Arg or Tyr
  MISC_FEATURE (59)..(59): Xaa is Thr, Asp, Ser, Glu, Met or Asn
  MISC_FEATURE (60)..(60): Xaa is Ile, Val or Leu
  MISC_FEATURE (61)..(61): Xaa is Lys, Trp or Arg
  MISC_FEATURE (62)..(62): Xaa is Val, Arg or Ile
  MISC_FEATURE (63)..(63): Xaa is Gly or Asp
  MISC_FEATURE (64)..(64): Xaa is Asp or Ser
  MISC_FEATURE (65)..(65): Xaa is Tyr, Ile, Asp, Phe, Leu or Lys
  MISC_FEATURE (66)..(66): Xaa is Leu or Val
  MISC_FEATURE (67)..(67): Xaa is Gln, Asn or Leu
  MISC_FEATURE (68)..(68): Xaa is Gly or Ser
  MISC_FEATURE (70)..(70): Xaa is Lys or Asn
  MISC_FEATURE (71)..(71): Xaa is Val or Ile
  MISC_FEATURE (72)..(72): Xaa is Leu, Val or Thr
  MISC_FEATURE (74)..(74): Xaa is Asn or Ala
  MISC_FEATURE (76)..(76): Xaa is Ile, Leu or Asp
  MISC_FEATURE (77)..(77): Xaa is Val or Ile
  MISC_FEATURE (78)..(78): Xaa is Glu or Asn
  MISC_FEATURE (80)..(80): Xaa is Gly, Asn, Lys, Asp or Pro
  MISC_FEATURE (81)..(81): Xaa is Ile or Val
  MISC_FEATURE (82)..(82): Xaa is Tyr, Phe, Asp or Thr
  MISC_FEATURE (86)..(86): Xaa is Lys, Pro or Ile
  MISC_FEATURE (87)..(87): Xaa is Leu or Ile
  MISC_FEATURE (88)..(88): Xaa is Asn or Asp
  MISC_FEATURE (89)..(89): Xaa is Val or Ser
  MISC_FEATURE (90)..(90): Xaa is Gly or Glu
  MISC_FEATURE (91)..(91): Xaa is Glu, Lys or Gly
  MISC_FEATURE (92)..(92): Xaa is Asp, Glu, Gly or Asn
  MISC_FEATURE (92)..(92): Xaa is Asp, Glu or Gly
  MISC_FEATURE (93)..(93): Xaa is Asn, Ser or His
  MISC_FEATURE (94)..(94): Xaa is Leu or Asn
  MISC_FEATURE (96)..(96): Xaa is Tyr or Ile
  MISC_FEATURE (97)..(97): Xaa is Thr or Ser
  MISC_FEATURE (99)..(99): Xaa is Gly, Asp or Asn
  MISC_FEATURE (100)..(100): Xaa is Ile, Val or Leu
  MISC_FEATURE (101)..(101): Xaa is Val or Ile
MXXXXXXXXXXXXPXGXXXFXGXXXXXXXXYXXXIFXXXXEXXXSXXHXIXXXXXXXXXXXXXXXXXXKXXXYXEXXXEXXXLYDXXXXXXXXXYXXNXXXSHN

SEQ ID NO: 124 - protein, 103 aa, Artificial Sequence
  AceL-TerL C consensus sequence with increased thermostability
  MISC_FEATURE (2)..(2): Xaa is Phe or Tyr
  MISC_FEATURE (3)..(3): Xaa is Lys or Arg
  MISC_FEATURE (4)..(4): Xaa is Leu or Thr
  MISC_FEATURE (5)..(5): Xaa is Asn or Met
  MISC_FEATURE (6)..(6): Xaa is Lys, Asn, Thr or Asp
  MISC_FEATURE (7)..(7): Xaa is Asn, Thr or Lys
  MISC_FEATURE (8)..(8): Xaa is lle or Tyr
  MISC_FEATURE (9)..(9): Xaa is Lys or Glu
  MISC_FEATURE (10)..(10): Xaa is Val or Ile
  MISC_FEATURE (11)..(11): Xaa is Leu or Lys
  MISC_FEATURE (12)..(12): Xaa is Thr or Ser
  MISC_FEATURE (14)..(14): Xaa is Ser, Asn, Asp or Arg
  MISC_FEATURE (16)..(16): Xaa is Phe or Tyr
  MISC_FEATURE (17)..(17): Xaa is Lys, Ser, Val or Glu
  MISC_FEATURE (18)..(18): Xaa is Ser, Asn, or Lys
  MISC_FEATURE (20)..(20): Xaa is Ser, Asn or Asp
  MISC_FEATURE (22)..(22): Xaa is Ile or Val
  MISC_FEATURE (23)..(23): Xaa is Gln or Asn
  MISC_FEATURE (24)..(24): Xaa is Lys or Arg
  MISC_FEATURE (25)..(25): Xaa is Val or Ile
  MISC_FEATURE (26)..(26): Xaa is Tyr, Lys, Ser or Arg
  MISC_FEATURE (27)..(27): Xaa is Lys, Arg or His
  MISC_FEATURE (28)..(28): Xaa is Pro, Asp or Ser
  MISC_FEATURE (29)..(29): Xaa is Phe, Leu, Lys or Met
  MISC_FEATURE (31)..(31): Xaa is His, Gln, Arg or Ser
  MISC_FEATURE (32)..(32): Xaa is His or Trp
  MISC_FEATURE (33)..(33): Xaa is Ile, Phe or Leu
  MISC_FEATURE (36)..(36): Xaa is Asp, Glu or Ser
  MISC_FEATURE (37)..(37): Xaa is Asp or Ser
  MISC_FEATURE (38)..(38): Xaa is Gly or Lys
  MISC_FEATURE (39)..(39): Xaa is Ser, Thr or Ile
  MISC_FEATURE (41)..(41): Xaa is Ile or Leu
  MISC_FEATURE (42)..(42): Xaa is Lyr or Arg
  MISC_FEATURE (43)..(43): Xaa is Cys or Thr
  MISC_FEATURE (45)..(45): Xaa is Asp, Leu or Ile
  MISC_FEATURE (46)..(46): Xaa is Asn or Asp
  MISC_FEATURE (48)..(48): Xaa is Ser, Pro or Arg
  MISC_FEATURE (49)..(49): Xaa is Phe or Leu
  MISC_FEATURE (50)..(50): Xaa is Gly or Tyr
  MISC_FEATURE (51)..(51): Xaa is Lys, Glu or Gly
  MISC_FEATURE (52)..(52): Xaa is Asp or Glu
  MISC_FEATURE (53)..(53): Xaa is Lys, Glu, Gln or Ile
  misc_feature (54)..(54): Xaa can be any naturally occurring amino acid
  MISC_FEATURE (55)..(55): Xaa is Lys or Leu
  MISC_FEATURE (56)..(56): Xaa is Ala or Ser
  MISC_FEATURE (57)..(57): Xaa is Ser, Arg or Tyr
  MISC_FEATURE (58)..(58): Xaa is Thr, Asp, Ser, Glu, Met or Asn
  MISC_FEATURE (59)..(59): Xaa is Ile, Val or Leu
  MISC_FEATURE (60)..(60): Xaa is Lys, Trp or Arg
  MISC_FEATURE (61)..(61): Xaa is Val, Arg or Ile
  MISC_FEATURE (62)..(62): Xaa is Gly or Asp
  MISC_FEATURE (63)..(63): Xaa is Asp or Ser
  MISC_FEATURE (64)..(64): Xaa is Tyr, Ile, Asp, Phe, Leu or Lys
  MISC_FEATURE (65)..(65): Xaa is Leu or Val
  MISC_FEATURE (66)..(66): Xaa is Gln, Asn or Leu
  MISC_FEATURE (67)..(67): Xaa is Gly or Ser
  MISC_FEATURE (69)..(69): Xaa is Lys or Asn
  MISC_FEATURE (70)..(70): Xaa is Val or Ile
  MISC_FEATURE (71)..(71): Xaa is Leu, Val or Thr
  MISC_FEATURE (73)..(73): Xaa is Asn or Ala
  MISC_FEATURE (75)..(75): Xaa is Ile, Leu or Asp
  MISC_FEATURE (76)..(76): Xaa is Val or Ile
  MISC_FEATURE (77)..(77): Xaa is Glu or Asn
  MISC_FEATURE (79)..(79): Xaa is Gly, Asn, Lys, Asp or Pro
  MISC_FEATURE (80)..(80): Xaa is Ile or Val
  MISC_FEATURE (81)..(81): Xaa is Tyr, Phe, Asp or Thr
  MISC_FEATURE (85)..(85): Xaa is Tyr, Phe, Asp or Thr
  MISC_FEATURE (86)..(86): Xaa is Leu or Ile
  MISC_FEATURE (87)..(87): Xaa is Asn or Asp
  MISC_FEATURE (88)..(88): Xaa is Val or Ser
  MISC_FEATURE (89)..(89): Xaa is Gly or Glu
  MISC_FEATURE (90)..(90): Xaa is Glu, Lys or Gly
  MISC_FEATURE (91)..(91): Xaa is Asp, Glu, Gly or Asn
  MISC_FEATURE (92)..(92): Xaa is Asn, Ser or His
  MISC_FEATURE (93)..(93): Xaa is Leu or Asn
  MISC_FEATURE (95)..(95): Xaa is Tyr or Ile
  MISC_FEATURE (96)..(96): Xaa is Thr or Ser
  MISC_FEATURE (98)..(98): Xaa is Gly, Asp or Asn
  MISC_FEATURE (99)..(99): Xaa is Ile, Val or Leu
  MISC_FEATURE (100)..(100): Xaa is Val or Ile
MXXXXXXXXXXXPXGXXXFXGXXXXXXXXYXXXIFXXXXEXXXSXXHXIXXXXXXXXXXXXXXXXXXKXXXYXEXXXEXXXLYDXXXXXXXXXYXXNXXXSHN

SEQ ID NO: 125 - protein, 32 aa, Artificial Sequence
  CAT N variant
FECLSGDTMIEILDDDGIIQKISMEDLYQRLA

SEQ ID NO: 126 - protein, 32 aa, Artificial Sequence
  CAT N variant
  MISC_FEATURE (3)..(3): Xaa any amino acid other than Cys, Ser or Thr
FEXLSGDTMIEILDDDGIIQKISMEDLYQRLA

SEQ ID NO: 127 - protein, 32 aa, Artificial Sequence
  CAT N variant
FEALSGDTMIEILDDDGIIQKISMEDLYQRLA

SEQ ID NO: 128 - protein, 107 aa, Artificial Sequence
  cat C variant
  misc_feature (106)..(107): Xaa can be any naturally occurring amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCXX

SEQ ID NO: 129 - protein, 107 aa, Artificial Sequence
  cat C variant
  Xaa (106)..(106): Xaa is Ala, Gly, Arg or Phe
  Xaa (107)..(107): Xaa is any amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCXZ

SEQ ID NO: 130 - protein, 107 aa, Artificial Sequence; identical to SEQ ID NO: 128
  cat C variant
  Xaa (106)..(106): Xaa is any amino acid
  Xaa (107)..(107): Xaa is Gly, Glu, Ala or Arg

SEQ ID NO: 131 - protein, 107 aa, Artificial Sequence; identical to SEQ ID NO: 128
  cat C variant
  Xaa (106)..(106): Xaa is Ala, Gly, Arg or Phe
  Xaa (107)..(107): Xaa is Gly, Glu, Ala or Arg

SEQ ID NO: 132 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEF

SEQ ID NO: 133 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCAF

SEQ ID NO: 134 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCGF

SEQ ID NO: 135 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCRF

SEQ ID NO: 136 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCFF

SEQ ID NO: 137 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEG

SEQ ID NO: 138 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEE

SEQ ID NO: 139 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCEA

SEQ ID NO: 140 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHNCER

SEQ ID NO: 141 - protein, 107 aa, Artificial Sequence
  cat C variant
  Xaa (104)..(104): Xaa is any amino acid other than Ans or Gln
  misc_feature (106)..(107): Xaa can be any naturally occurring amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCXX

SEQ ID NO: 142 - protein, 107 aa, Artificial Sequence; identical to SEQ ID NO: 141
  cat C variant
  Xaa (104)..(104): Xaa is any amino acid other than Ans or Gln
  Xaa (106)..(106): Xaa is Ala, Gly, Arg or Phe
  misc_feature (107)..(107): Xaa can be any naturally occurring amino acid

SEQ ID NO: 143 - protein, 107 aa, Artificial Sequence; identical to SEQ ID NO: 141
  cat C variant
  Xaa (104)..(104): Xaa is any amino acid other than Ans or Gln
  misc_feature (106)..(106): Xaa can be any naturally occurring amino acid
  Xaa (107)..(107): Xaa is Gly, Glu, Ala or Arg

SEQ ID NO: 144 - protein, 107 aa, Artificial Sequence; identical to SEQ ID NO: 141
  cat C variant
  Xaa (104)..(104): Xaa is any amino acid ither than Ans or Gln
  Xaa (106)..(106): Xaa is Ala, Gly, Arg or Phe
  Xaa (107)..(107): Xaa is Gly, Glu, Ala or Arg

SEQ ID NO: 145 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEF

SEQ ID NO: 146 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCAF

SEQ ID NO: 147 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCGF

SEQ ID NO: 148 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCRF

SEQ ID NO: 149 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCFF

SEQ ID NO: 150 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEG

SEQ ID NO: 151 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEE

SEQ ID NO: 152 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCEA

SEQ ID NO: 153 - protein, 107 aa, Artificial Sequence
  cat C variant
  Xaa (104)..(104): Xaa is any amino acid other than Ans or Gln
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHXCER

SEQ ID NO: 154 - protein, 107 aa, Artificial Sequence
  cat C variant
  misc_feature (106)..(107): Xaa can be any naturally occurring amino acid
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACXX

SEQ ID NO: 155 - protein, 107 aa, Artificial Sequence; identical to SEQ ID NO: 154
  cat C variant
  Xaa (106)..(106): Xaa is Ala, Gly, Arg or Phe
  misc_feature (107)..(107): Xaa can be any naturally occurring amino acid

SEQ ID NO: 156 - protein, 107 aa, Artificial Sequence; identical to SEQ ID NO: 154
  cat C variant
  misc_feature (106)..(106): Xaa can be any naturally occurring amino acid
  Xaa (107)..(107): Xaa is Gly, Glu, Ala or Arg

SEQ ID NO: 157 - protein, 108 aa, Artificial Sequence; identical to SEQ ID NO: 36
  cat C variant
  Xaa (106)..(106): Xaa is Ala, Gly, Arg or Phe
  Xaa (107)..(107): Xaa is Gly, Glu, Ala or Arg

SEQ ID NO: 158 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEF

SEQ ID NO: 159 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACAF

SEQ ID NO: 160 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACGF

SEQ ID NO: 161 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACRF

SEQ ID NO: 162 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACFF

SEQ ID NO: 163 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEG

SEQ ID NO: 164 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEE

SEQ ID NO: 165 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACEA

SEQ ID NO: 166 - protein, 107 aa, Artificial Sequence
  cat C variant
MFKLNTKNIKVLTPSGFKSFSGIQKVYKPFYHHIIFDDGSEIKCSDNHSFGKDKIKASTIKVGDYLQGKKVLYNEIVEEGIYLYDLLNVGEDNLYYTNGIVSHACER

SEQ ID NO: 167 - protein, 34 aa, Artificial Sequence
  Fl-Cat N
GEFEALSGDTMIEILDDDGIIQKISMEDLYQRLA

SEQ ID NO: 168 - protein, 31 aa, Artificial Sequence
  CAT N variant
ECLSGDTMIEILDDDGIIQKISMEDLYQRLA

SEQ ID NO: 169 - protein, 31 aa, Artificial Sequence
  CAT N variant
  misc_feature (2)..(2): Xaa any amino acid other than Cys, Ser or Thr
EXLSGDTMIEILDDDGIIQKISMEDLYQRLA

SEQ ID NO: 170 - protein, 31 aa, Artificial Sequence
  CAT N variant
EALSGDTMIEILDDDGIIQKISMEDLYQRLA